(19) United States
(12) Patent Application Publication
(10) Pub. No.: US 2011/0020279 A1
(43) Pub. Date: Jan. 27, 2011

(54) RABIES CURE

(76) Inventor: Totada R. Shantha, McDonough, GA (US)

Correspondence Address: PATWRITE LLC, 408 W. MAIN ST., MARSHALLTOWN, IA, 50158-5759, US

(21) Appl. No.: 12/893,827

(22) Filed: Sep. 29, 2010

(51) Int. Cl. A61K 38/21; A61K 38/28; A61K 39/395; A61K 38/48; A61K 33/04; A61K 39/205; A61K 31/122; A61P 31/14; A61P 25/00; A61P 37/04; A61P 31/12; A61P 33/06; A61P 25/18; A61P 23/00; A61P 25/04; A61P 3/00; A61P 3/02; A61P 25/08; A61P 25/20

(52) U.S. Cl. 424/85.4; 514/5.9; 514/6.5; 424/142.1; 424/145.1; 424/178.1; 424/94.65; 424/708; 424/224.1; 424/94.1

(57) ABSTRACT

This invention is for a method of treatment of rabies once the patient develops signs and symptoms of rabies with the intent to save the patients from death and disability using insulin combined with various anti rabies viral therapeutic, pharmaceutical, biochemical, and biological agents or compounds with added supportive therapies administered through OM, SAS, IVB, IV, and IA routes. An embodiment provides devices for intranasal delivery of therapeutic agents to olfactory mucosal area. Another embodiment uses the technology to deliver the therapeutic, pharmaceutical, biochemical, and biological agents or compounds to the subarachnoid space and ventricular system by using continuous catheters and Ommaya reservoir at the same time. The present method incorporates breaking the blood brain barrier to allow the entry of the anti rabies therapeutic agents into the neuropile. Additionally, an embodiment incorporates cooling of the brain and inducing hibernation to preserve the brain from damage due to rabies.

### FIELD OF THE INVENTION

The present invention relates to methods for curing the rabies in humans and animals that develop the full blown disease. It describes various routes of spread of rabies Virus (RV) from periphery to the central nervous system (CNS) and probable reasons for prolonged incubation period in some infected subjects. The present invention describes the routes of spread of antirabies neutralizing antibodies (ANA) to the blood, CSF, and CNS, produced by the rabies patient's immune system and/or administered parentarily.

### BACKGROUND OF THE INVENTION

Rabies is a lethal disease caused by neurotropic viruses that are endemic in nature. The rabies virus belongs to the Lyssavirus genus which includes similar viruses. Lyssaviruses have helical symmetry with a length of about 180 nm and a cross-sectional diameter of about 75 nm. These viruses are enveloped and have a single stranded RNA genome with negative-sense. The genetic information is packaged as a ribonucleoprotein complex which RNA is tightly bound by the viral nucleoprotein.

The advances in scientific medicine makes rabies controllable by prophylactic pre or post exposure prevention vaccine (PEP), which has no effect in individuals, who develop the disease because they didn't get the vaccination on time. Exposure to a potentially rabid animal is recognized, prompt cleaning of wound, administration of virus-neutralizing antibodies, together with active immunization, can prevent development of the disease. Unlike, other immunizations, the rabies vaccine are administered after exposure to the virus. This unusual technique is successful because the rabies virus takes a comparatively long time to induce disease which is a minimum of 10 days, and in rare cases, months and years. However, once the nonspecific clinical symptoms of rabies appear; the conventional post exposure vaccination is unsuccessful. The well publicized successful treatment in one teenager that was used in Wisconsin to treat bat rabies failed in subsequent cases (Willoughby R E Jr, Tieves K S, Hoffman G M, et al. Survival after treatment of rabies with induction of coma. N Engl J Med 2005; 352: 2508-14). This indicates that there isn't a cure for this dreaded fatal disease.

The present invention relates in general to the fields of route taken by the rabies virus (RV), mechanism involved that delay the development of the disease for months and occasionally years, and the treatment of the disease. Worldwide, there are about 55 000 human deaths (35000 in Asia and 30,000 in Africa) each year due to rabies (World Health Organization, 2005). Rabies is 100% preventable and is 100% fatal. The present invention relates to novel methods of treatments of rabies. It uses new methods and routes of administration of therapies using special invented device and therapeutic agents (therapeutic, pharmaceutical, biochemical, nutraceuticals, biological agents or compounds, and drugs). The new therapy delivery pathways and maintenance therapy allows enough time for the nonspecific (innate) and specific (adaptive) immune system to clear the virus from the brain and the rest of the body which may prevent impending death. This present invention relates to intranasal olfactory mucosal (OM), sub arachnoid intrathecally into the cerebrospinal fluid (SAS-CSF), cerebral intra ventricular (IVB), delivery of therapeutic agents through special catheters and devices. Our invention integrates intravenous-intra arterial (IV, IA) and administration of therapeutic agents after breaking the blood brain barrier (BBB). The therapy induces brain hypothermia which is a state of suspended animation of the rabies infected brain for time to allow the antirabies neutralizing antibodies (ANA) to develop in the brain to clear the rabies virus from the brain and to preserve the brain at the same time.

There are excellent monographs published on the rabies History by Dr. Baer (George M. Baer: The Natural History of Rabies, 2nd Edition 1991, CRC press). The latest 660 page book on all aspect of rabies starting from the history to treatment of this incurable deadly disease by Jackson and Wunner (Alan P. Jackson and William H. Wunner, 2007 published by Academic press) is one of the most important authoritative informative contributions on the Rabies. It incorporates history, virology, immunology, human rabies, bat rabies, animal rabies and their control, vaccination, and the latest in the treatment of full blown human rabies. Selected information from this revealing book by Jackson and Wunner are incorporated herein.

Since the distant past, rabies has been one of the most feared diseases. Human rabies remains an important public health problem in many developing countries where dog rabies is endemic. Rabies is a fatal central nervous system (CNS) disease without a cure even in the 21^{st} century. In mammals, rabies is caused by a RNA rabies virus (RV) and a neurotropic lyssavirus from the family of the rhabdoviridae. Generally, RV is transmitted by scratches or bites of rabid land animals or flying bats which results in the dissemination of virions into skin and muscle tissue. After initial infection of cells at the infection site, RV enters axon terminals (FIGS. 1-10), and migrates by retrograde axonal transport into the CNS (Bulenga G, Heaney T (1978) Post-exposure local treatment of mice infected with rabies with two axonal flow inhibitors, colchicine and vinblastine. J Gen Virol 39: 381-385. Kelly R M, Strick P L (2000) Rabies as a transneuronal tracer of circuits in the central nervous system. J Neurosci Methods 103: 63-71) where it causes a fatal encephalopathy.

The incubation period can vary from days to years; though, it is not known where the virus is located during this lengthy incubation period. (Smith, I S., Fishbein, D. B. Rupprecht, C. E. and Clark, K. (1991). Unexplained, rabies in three immigrants in the United States: a virologic investigation. New England journal of Medicine 324, 205-21. McColl K A, Gould A R, Selleck P W, Hooper P T, Westbury H A, et al. (1993) Polymerase chain reaction and other laboratory techniques in the diagnosis of long incubation rabies in Australia. Aust Vet J 70: 84-89). It is likely that the virus introduced into damaged muscle or skin tissue after a bite is disseminated into the blood and transported via blood to the CNS. Such an event could play a role in virus transmission by silver-haired bats where only few virus particles are minimally invasively introduced into small patches of skin which have few intra sub epidermal nerve fibers (FIG. 1). Therefore are not favorable for neuronal uptake (Gibbons R V (2002) cryptogenic rabies, bats, and the question of aerosol transmission. Ann Emerg Med 39: 528-536. Hemachudha T, Laothamatas j, Rupprecht C E (2002) Human rabies: a disease of complex neuropathogenetic mechanisms and diagnostic challenges. Lancet Neurol 1: 101-109).

In contrast to natural RV infections, experimental RV infections are commonly done by intramuscular (foot pad), intranasal or intra cerebral inoculation. The injection into muscle probably imitates best natural infections which it causes less local damage of skin, muscle tissue and microvasculature than an animal bite. Incidental hematogenous spread due to injury of vessels is less likely than in natural neurological transmissions. The latest studies do show that the pathogenetic relevance of hematogenous RV spread due to viremia (Mirjam A. R. Preuss, Marie-Luise Faber, Tan G S, Bette M, Dietzschold B, et al. (2009) Intravenous Inoculation of a Bat-Associated Rabies Virus Causes Lethal Encephalopathy in Mice through Invasion of the Brain via Neurosecretory Hypothalamic Fibers. PLoS Pathogens 5(6)).

The infectious cycle of the rabies virus is perpetuated through animal bites and the deposition of rabies virus-laden saliva into subcutaneous tissues and muscle (FIGS. 1, 2, and 3). It is rare to be infected by the air droplets (bat caves). Incubation period and transmission of rabies virus is described in details by Alan C. Jackson in chapter 7 under “Human disease” of the book by Jackson and Wunner (Elsevier Academic press, 2007, pp 309-311) which are incorporated herein.

Globally, dogs are the most common and important rabies vector. The bats in the USA, South America and Canada are the vector. There are reservoirs in various earthly terrestrial animals. Handling and skinning of infected carcasses and possibly consumption of raw infected meat have resulted in transmission of rabies virus (Tariq, W. U. Z., Shafi, M. S., Jamal, S. and Ahmad, M. (1991). Rabies in man handling infected calf (Letter). Lanet 337, 1224. Kureishi, A., Xu, L. Z., Wu, H. and Stiver, H. G. (1992). Rabies in China: recommendations for control. Bulletin of the World Health Organization 70, 443-450. Wallerstein, C. (1999). Rabies cases increase in the Philippines. British Medical Journal 318, 1306).

Rarely, inhalation of aerosolized (saliva and excreta droplets) rabies virus (FIGS. 7-10) in caves containing millions of bats has been reported to cause rabies (Constantine, D. G. (1962). Rabies transmission by non bite route. Public Health Reports 77, 287-289. Constantine, D O, Emmons, R. W, and Woodie, J. D. (1972). Rabies virus in nasal mucosa of naturally infected bats. Science 175, 1255-1256. Constantine, D. G. (1988). Transmission of pathogenic organisms by vampire bats. In: Natural History of Vampire Bats (A. M. Greenhall and U. Schmidt, eds). pp. 167-189. Boca Raton: CRC Press).

A transmission in laboratories to the laboratory handlers in the infected animals has resulted in human rabies (Winkler, W. G., Fashinell, T. R., Leffingwell. L., Howard. P. and Conomy. J. P. (1973). Airborne rabies transmission in a laboratory worker. Journal of the American Medical Association 226, 1219-1221. Tillotson, J. R., Axelrod, D. and Lyman, D. O. (1977a). Follow-up on rabies—New York. Morbidity and Mortality Weekly Report 26, 249-250.)

Handling and skinning of infected carcasses and consumption of raw infected meat have resulted in transmission of rabies virus (Tariq. W. U. Z., Shall, M S., Jamal, S. and Ahmad, M. (1991). Rabies in man handling infected case (Letter). Lancet 337. 1224. Kureishi, A., Xu. L. Z., Wu, H. and Stiver. H. G. (1992). Rabies in China: recommendations for control. Bulletin of the World Health Organization 70. 443-450. Wallerstein, C. (1999). Rabies cases increase in the Philippines. British Medical journal 318, 1306.)

There aren't many reports of rabies cases transmitted from person to person which may be seen in transplant cases. There are more with corneal transplant than other organs. Eight cases of rabies have resulted from transplantation (human-to-human) of rabies virus-infected corneas. In 2004, organ transplantations in Texas were associated with transmission of rabies virus and the development of fatal rabies in four recipients (Srinivasan. A. Burton. E. C. Kuehnert, M. J. et al. (2005). Transmission of rabies virus from an organ donor to four transplant recipients. New England journal of Medicine 352. 1103-1111). The donor in this case was treated for gastrointestinal symptoms, with periods of confusion and agitation and with trunk movements. His lungs, kidneys, liver and iliac vessels were harvested and transplanted. All the recipients died and three of the four recipients had antibodies on postoperative days 35 and 36.

It is obvious that the use of Immune suppression to prevent organ rejection results in a favorable environment for viral replication and for spread. History revealed that the donor had been bitten by a bat with antigenic typing. Indication is that the rabies virus variant was associated with Brazilian (Mexican) free-tailed bats. Transmission occurred from a donor to organ transplant recipients in Germany that resulted in three fatal cases in 2005 (Johnson, N., Brookcs, and S. M. Fooks. A R. and Ross. R. S. (2005). Review of human rabies cases in the UK and in Germany. Veterinary Record 157, 715). In case from Ethiopia, a 41-year-old female died of rabies 33 days after her 5-year-old son died of rabies; he had bitten his mother on her little finger (Fekadu, M., Endeshaw, T., Alemu, W. Bogale. Y., Teshager. T. and Olson. J. G. (1996). Possible human-to-human transmission of rabies in Ethiopia. Ethiopian Medical Journal 34. 123-127).

Sexual transmission of rabies virus isn't documented. Trans-placental transmission of rabies virus exists in a single report from Turkey (Sipahioglu. U. and Alpaut, S. (1985). Trans-placental rabies in a human. Turkishi. Mikrobiyoloj Bulteni 19, 95-99).

Rabies virus being a neurotropic virus that doesn't get into the nerve structure which is connected to CNS, the disease doesn't strike. There has to be contact with peripheral nerves to spread to the CNS. The important role of the peripheral nerve structure plays is described. This was showed in simple experimental studies at CDC of Atlanta by Baer, Shantha and Bourne.

They showed that

1. Sciatic Nerve section before rabies virus injection prevents its spread.

2. Cutting nerve 10 hours after injection does not prevent the spread, but if cut before 10 hours, it does prevent the spread.

3. Crushing the nerve, resulting in Wallerian degeneration doesn't prevent the viral spread indicating that the intact axon isn't essential for RV spread.

4. Injection of rabies antiserum around and underneath the Perineural epithelium didn't prevent the spread of the virus.

5. Removal of the Perineural epithelium slows down the viral spread but doesn't prevent spread. These findings points that the Schwann cells, the endoneurium, or associated tissue spaces without the axonal components can transmit the RV to the CNS.

6. Studies showed that there is hardly any inflammation of the sciatic nerve, where RV travels along the nerve indicating, that the immune system is unable to recognize the virus inside axons and the nerve structures (Baer, G. M., Shanthaveerappa, T. R. and Bourne, G H. (1965). Studies on the pathogenesis of fixed rabies virus in rats. Bulletin of the World Health Organization 33, 783-794. Baer, G. M., Shantha, T. R. and Bourne, G. H. (1968). The pathogenesis of Street rabies virus in rats. Bulletin of the World Health Organization 38, 119-125. Shantha T R and Bourne G H: The “Perineural Epithelium”: A new concept. Its role in the integrity of the peripheral nervous system. In Structure and Function of Nervous Tissues. I. (G H Bourne, Ed.). Academic Press, New York. 1969).

Blood vessels are a structural presence of the blood brain barrier (BBB-FIGS. 21, 22). The blood vessels may not play a role in the spread of RV. The matter of histology may delay or prevent the rapid spread of the virus to the CNS through the hematogenous route. Studies were presented about the transportation of the rabies to CNS from the olfactory mucosa, taste buds, and intestinal wall (Nakajima Y, Shantha T R and Bourne G H: Histological and Histochemical studies on the subformical organ of the squirrel monkey. Histochemie 13:331-345 (1968). Iijima K, Shantha T R and Bourne G H: Histochemical studies on the distribution of some enzymes of the glycolytic pathways in the olfactory bulb of the squirrel monkey (Saimiri sciureus). Histochemie 10:224-229 (1967). Shantha T R. Nasal and Oral route of transmission of Rabies virus and possible treatment to cure the rabies. Hanoi: RIACON—Rabies in Asia conference in Hanoi on Sep. 10, 2009. T. R. Shantha. On route of transmission of rabies virus and treatment to cure the disease on October, 19th 2009 RITA XX: Conference held in Ottawa city, Canada).

The incubation period for human rabies is often 20-90 days after exposure. The disease can develop even earlier (Anderson, L. J., Nicholson, K. G., Tauxe, R. V. and Winkler, W. G. (1984) or years later. Human rabies in the United States, 1960 to 1979: epidemiology, diagnosis, and prevention. Annals of Internal Medicine 100, 728-735). Three immigrants from Laos, the Philippines and Mexico developed rabies in the USA of at least 11 months, 4, and 6 years, which were based on the time of their immigration (Smith, J. S., Fishbein, D. B. Rupprecht, C. E. and Clark, K. (1991). Rabies was unexplained in three immigrants in the United States: a virologic investigation. New England journal of Medicine 324, 205-211). A case of rabies in a 10-year-old Vietnamese girl in Australia in 1990 was likely acquired at least 5 years earlier (Bek, M. D., Smith, W. T., Levy, M. H., Sullivan. E. and Rubin, G. L. (1992). Rabies case in New South Wales. 1990: public health aspects. Medical Journal of Australia 156, 596-600. McColl. K. A., Gould, A. R., Selleck, P. W., Hooper, P. T., Westbury, H. A. and Smith, J. S. (1993). Polymerase chain reaction and other laboratory techniques in the diagnosis of long incubation rabies in Australia. Australian Veterinary Journal 70, 84-89). These reports demonstrate that the incubation period (from exposure to onset of disease) in rabies is variable compared to any other infectious diseases.

The biologic basis for such variability in the incubation period isn't known. We explored the histological basis and the reasons RV takes almost six years in rare cases to come in contact with the CNS resulting in the cause of the fatal encephalitis. It is likely that there are many factors involved in such a protracted incubation period, which is discussed below under explanation of FIG. 2.

If the virus is picked up by the capillaries and small blood vessels of the nerve fasciculi, they can reach the axons through the Virchow-Robin perivascular space of the BV then infect the axons (Shantha T R: Peri-vascular Virchow—Robin space in the peripheral nerves and its role in spread of local anesthetics, ASRA Congress at Tampa, Regional Anesthesia 17 (March-April, 1992). If they get inside the nerve fasciculi (PNS—nerve bundle), they have to get access inside the axon to spread retrograde to the CNS. The Myelin sheath around the axons acts as a formidable barrier to let the RV enter the axoplasm. The rabies virus needs to find or come in contact with the node of Ranvier (which has no myelin sheath) of the axon which will act as door for the entry of the virus inside the axon. The small nerve fibers which are surrounded by the Schwann cells without myelin like olfactory nerves (FIG. 8) can be infected easily without hindrance to the entry of RV inside the axon.

The most elusive route debated is inhalation route and oral routes in transfer of RV. Our studies have shown that the olfactory mucosa and its connection to the CNS. Their role in transmission of microbes including RV (Shantha T. R. and Yasuo Nakajima: Histological and Histochemical Studies on the Rhesus Monkey (Macaca Mulatta) Olfactory Mucosa. Z. Zellforsch. 103, 291-319 (1970). Nakajima Y, Shantha T R and Bourne G H: Histological and Histochemical studies on the subformical organ of the squirrel monkey. Histochemie 13:331-345 (1968)). We postulated that the olfactory mucosa can conduct microbes including RV. The olfactory mucosa can transport therapeutic, pharmaceutical, biochemical, and biological agents or compounds directly to and from the brain, the direct connection as shown in the diagrams (FIGS. 7-10). The rabies virus gets attached to the amorphous mucus material on the lining of the olfactory mucosa and gets entangled and endocytosed (FIG. 7).

We found that the olfactory mucosa is constantly loosing receptor cells at the rate of 10,000 every year (30 of them every day). There are spaces (inter-receptor spaces) left by these dying, dead, and regenerating neuroreceptor cells (FIG. 7) without any tight junction at the surface between the supporting cells and receptor cells. The olfactory mucosa becomes a sieve, which allows the substances like the therapeutic agents and infecting agents like RV to pass to the olfactory nerves, olfactory bulb, cerebrospinal fluid (CSF) in the subarachnoid space (SAS), and to the brain (CNS) (FIGS. 7-10). This fact is ignored by most researchers. These gaps with the rest of the olfactory mucosa plays a role in transmission of the RV and the delivery of the therapeutic agents to the brain to counter the infection as described in this invention.

There are two routes taken by the rabies virus and the therapeutic agents from the olfactory mucosa 1. Passing through the axons of the olfactory receptors directly and the; 2. Second route is Perineural epithelial space directly into subarachnoid space (FIGS. 7 and 8) and to the rest of the CNS which are bathed in the CSF. The RV is a neurotropic virus which it takes the route of entering inside the axons of olfactory mucosa (OM). The rabies virus takes a journey to the CNS through the olfactory nerves. The therapeutic agents administered on OM pass through the inter-epithelial spaces into sub Perineural spaces (FIGS. 7, 8, and 9) to the CNS intrathecal SAS spaces. The agents then are distributed over the subarachnoid space which surrounds the entire CNS and the spinal cord.

In similar fashion, our studies show that the olfactory-mucosa, olfactory bulbs, taste buds (FIG. 11), and the intestinal mucosa (FIG. 16) conduct the rabies virus as the nerve terminals are exposed and will act as easy conduit to RV to CNS.

Once the rabid animal bites, there are prophylactic vaccines to prevent the spread of the RV to CNS called post-exposure prophylaxis (PEP). Before 1885, all human cases of rabies were fatal, until, a vaccine was developed in 1885 by Louis Pasteur and Émile Roux. Their original vaccine was harvested from infected rabbit's spinal cord which the virus in the nerve tissue was weakened (attenuated) by allowing it to dry for five to ten days. This vaccine was first used successfully in a human on Jul. 6, 1885, on a nine-year old boy Joseph Meister (1876-1940) who had been mauled by a rabid dog.

Since 1967, this old vaccine is replaced:

1. By much safer human diploid cell rabies vaccine (HDCV).

2. Less expensive purified chicken embryo cell vaccine (PCECV) and with purified vero cell rabies vaccine.

Worldwide, there are 4 million people vaccinated each year for rabies. Each year approximately 18,000 people in the U.S. receive vaccination and immune globulin. These patients didn't developed rabies. World Rabies Day is observed on September 28^{th} each year. This is a global initiative to raise awareness about the continuing burden of rabies. The disease can be prevented and be controlled with the awareness; with immediate wound care and the administration of rabies immune globulin (RIG) plus the vaccine. The PEP is highly effective in preventing human rabies following the exposure. That is why rabies is called “100% preventable and 100% fatal”. These methods are well elaborated in CDC protocol who play a major role in rabies prevention and treatment modalities all over the world. They are followed as prophylaxis all over the world. In the absence of treatment, infection with a variety of rabies virus (RV) strains results in a lethal outcome. This can be prevented by at the immediate and quick immunization following exposure which results in the development of anti-rabies viral immunity prior to spread and extensive infection of neurons.

The role of rabies vaccine is to induce a sustained antibody and cell mediated response with the help of CD4+ T lymphocyte activation. Humoral immunity has a protective function in the course of rabies virus infection. Anti rabies virus neutralizing antibodies (ANA), under the control of T helper cells, plays a decisive role in immunoprotection. The glycoprotein (G) of RV is responsible for the induction of virus neutralizing antibodies.

The RNA genome of the virus encodes five genes whose order is highly conserved: glycoprotein (G), nucleoprotein (N), phosphoprotein (P), matrix protein (M), and the viral RNA polymerase (L) (Finke S, Conzelmann K K (2005). “Replication strategies of rabies virus”. Virus Res. 111 (2): 120-31). RV is a negative-stranded RNA virus of the rhabdovirus family. The N, P, and L proteins together with the genomic RNA form the ribonucleoprotein complex (RNP). Glycoprotein (G) is the only RV antigen capable of inducing the production of rabies virus neutralizing antibodies (ANA) which are the major immune effectors against a lethal RV infection. The RNP complex, a major RV antigen is capable of inducing CD4+ T cells that can augment the production of rabies virus neutralizing antibodies through Intrastructural antigen recognition. The RNP may play a significant role in the establishment of immunologic memory and long-lasting immunity after PEP vaccination. Our invention of using insulin with human diploid rabies will enhance these antiviral effects of PEP.

It is unlikely that RV triggers a primary adaptive immune response in the CNS when it reaches the brain which it is minimal and ineffective. This incapacity results from the absence of lymphoid structures, the lack of professional antigen presenting cells in the CNS, and the BBB which make the CNS an immunoprivileged site. After entry into the nervous system, rabies virus isn't accessible to the immune response induced by the vaccination and by the immunotherapy because the blood-brain barrier restricts the passage of neutralizing antibodies (Murphy, F. A. 1977. Rabies pathogenesis. Brief review. Arch. Virol. 54:279-297). It is here our invention plays a major role evading the BBB by ANA and other therapeutic, pharmaceutical, biochemical, and biological agents or compounds.

Pathogenic strains of RV have selected immunosubversive strategies to escape the host immune response. The host's natural capacity to fight the well-adapted virus is limited making this disease 100% fatal. Innate immunity is the first line of defense against invading pathogens. It involves the release of cytokines resulting in the early signs and the symptoms of viral encephalitis. Ultimately, this results in the activation of complement and the attraction of macrophages, neutrophils and natural killer (NK) cells into infected tissues.

This innate non specific immune response is triggered in the first hours following the entry of pathogens or vaccine antigen. It isn't pathogen specific. This is followed by adaptive specific immune response consisting of Humoral (antibody) and cell mediated (T cell) which is customized to a specific pathogen that requires several days to be set up. In case of rabies, it is too late, the rabies virus hides from the mounting attack and the patient succumbs.

The highly pathogenic rabies viruses have characteristics that avoid triggering protective immune responses (Hooper D C. The role of immune responses in the pathogenesis of rabies J Neurovirol. 2005 February; 11(1):88-92). It is important to attend to rabid animal bites or bat exposures immediately. It is important to note that the CNS has no organized lymphatic system. The CNS doesn't have circulating leukocytes. There is a paucity of major histocompatibility molecule expressions which there aren't antibodies or complement components available. As a result, the CNS is referred to as an immunoprivileged site. It is known that the CNS can mount florid inflammatory responses: example: Viral encephalitis, Astrocytes can synthesize complement components, and stimulate cytokine production. Lymphocytes, T and B cells can enter the CNS. Lymphocytes have the ability to enter the CNS under normal physiological conditions and during infections of CNS.

Constant traffic of activated lymphocytes allows the immune system to monitor the CNS for infections. The activation of the lymphocytes won't encounter their specific antigen. They will either exit or undergo apoptotic cell death within the CNS. They should encounter their antigen that will trigger an inflammatory response. The number of activated lymphocytes trafficking through the CNS at any one time is very small explaining that these cells aren't generally observed in rabies. This is the reason that the rabies can take cover and can be concealed from the immune system response.

Rabies post exposure prophylaxis (PEP) schedule in United States 2008 (CDC protocol) is posted on the internet to be adopted by all the countries. Guidelines are posted on the method of pre exposure and post exposure on the internet. The patient needs to follow these standard tested guidelines. Recommendations of the Advisory Committee on Immunization Practices are posted at “Morbidity and Mortality Weekly Report. www.cdc.gov/mmwr. Early Release. May 7, 2008; Vol. 57, Human Rabies prevention—United States, 2008, Rupprecht C E, Gibbons RV Clinical Practice. Prophylaxis against Rabies. N Engl J Med, Dec. 16, 2004, Vol. 351(25):2626-2635.

Persons in the continuous-risk category should have a serum sample tested for rabies virus neutralizing antibody every 6 months. These persons in the frequent-risk category should be tested every 2 years. An intramuscular, subcutaneous, or intradermal booster dose of vaccine should be administered, if the serum titer falls to maintain a value, at least complete neutralization at a 1:5 serum dilution by rapid fluorescent focus inhibition test.

Once, a person is exposed to the rabies virus and does not get PEP, the person develops Rabies. There isn't a cure for the person whom the person will succumb to the Rabies Virus. High neuroinvasiveness of street RVs is, at least in part, due to their ability to evade immune responses, and to conserve the structures of neurons resulting in negligible damage to the CNS.

The CNS is immune and protected which the immune system doesn't allow a full blown attack of the immune system by the invading virus. Recent, studies have shown, during lethal rabies infection, that the blood-brain barrier (BBB) doesn't allow anti-viral immune cells to enter the brain. The brain is the primary site of rabies virus replication (Roy A, Phares T W, Koprowski H, Hooper D C (2007). “Failure to open the blood-brain barrier and deliver immune effectors to central nervous system tissues leads to the lethal outcome of silver-haired bat rabies virus infection”. J. Virol. 81 (3): 1110-8. doi:10.1128/JVI.01964-I06).

This aspect contributes to the pathogenicity of the virus which artificially increasing BBB permeability promotes viral clearance (Roy A, Hooper D C (2007). “Lethal silver-haired bat rabies virus infection can be prevented by opening the blood-brain barrier”. J. Virol. 81 (15): 7993-8). Their study suggests that due to BBB of the CNS blood vessels where the invasion of central nervous system (CNS) tissues by immune cells is reduced consequently, the virus is not cleared from the CNS. Further, the circulating antirabies antibodies (ANA) don't pass through the BBB which protects the rabid rabies infection from the brain.

A novel approach has been suggested where the rabies infection would enter the BBB to treat the infection. There haven't been any attempts or success at this time. It is the intent of this invention to open the BBB and to allow the immune cells, neutralizing antirabies antibodies, and the other therapeutic agents which are the specific human antirabies monoclonal antibodies (MAB) to enter the CNS. The break in the BBB to clear the infection and possibly to cure the disease and/or minimize the damage caused by the RV.

Many of the patients (total of ten to date) who survived the rabies had high serum neutralization titer against the rabies virus which peaked at 1:640 000 at about 3 months. They had very high titers of neutralizing antibodies in the CSF. The Rabies Virus wasn't isolated from saliva or CSF and corneal impression smears in some of these cases indicating that the ANA is produced naturally (ANA) is very effective in clearing rabies virus.

Rabies virus antibodies appear in the CSF which are lower than the serum ANA. Very high titers of rabies virus antibodies in the CSF have been interpreted as evidence of rabies encephalitis in vaccinated patients (Madhusudana, S. N., Nagaraj, D., Uday, M., Ratnavalli. E. and Kumar. M. V. (2002). Partial recovery from rabies in a six-year-old girl (Letter). International journal of Infectious Diseases 6, 85-86).

The recorded treatment and history which only less than 10 patients have survived the actual rabies. Many of them are left with severe neurological disability. Most of them have received the prophylactic treatment. Only one case of rabies survived with intensive care treatment with use of Ketamine in USA with induction of coma. This case didn't receive prophylactic vaccine earlier (Willoughby, R. E. Rotar, M. M., Dhonau, H. L. et al. (2004). Recovery of a patient from clinical rabies—Wisconsin, 2004. Morbidity and Mortality Weekly Report 53, 1171-1173. Willoughby. R. E. Jr, Tieves, K. S., Hoffman, G. M. et al. (2005). Survival after treatment of rabies with induction of coma. New England journal of Medicine 352, 2508-2514).

Unfortunately, subsequent human rabies treatments using similar protocol of Willoughby et al, using Ketamine didn't cure the disease. The patients succumbed to this dreaded infection. This indicates that the Ketamine isn't the answer. There is more to be learned from these rabies virus infected cases. After, the rabies develops there was no cure at that time.

The term neuropile (neuropil) in the following description refers is an intricate, complex, net of axonal, dendritic, glial arborizations, and Microglial cells. There are 400 miles of blood vessels with BBB that forms the bulk of the central nervous system's gray matter which the nerve cell bodies lie surrounded and embedded. The white matter is mostly composed of axons and glial cells that is, generally, not considered to be a part of the neuropile. The attack to cure rabies should focus on neuropile.

### SUMMARY OF THE INVENTION

The present invention describes the rabies virus enters the motor end plate, axons, nerve fasciculi, other motor and sensory end organs.

The present invention relates to the description of routes taken by the rabies virus to reach the CNS.

The present inventive method describes the rabies virus can be transported to the CNS through the olfactory mucosa, taste buds, and intestines.

The present invention describes that some patients have protracted incubation period of months and years before developing the disease.

The present invention describes route of the antirabies neutralizing antibodies spread to reach the CSF of the spinal cord and SAS of the rest of the brain.

The present invention describes the use of insulin with various antirabies therapeutic, pharmaceutical, biochemical, and biological agents or compounds to treat the disease through OM, SAS, IVB, IV, and IA routes.

The present invention describes the use of insulin through the olfactory mucosa for augmentation—amplification effects to deliver the antirabies therapeutic agents directly to the brain and CSF.

The present invention describes the use of insulin with therapeutic, pharmaceutical, biochemical, and biological agents or compounds delivered to the SAS and CSF (intrathecal) through a catheter inserted to the SAS or to cysterna magna.

The present invention describes the use of insulin with therapeutic, pharmaceutical, biochemical, and biological agents or compounds delivered to the ventricles and the central canal of the spinal core of the brain through an Ommaya reservoir.

The present invention describes the use of insulin with ketamine, MK-801, and caspase with other therapeutic agents introduced into olfactory mucosa (OM), subarachnoid space CSF (SAS), and intra ventricular system of the brain (IVB).

The present invention describes the use of insulin with biopterin introduced to the OM, SAS, IVB, Oral and IV which is delivered to the brain as a neuroprotector and to replace the depleted biopterin in the rabies infected brain.

The present invention describes the use of insulin with intranasal OM, SAS, IV, and IVB administration of antirabies therapies, especially, the human monoclonal antibodies (HMAB) as well as anti TNF and MAB (Etanercept) to reduce the brain inflammation. The use of neurotrophic factors are IGF-1, procrit (Epotin), platelet growth factors, and other therapeutic agents are also described.

The present invention describes the use of insulin with other methods of breaking the BBB therapies and breaking the 400 miles of formidable BBB strong hold on the CNS. This will allow the therapeutic, pharmaceutical, biochemical, biological agents or compounds, and ANA into the neuropile to clear the rabies virus from the brain and to preserve the brain.

The present invention describes the use of insulin to deliver the therapeutic agents into the neuropile and to disrupt the neurotubules which are needed for dissemination of rabies virus in the CNS by the use of colchine and vinblastine.

The present invention describes the method of putting the Brain into hibernation using suffer dioxide.

The present invention describes the method of cooling the brain through the nose, and nasopharynx to protect the rabies virus infected brain. This will inhibit the rabies virus multiplication and will prevent the spread of the virus that are responsible for destruction and the death of the afflicted.

### BRIEF DESCRIPTION OF THE DRAWINGS

FIG. 1 is the drawing of the sensory end organs 1 in the skin which come in contact with the rabies virus.

FIG. 2 is the drawing of the motor end plate (MEP) 2 and the axon which attaches to the muscle fiber that transmits rabies virus.

FIG. 3 is the drawing of the muscle spindle 3 showing that it is tightly enclosed by multiple layers of Perineural epithelium.

FIG. 4 is the drawing of the nerve fasciculi 4 showing Perineural epithelium, and blood vessels with formation of Virchow Robin space.

FIG. 5 histological transverse sections of the nerve fasciculi 5 showing the structure of the peripheral nerve fasciculi surrounded by Perineural epithelium.

FIG. 6 is the drawing of the longitudinal section of the eyeball 200, including the optic nerve dura mater, leptomeninges and sclera, choroid, ciliary body, iris and cornea.

FIG. 7 is the drawing of the section of the olfactory mucosa.

FIG. 8 is the modified electron micrograph of the olfactory nerves

FIG. 9 is the drawing 9 of the longitudinal section of the olfactory bulb showing the route taken by the rabies virus.

FIG. 10 is the drawing of the section of the olfactory bulb 10 showing the route taken by the rabies virus after inhalation from the olfactory mucosa.

FIG. 11 is the drawing of the section of the taste buds 11 showing entry of RV to the axons of the taste bud nerve supply.

FIG. 12 is the drawing 12 showing the CSF circulation with catheter in SAS to deliver therapeutic agents.

FIG. 13 is the drawing showing Ommaya reservoir in place to inject antirabies therapeutic agents in to the ventricles.

FIG. 14 is the drawing of the sites of action 14 of therapeutic agents introduced into the Ventricles and SAS.

FIG. 15 is the drawing of the location of the circumventricular organs 15 which may play a role in hematogenous spread of the rabies virus and therapeutic agents.

FIG. 16 is a section of a small intestine showing the intestinal villi with lamina propia containing plasma cells producing antibodies.

FIG. 17 is diagram of the skin 17 being covered Langerhans immune defense cells located in the epidermis.

FIG. 18 is the diagrammatic presentation of the inventive device to be used which will deliver the therapeutic agents to the nasal cavity close to the olfactory mucosal and trigeminal nerves.

FIG. 19 is the diagram of the medial wall of the nasal cavity 19 and various nerve structures that the RV and therapeutic agents' come in contact.

FIG. 20 is the diagram of the lateral wall of the nasal cavity 20 showing various nerve structures that the RV and therapeutic agents' come in contact and transmitted to the CNS retrograde.

FIG. 21 is diagram of the brain capillary cross section 21 showing the components of the blood brain barrier (BBB).

FIG. 22 similar to the FIG. 21 showing the breaking of the BBB resulting in leaking (arrows) of the various cellular, liquid, rabies antibodies, and therapeutic agents.

### DETAILED DESCRIPTION OF THE INVENTION

In the above and following detailed description of the invention, reference is made to the drawings, microphotographs, and tables in which reference numerals refer to like elements. These are intended to show by the way of illustration the specific embodiments which the invention described using insulin, and/or IGF-1 with other known anti rabies therapeutic, pharmaceutical, biochemical, and biological agents or compounds enumerated here may be prescribed and practiced. It is understood that other embodiments may be utilized that structural changes may be made without departing from the scope and spirit of the invention described herein. The following figures describe the method of transfer of rabies virus from periphery to the CNS and vice-versa. This helps to understand our invention which the antirabies therapeutic agents need to be delivered directly into the neuropile through OM, SAS, IVB, intravenous (IV) or intra arterial routes (IA), after, breaking the blood brain barrier (BBB).

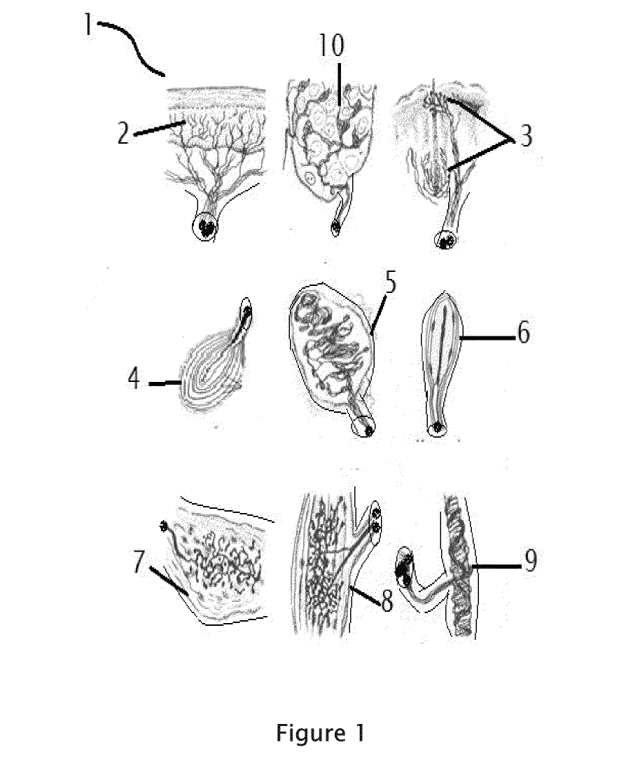

FIG. 1 is the drawing of the sensory end organs 1 in the skin. The saliva contaminated with the rabies virus is deposited in the skin by an animal or bat bite. It has easy accesses to naked free nerve ending of axons 2 of the skin and tactile nerve fibers around the hair follicles 3. The end organs Pacinian corpuscles 4, Meissner's corpuscle 5, Krause's corpuscle 6, Ruffini's end-organ 7, are covered Perineural epithelium which the RV has to overcome to enter the axons of these end organs. If the nerve trunks supplying, the end organ 4 to 10 are injured, then, the rabies virus can get into the axon which it spreads retrograde to CNS. Due to Perineural epithelial covering of these end organs, the rabies doesn't have easy access to these end organs. The tendon apparatus 8, and the Muscle spindle 9 in the muscles and the tendons are covered by Perineural epithelial covering making more difficulty for RV to enter (Modified from Shantha and Bourne, Science 154:1464-1467 (1966), The American journal Of Anatomy. Vol. 112, No. 1, January 1963, Pages 97-107, and Ham Histology).

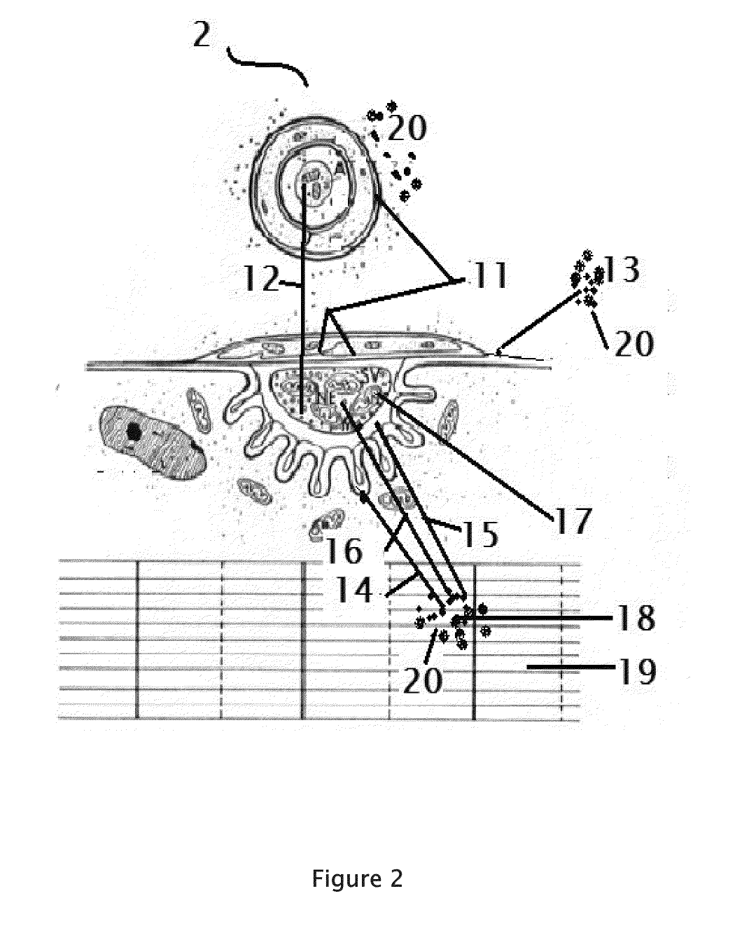

FIG. 2 is the drawing of the motor end plate (MEP) 2 which the axon attaches to the muscle fiber. Note: that the single axon comes close to the muscle fiber is surrounded by Perineural epithelial cells 11. It gets attached to the muscles as motor end organ. It is covered by a teloglial cells which is the extension of the Perineural epithelium (PE) 11. It isn't a Schwann cell which it was thought. It completely covers the expanded axon at the myoneural junction trough. It acts as an impediment for RV and other extracellular contaminants to penetrate the motor end plate which they enter the axon (after Shantha and Bourne in Intern. Rev. Cytol. Vol. 21, 353-364).

The rabies virus 20 is deposited outside the motor end plate 13 has to permeate the edges of the PE cells 13 to get into axon 12. The rabies virus 20 deposited inside the muscle fiber 18,19 has to multiply and/or travel to reach the motor end plate axonal receptors site by passing through the post synaptic myoneural clefts 14, inter synaptic cleft 15 and presynaptic membrane at the axon 17. It can get inside the axon 16.

There are reports of prolonged incubation period which can take months and 5- to 6 years after the history of rabid animal bite. There isn't one explanation for protracted incubation period. Any one of the following conditions could contribute to the lengthy incubation period:

The numbers of rabies virus deposited are small in number and they have to multiply to reach certain threshold level to spread. Due to various metabolic, immunological factors and mechanical forces, it may take years to develop critical concentration to reach which prolongs the incubation period.

The viruses are located too deep in the muscle fiber or tendon farther away from the nerve end organs. Studies show that the rabies virus can be retained in infected myocytes during intermediate or long incubation periods (Charlton, K M. and G. A. Casey. Experimental rabies in skunks: Oral, nasal, tracheal, and intestinal exposure. Can. J. compo Moo. 43: 168-172. 1979. Experimental rabies in skunks. Immunofluorescence, light, and electron microscopic studies. Lab. Invest. 41, 36-44.1979).

They may be trapped in the muscle sarcoplasmic reticulum and has to multiply to emerge to leak from this organelle.

The trauma caused by the animal bite may initiate an inflammatory reaction at the site of the rabies virus deposition which attacks the multiplying rabies virus and localized them for months and years which hold the viruses under control till they break loose.

The pH and metabolic process surrounding the site of the rabies virus area of the muscle and the tendon while contracting won't allow the virus to multiply and spread.

Movement of the muscles fibers is that the viruses are constantly pushed back in their journey towards the motor end plate (MEP).

They may be trapped under the connective tissue sheath of the muscle, the cellular surrounding, and these structures won't allow RV to multiply and/or spread to nearby nerve components.

They may be trapped in the muscle and tendon which don't have axonal connection which the rabies virus can't spread. It may take years to find an opportune connection to spread retrograde.

If they come in contact with the nerve axon end, they may not have enough concentration to travel the nerve ending and to cause the disease.

The end organs may not have receptors which will accept the RV and endocytosed inside the nerve endings to be transported to CNS. The nicotinic acetylcholine receptor (nAChR) is identified as the receptor for the rabies virus to get attached at the myoneural junction (Lentz. T. L. (1985). Rabies virus receptors. Trends in Neurological Sciences 8. 360-364.) similar to snake venom. Rabies virus antigen was detected at sites coexisting with the nAChR in infected cultured chick myotubes and mouse diaphragms in a suspension of the rabies virus. It is thought that the distribution of the viral antigen detected by fluorescent antibody staining at these sites in neuromuscular junctions corresponded to the distribution of nAChR. There may be other receptors at the MEP that prevents or doesn't facilitate the entry of RV inside the axon which delays the internalization and the transfer-transport of RV.

The Genome of the RV is that it takes forever to multiply and to spread by the use of glycoprotein. The makeup of the glycoprotein is such that it won't allow the rabies virus to enter the axons.

The neurotubules at the MEP may be lacking to conduct the RV to the CNS when the virus enters the axonal terminal.

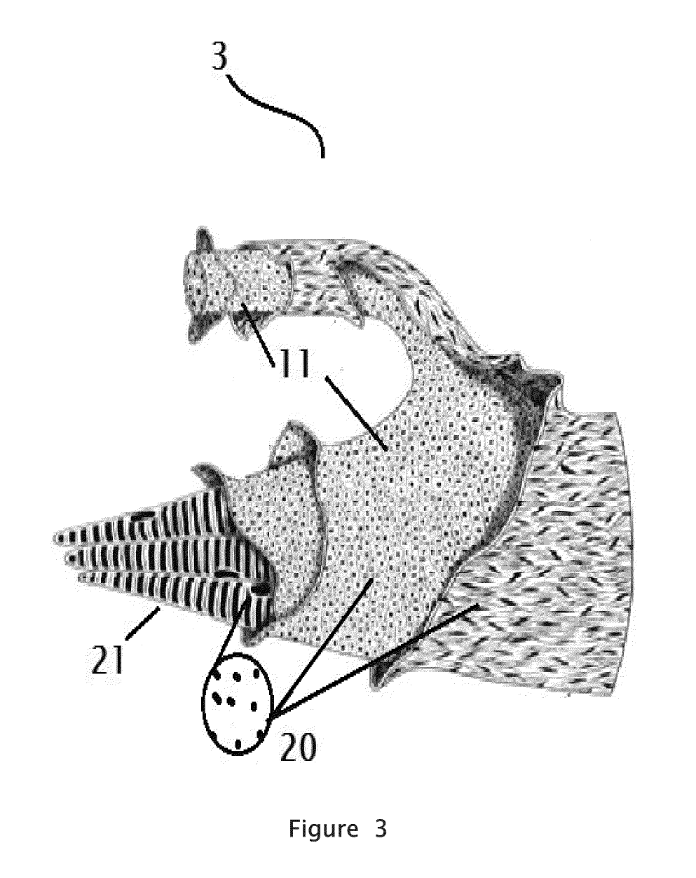

FIG. 3 is the drawing of the muscle spindle 3 showing that it is tightly enclosed in a connective tissue sheath (epineurium) and Perineural epithelium 11 and the rabies virus 20 has to pass through this tight covering to get into the intrafusal 21 nerve fibers. The Perineural epithelium tightly bounds the muscle spindle 3 and if this covering is damaged, the virus 20 get into the muscle spindle nerve endings to be transported retrograde to the CNS (from Shantha et al. Acta anat. 69: 632-646 (1968)).

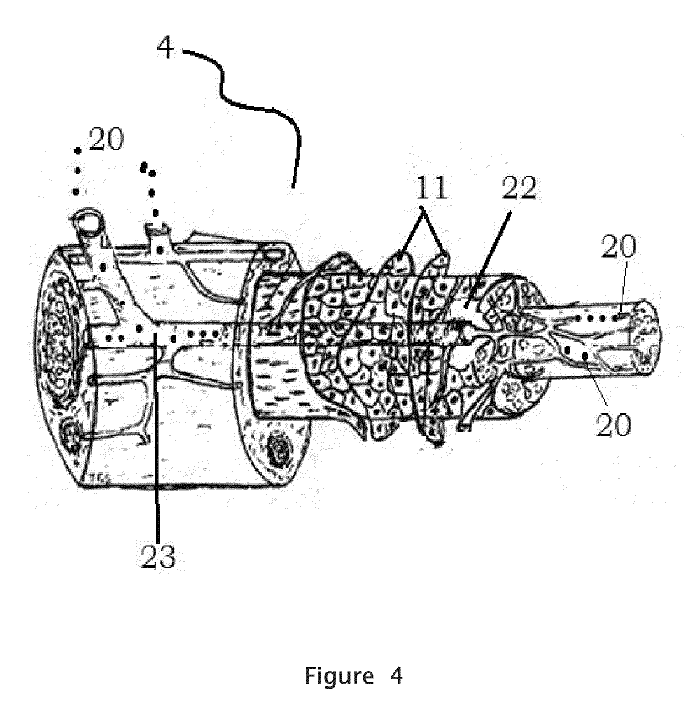

FIG. 4 is the drawing of the nerve fasciculi 4 showing the structure of the peripheral nerve fasciculi, its coverings, blood vessels, and the mechanism of transfer of the rabies virus 20 inside the nerve fasciculi to be transported retrograde to the CNS by the axons. If rabies virus enters the blood vessels 23, they are carried inside the nerve fasciculi to be deposited between the axons in the endoneurium. If the viruses are deposited in the Perineural epithelium 11, they are transported to inside the nerve fasciculi through the Virchow Robin space 22 which extends around the BV penetrating the nerve fasciculi into the core and the mantle layers of the nerve fasciculi (after Shantha, ASRA March-April Supplement, 1992). Once inside the nerve fasciculus in the edoneural surroundings, the RV can enter the axons at two sites:

1. The virus can enter the unmyelinated small axons surrounded by Schwann cells without myelin;

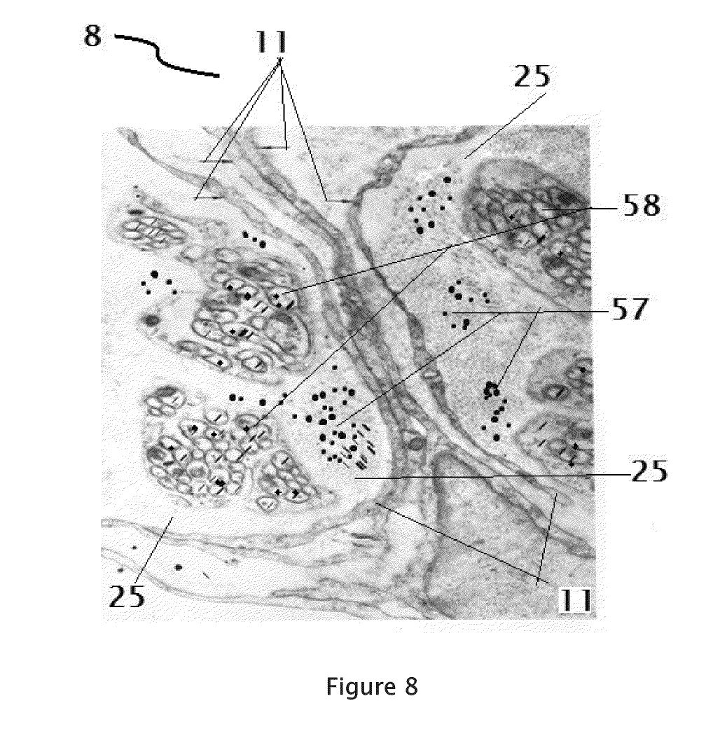

2. The rabies virus can enter only through the Node of Ranvier, which is metabolically active lacking insulating myelin. The myelin sheath surrounding the axon is impermeable to bacteria and viruses. It isn't known where the RV enters the Node of Ranvier. There may be receptors at the site which binds with rabies virus and enters the inside the axon. It is likely that the free floating and dividing virus enters the axons through the node of Ranvier by adsorptive endocytosis which doesn't require the participation of cellular metabolic active processes or receptors. It is possible that the RV is engulfed at this site by invagination of the separated Schwann cell lamellae at the node of Ranvier with high mitochondrial content. The same is true of the unmyelinated nerve bundle (see FIG. 8) only covered by invagination of Schwann cell cytoplasm (Dales, S. & Pons, M. W. (1976) Penetration of influenza examined by means of virus aggregates. Virology 69, 278-286. Tsiang, H. (1979). Evidence for an intra axonal transport of fixed and street rabies virus. Journal of Neuropathology and Experimental Neurology 38, 286-297. Tsiang, H., Koulakoff, A., Bizzini, B. & Berwald-Netter, Y. (1983). Neurotropism of rabies virus. An in vitro study of neurons and glia. Journal of Neuropathology and Experimental Neurology 42, 439-452).

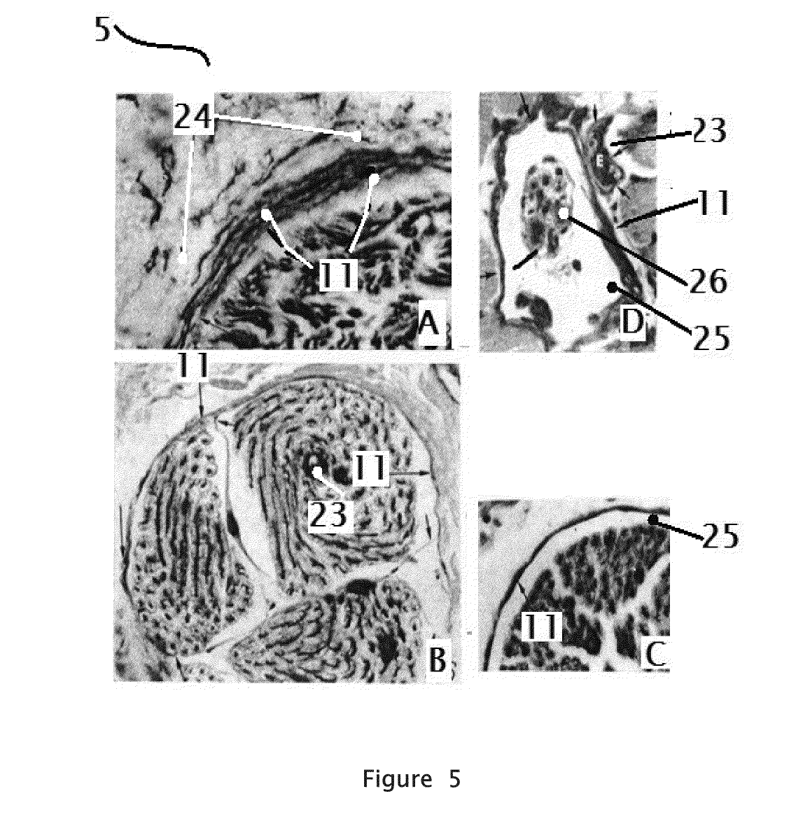

FIG. 5 is the stained histological transverse section of the nerve fasciculi 5 showing the structure of the peripheral nerve fasciculi surrounded by epineurium 24 and tightly enclosed Perineural epithelium 11 which is an impediment for the spread of RV deposited around the nerve trunk shown in A, B, C, and D. The Perineural epithelium 11 of the nerve fasciculi (ABC) and the muscle spindle D completely covers the nerve fasciculi. The RV can only enter the axons passing through the blood vessels 23 or through the Virchow Robin space 22 described in FIG. 4. The capillary extra cellular fluid in the sub Perineural space 25 can carry RV and act as a medium to transfer and to grow the RV inside the nerve fasciculi. (Shantha and Bourne, J Cell Biol. 1962, 14, 343-346. Nature 198, 607-608., Science 154, 1464-1467, Baer et al. WHO bulletin, 1965, 1968).

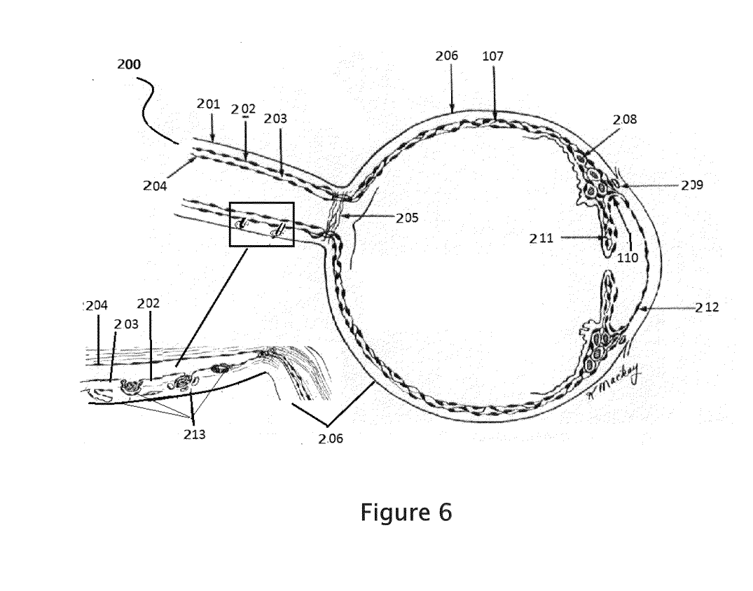

FIG. 6 is the drawing of the longitudinal section of the eyeball 200 which includes the optic nerve dura mater 201, leptomeninges 202, 204, and sclera 206, choroid, ciliary body, iris, and cornea are drawn to show their relationship with each other, trabecular meshwork, and aqueous humor circulation and their role in spread of rabies virus from the cornea. The dura mater 201 covering of the optic nerve continues with the sclera 206 and cornea. The Pia 204 and Arachnoid 202 mater forms the sub arachnoid space 203. When the cornea and conjunctiva are infected with RV, the virus gets into the naked nerve endings of the ophthalmic division of the trigeminal nerve to spread retrograde to the nuclei of the trigeminal never (as seen development of rabies after rabies virus infected cornea).

The RV is transported by the conjunctival and subconjunctival blood vessels to the episcleral and scleral BV. RV is transported to canal of Schlemm 209 where the RV spreads to the aqueous humor. It spreads to the corneal endothelium 212, ciliary body 208, choroid 207, Iris 211, their blood vessels and ciliary nerves, the ciliary ganglion, trigeminal nerve, into the CSF in the SAS 203 of the optic nerves. Some of the virus may escape through the arachnoid villi 213 and spread around the retro bulbar space which enters into all the nerve supply, BV of the eye ball, eye muscle, and retina. From the eye ball, the RV spread retrograde to the brain stem through cranial nerves III, IV, V, and VI.

The virus spreads from the CSF into the under surface of the cerebral cortex, brain stem, and the pituitary gland. The RV spreads to retina through the pigment layer or through the lamina cribrosa 205. The RV exits through the arachnoid villi formation 213 on the arachnoid mater of the optic nerve. When the CSF pressure rises due to rabies infection (or due to coughing and straining) of the brain, it is transmitted to the subarachnoid space which reflects the CSF pressure of the CNS can be transmitted rabies virus to the retina and choroid due to physical forces.

Pia and arachnoid (leptomeninges) covering of the optic nerve continue through the lamina cribrosa 205 as choroid with formation of supra choroidal and inter choroidal spaces 207. The choroid 207, an extension of the pia-arachnoid mater continues to cover ciliary muscle 108, non pigmented cells of the iris stroma 211, and various forms of trabecular meshwork 210 which drains the aqueous humor to iris-scleral angle, Canal of Schlemm 209, and corneal endothelium 212. Note: the arachnoid villi 213 projecting from the subarachnoid space into dura and close to BV. All these structures play a role in RV spread from the cornea (from Shantha T R and Bourne G H. Arachnoid villi in the optic nerve of man and monkey. Expt Eye Res 3:31-35 (1964). Acta Anat 61:379-398 (1965).

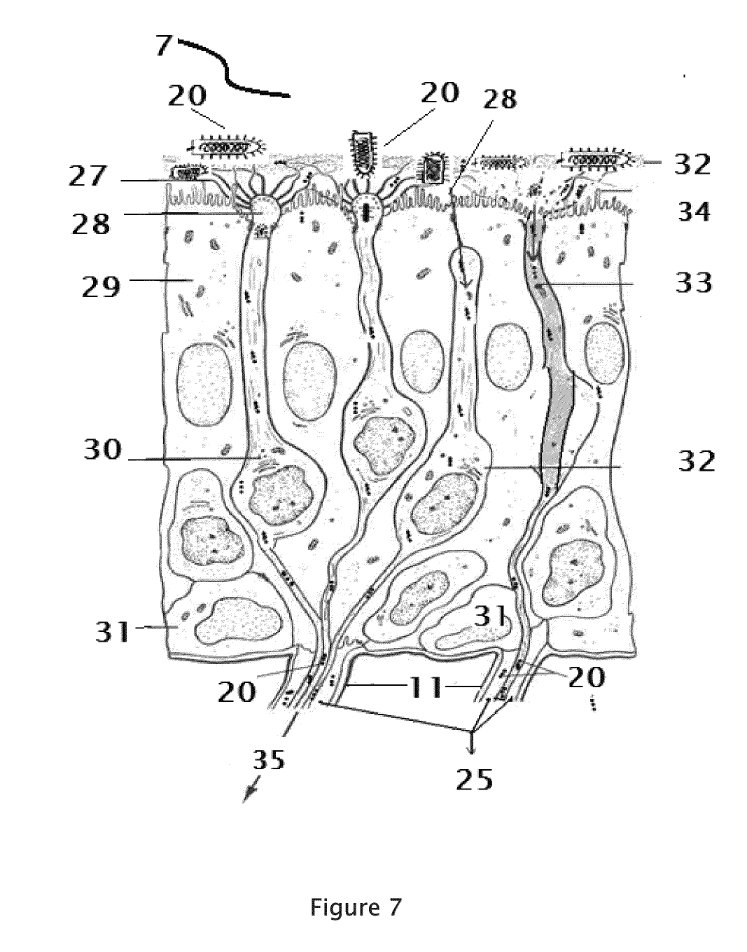

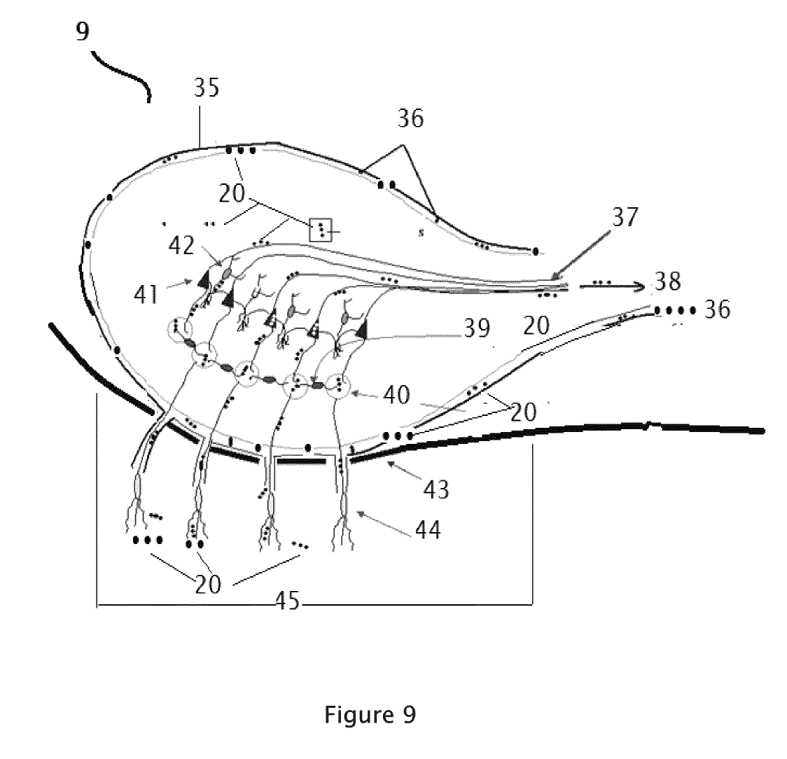

FIG. 7 is the drawing of the section of the olfactory mucosa 7 showing the route taken by the rabies virus 20 due to inhalation of the virus and its' route of transfer to the CNS. It shows how the RV 20 gets attached to the mucous film 32, entangled in olfactory cilia 27 of the olfactory cells and microvillus 34 of the supporting cells 29, and transported to through the olfactory axons 20, and Perineural epithelium 11 and sub Perineural space 25 to the olfactory bulb 35 and the SAS surrounding the olfactory bulb (FIG. 9). Note: The space created by dying olfactory cell 33, developing receptor cells 32, and their bulb 28 can easily transmit the RV 20 and therapeutic agents to the olfactory bulb and the rest of the CNS. The basal cells 31 transfer the RV 20 to the capillary space around the axons and to the sub Perineural space below the Perineural epithelium 25.

There are hundreds of olfactory cells dying at different locations. This creates a space between the olfactory cells which makes the olfactory membrane porous like sieve creating a route for the transfer of RV 20 and therapeutic agents to the CNS through the axons of olfactory bulb 35 and sub Perineural space 25 surrounding the axon bundle, where they enter the olfactory bulb through the cribriform plate of the ethmoid bone. (After Shantha and Nakajima. Z. Zellforsch. 103, 291-319 (1970).

FIG. 8 is the modified electron micrograph of the olfactory nerves showing the very small olfactory nerve axons 58 carrying the RV within their axons which travel retrograde to the olfactory bulb. This micrograph shows the Perineural epithelium 11 surrounding the olfactory nerve fasciculi with sub Perineural space 25 which also transmit the RV 57 directly to the CSF around the olfactory bulb and the brain SAS. (From Shantha and Bourne, Perineural Epithelium, in GH Bourne, Ed. In Structure and Function of Nervous Tissues. Volume I. Academic Press, New York. 1968. pp 379-459).

FIG. 9 is the drawing 9 of the longitudinal section of the olfactory bulb 35 showing the route taken by the rabies virus 20 by the inhalation transfer of RV to the CNS through the olfactory mucosa 45 to the olfactory bulb 35. The RV is transferred to the sub arachnoid space (SAS) 36 after passing through the olfactory mucosal nerve fasciculi. The RV 20 form receptor cells 44 pass through the axons through the cribriform plate of the ethmoid bone to join the olfactory bulb. From the olfactory receptor cell axons, they travel through the Glomeruli 40 to periglomerular cells 39, mitral cells 41, and granule cells 42, to olfactory tract 37, and reach the CNS 38. From the subarachnoid space 36 the RV spread all over the CNS to the rest of the body.

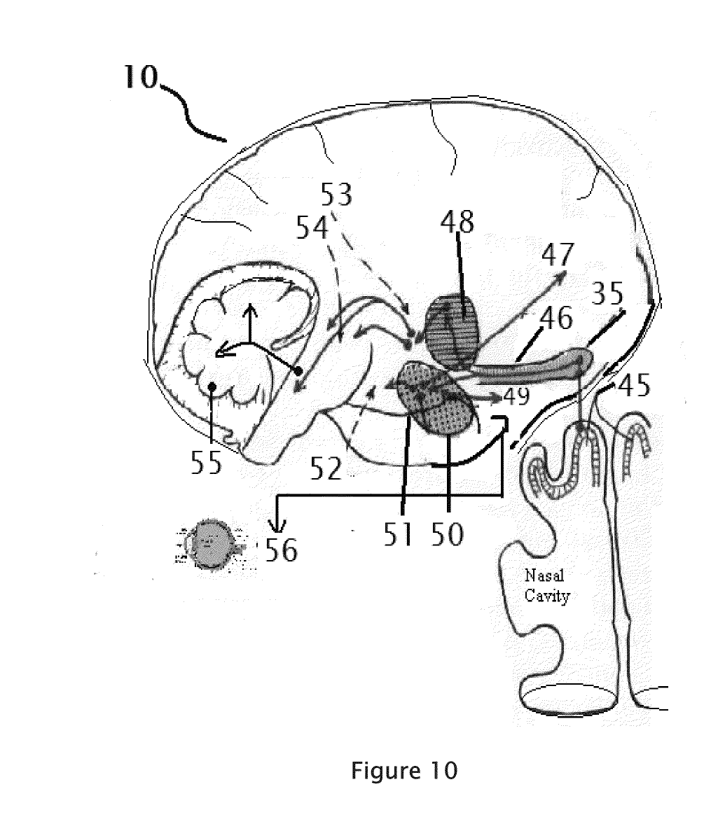

FIG. 10 is the drawing of the section of the olfactory bulb 10 showing the route taken by the rabies virus after inhalation inception from the olfactory mucosa 45 to the olfactory bulb 35 to the CNS through the olfactory tract 46. From the olfactory bulb, the virus spreads to the olfactory tract 46 to prefrontal cortex 47, medial olfactory area 48, to temporal lobe 50, to lateral olfactory area 51, hippocampus 52, hypothalamus 53, brain stem nuclei 54, to cerebellum 55. From the subarachnoid space surrounding the olfactory bulb 35, the RV spreads to the rest of CSF surrounding the frontal area of the undersurface of the brain, brain stem, and the eyes 56.

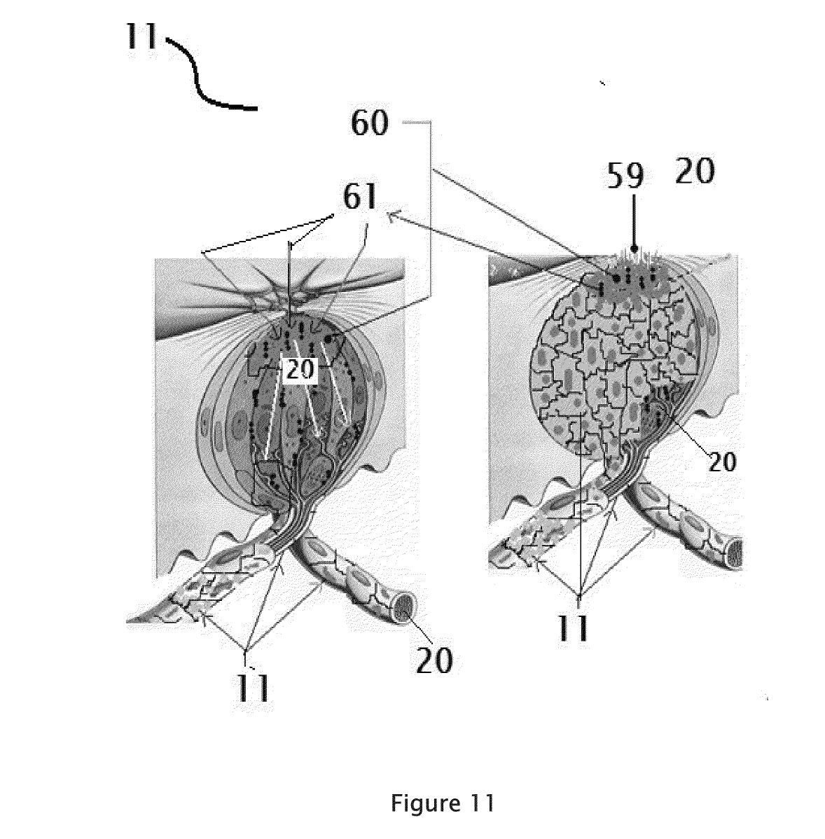

FIG. 11 is the drawing of the section of the taste buds 11 showing entry of RV into the axons of the taste bud nerve supply. The virus comes in contact with the taste bud with microvillus 59 which the virus binds to the polysaccharide complex 61 covering the taste bud apical part and the virus gets drawn into the pore 61 with the binding material which it gradually moves deeper. As it moves deeper, the RV 20 finds the free nerve endings at the base of the taste buds receptor cells where the RV gets attached to the axonal receptors or gets endocytosed. It spreads retrograde to the CNS brain stem nuclei. The RV travels below the Perineural epithelium 11 which it enters the CSF of the CNS. The taste buds are supplied by the lingual, glossopharyngeal, and vagus nerve (pharyngeal taste buds). Hence, from the taste buds, the virus can spread to the brain stem nuclei of these cranial nerves. The RV travels on to thalamus, taste area in the opercular-insular region.

It is important to note that the rabies virus entering the GI track can penetrate the intestinal villi, enter the parasympathetic, and sympathetic nerves (Auerbach's and Meissner's plexus), and spread retrograde to the sympathetic ganglion, dorsal root ganglion, lateral horn cells of the spinal cord which is the rest of the CNS (Shantha and Bourne. Zeitschritc fur Zellforschung 61:742-753 (1964).

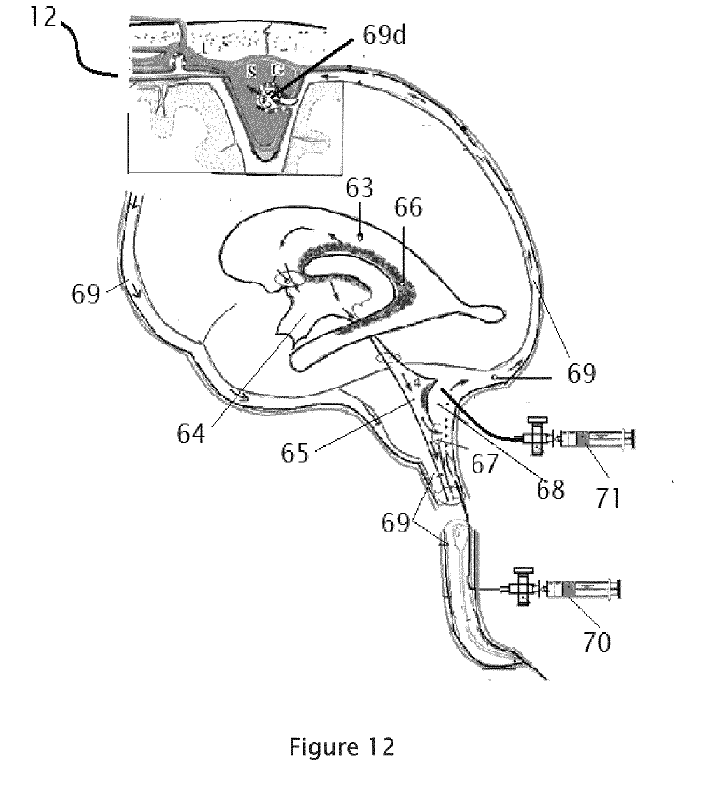

FIG. 12 is the drawing 12 showing the sites of production of CSF at lateral 63, third 64 and fourth 65 ventricles by choroid plexus 66 and its circulation through the central canal of spinal cord and drains through the three foramen on the roof of the fourth ventricle 67 (two openings situated one on each side of the fourth ventricle of the brain—foramen of Luschka, The median aperture is the foramen of Magendie in the mid line. From these openings, the CSF drains from the fourth ventricle into the cisterna magna—a large subarachnoid space) into cerebello medullary cistern 68, SAS 69 of the brain and spinal cord. It exits through the arachnoid villi 69 protruding into the sagittal sinus (SG) vein. Total volume of CSF in CNS is 140 ml which the ventricles contain 25 ml, SAS 115 ml. Choroid plexus 66 produces CSF at the rate of 0.2 to 0.7 ml/minute or 550-700 ml/day. Hence, the CSF is replaced 1.25 to 3.5 times a day. This is the reason that it is important to introduce therapeutic agents against rabies virus twice a day. The CSF pressure is between 5-15 mmHg. At about 11 centimeters of H₂O pressure which the rate of production and absorption of CSF are equal. The CSF acts as a major pathway for circulation of neuropeptides travelling from the brain to cranial and spinal nerves. It is important to analyze the CSF for rabies virus, cytology, and immunology during rabies diagnosis and treatment. We placed a permanent catheter in the SAS 70 or in the cisterna magna 68. This catheter 71 can be used for both for diagnostic and introducing the therapeutic agents. Through these SAS catheters 70 and 71, various antirabies therapeutic, pharmaceutical, biochemical, and biological agents or compounds with insulin are introduced intrathecal to the CSF in the SAS to diagnose and to treat rabies to clear the rabies virus from the surface of the brain and the spinal cord where the RV spreads through the cranial and peripheral nervous system.

Therapeutic agents have a barrier for entering the neuropile from SAS because the astroglia cells feet attach to the pia mater which acts as barrier: Pia-Brain Barrier. Surface area of the BBB is 5000 times larger than blood-CSF brinier which isn't that tightly controlled. From CSF and Pia, the therapeutic agents can penetrate up to 6 mm surface of the brain and spinal cord under normal circumstances. Some of the therapeutic agents can enter through Virchow-Robin space. Our method of the use of insulin injected into CSF with therapeutic agents will allow penetration into deeper depths of the brain (neuropile) to eradicate the offending agent from the brain, spinal cord, and proximal part of emerging cranial and spinal nerve roots and their ganglion.

Apart from the Para cellular (between cell junction) transport, there are several possibilities of transcellular transport to the brain such as passive lipophilic, adsorptive mediated, receptor mediated, carrier mediated transport in the SAS, Ependymal lining, and BBB. The spread of the therapeutic, pharmaceutical, biochemical, and biological agents or compounds into neuropile from SAS to treat rabies is enhanced by the use of our invention insulin as part of the therapy.

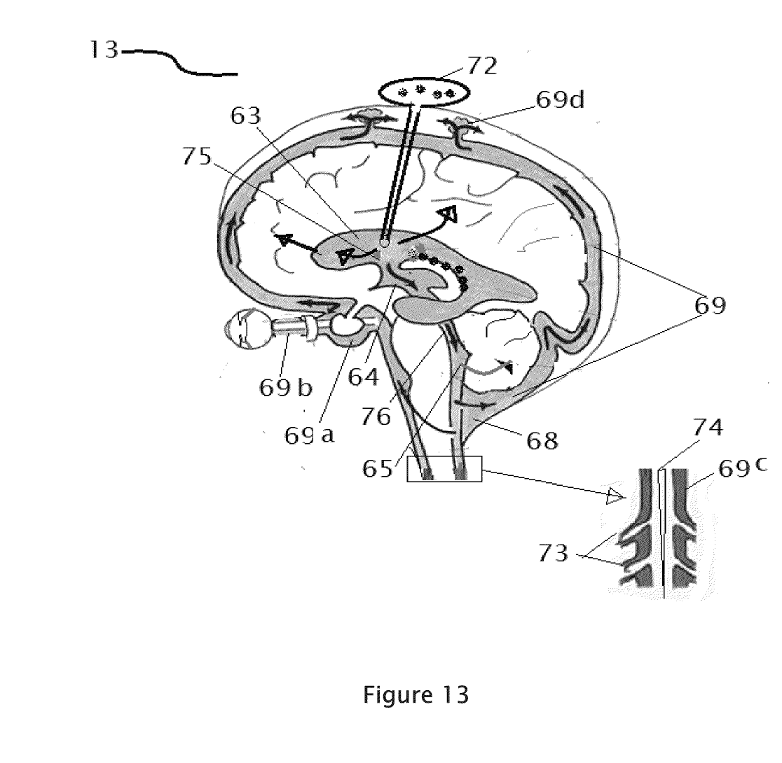

FIG. 13 is the drawing of the sites of the production of CSF at lateral 63, third 64, and fourth 65 ventricles by the choroid plexus. Its circulation through the interventricular canal 75 (foramen of Monro), aqueduct of Sylvius 76, central canal of spinal cord 74, drains through the three foramen on the roof of the fourth ventricle into cerebello medullary cistern (cisterna magna) 68 in the SAS, where it surrounds the brain 69, optic nerve 60b, and baths the pituitary gland 69a. CSF extends to the proximal part of the cranial and spinal nerve roots 73, which it surrounds the spinal cord 69c.

The insert shows the spinal cord with central canal 74, surrounded by SAS 69c, and note that the emerging spinal nerve roots 73 are surrounded by CSF. CSF from SAS exits through the arachnoid villi 69d into the sagittal sinus 69d, spinal nerve roots villi 73, and the optic nerve villi 69b. Ommaya reservoir with a catheter 72 is introduced to the lateral ventricle 63. The therapeutic, pharmaceutical, biochemical, and biological agents or compounds against the rabies virus are introduced to the lateral ventricles, where it circulates all over the brain and spinal cord into the neuropile which it acts against the rabies virus and its associated pathology. Hence, therapeutic agents introduced into the ventricles which circulate the agents through the CSF bathing the Ependymal lining of the ventricles, choroid plexus, optic nerve of the eye 69b, and central canal of the spinal cord 74, pituitary gland 68a, and the surface of the brain 69 bringing the therapeutic agents close to the afflicted brain which it can't be reached. Introduce therapeutic agents twice (or as needed) a day through the Ommaya reservoir that the concentration of the anti RV agents maintain constant concentration as CSF is absorbed through the brain surface.

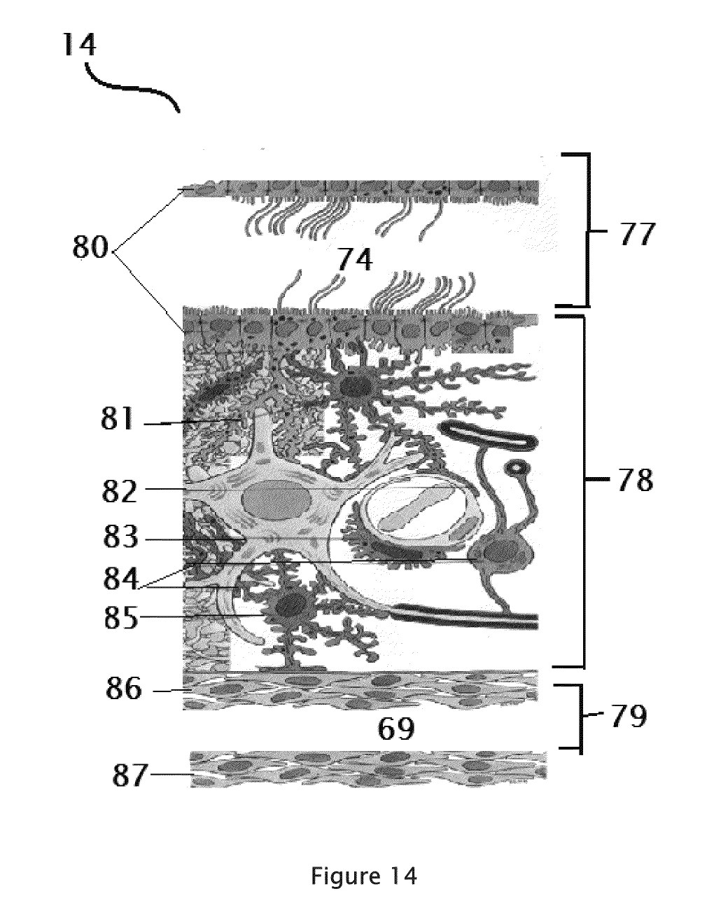

FIG. 14 is the drawing of the sites of action 14 of therapeutic agents introduced into the Ventricles 77 through Ommaya reservoir and SAS 79 catheter as described in the FIGS. 12 and 13, which will act on the RV, that are farther from the BBB blood vessels. They may not come in contact with the anti RV therapeutic agents in the neuropile 78. The therapeutic agents introduced through the Ommaya delivery system reach the Ependymal lining 80 where they are absorbed into the brain tissue (neuropile) adjacent to the central canal and the ventricles of the brain 74, 77. The therapeutic agents introduced through the SAS 69, 79 as described in FIG. 12 will circulate all over the brain surface which is enclosed by pia 87 and arachnoid mater 86. They permeate the pia mater 86 reaches the brain surface under it.

They travel along the Virchow Robin space in the deeper crevices of the brain. The therapeutic agents, against the RV, will reach the neuropile containing astroglia 81, and its end feet 82, oligodendroglia 84, pericapillary microglia 85, astrocytes 85, nerve cells, BV, nerve fibers with synapses from the Ventricle to the central canal of the brain; to the periphery of the brain and spinal cord. The therapeutic agents in CNS are brought as close as possible to the RV to act against the RV to eliminate the virus or to reduce the viral load, the spread of the therapeutic, pharmaceutical, biochemical and biological agents or compounds into neuropile from the ventricles and SAS to treat rabies is enhanced by the use of our invention insulin as part of the therapy (modified from Grays, Anatomy).

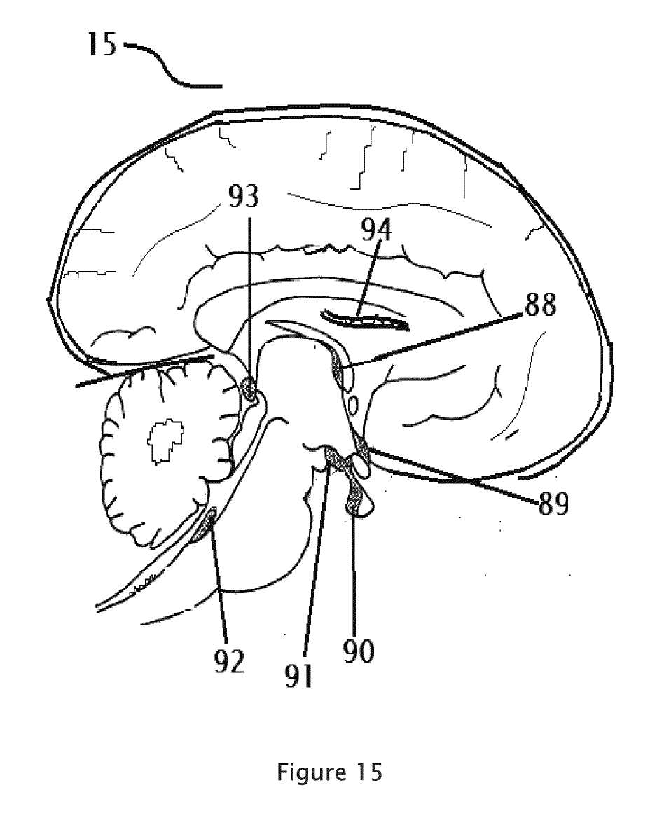

FIG. 15 is the drawing of the location of the circumventricular organs 15 which may play a role in hematogenous spread of the rabies virus and the therapeutic agents to CNS due to RV viremia and anti RV therapy introduce through the systemic circulation. There are several areas of the brain known as “circumventricular organs” where the BBB is weak and allows substances to cross into the brain and CSF freely with least impediment compared to the blood vessels with BBB within the neuropile of the CNS. The animal bite may activate lympho-haematogenous spread of the rabies virus to the CNS through nerve roots and circumventricular organs instead of classic axonal spread.

The circumventricular organs are where the RV and therapeutic agents can enter the CNS through the CSF and neuropile include: Pineal gland 93 which secretes melatonin and Associated with circadian rhythms and Neurohypophysis (posterior pituitary) that produces oxytocin and vasopressin into the blood to maintain BP and the urine output. Area postrema 92 a chemo sensitive vomiting center in the fourth ventricle of the brain stem and Subformical organ 88 is involved in the regulation of body fluids. Vascular organ of the lamina terminalis 89, a chemosensory area, detects peptides and other molecules. Median eminence 91 regulates the anterior pituitary through release of neurohormones. I would add choroid plexus 94, Ependymal lining of the ventricles and central canal, arachnoid villi, pia mater of the brain and spinal cord, and the emerging nerve roots of the CNS and Spinal cord (Shantha T R and Evans J A: Arachnoid Villi in the Spinal Cord, and Their Relationship to Epidural Anesthesia. Anesthesiology 37:543-557, 1972. Nakajima Y, Shantha T R and Bourne G H: Histological and Histochemical studies on the subformical organ of the squirrel monkey. Histochemie 14:149-160 (1968). Manocha and Shantha. Enzyme Histochemistry of the Nervous System (Macaca Mulatta, 1970, Academic Press, 18-305).

There aren't any reports that humans get RV brain infection through viremia in the above weak BBB areas of the brain. There is a theoretical possibility. Recent, study on animals does show that the RV viremia can spread directly to the brain without spreading or retrograde through the peripheral nerve axons (Preuss M R, et al. (2009). Hematogenous spread of rabies virus through Circumventricular organ. PL0S Pathog 5(6): 1000485.doi:10.1371/journal.ppat.1000485).

In the same fashion, the antirabies antibodies can get into the CSF through these circumventricular organs, besides Ependymal lining, pia linings, Virchow Robin space, arachnoid villi, nerve roots, blood vessels of Bates, nerve root lymphatic's, and the choroid plexus. The spread of the therapeutic, pharmaceutical, biochemical and biological agents or compounds into neuropile through these circumventricular organs to treat rabies is enhanced by the use of our invention insulin as part of the therapy.

It is important to note that the BV of the olfactory area and the eye ball are in direct communication with the cavernous venous plexus around the pituitary gland which communicate with the CNS at the neurovascular interface of the hypothalamus-hypophysis system and with complex venous sinuses within the cranium. From these sites, the rabies virus infection can spread from the OM or the cornea of the eye to the CNS through various weak BBB systems of circumventricular organs, linings, and venous network.

The possibility exists that the rabies virus passing the blood-brain-barrier via nicotinic acetylcholine receptor-mediated endocytosis into endothelial cells of brain capillaries through these blood vessel connections has been suggested.

The next possibility is that the invasion of rabies virus through circumventricular organ (CVO) (see FIG. 15), which are highly vascularized sites, that facilitate direct communication of neurons with blood and liquor through fenestrated endothelium. CVO either consist of neuronal cell bodies that sense various circulating substances (sensory CVO), or they are formed by Neurosecretory axons and glial cells (secretory CVO). Their special composition exposes them as targets for invasion of pathogens and trypanosome. (Schultzberg M, Ambatsis M, Samuelsson E B, Kristensson K, van Meirvenne N (1988) Spread of Trypanosoma brucei to the nervous system: early attack on circumventricular organs and sensory ganglia.) Neurosci Res 21: 56-61); as well as to other infectious agent microbes of all kind including rabies virus. The retrograde invasion of rabies virus from vessels from the OM and the eyes into the CNS through Neurosecretory fibers of the CVO of median eminence. The neuro hypophysis is strongly indicated, that after IV inoculation showed an almost exclusive involvement of the hypothalamic nuclei, where hormones for the regulation of the adenohypophysis are released (Mirjam A. R. Preuss et al 2009 IBID).

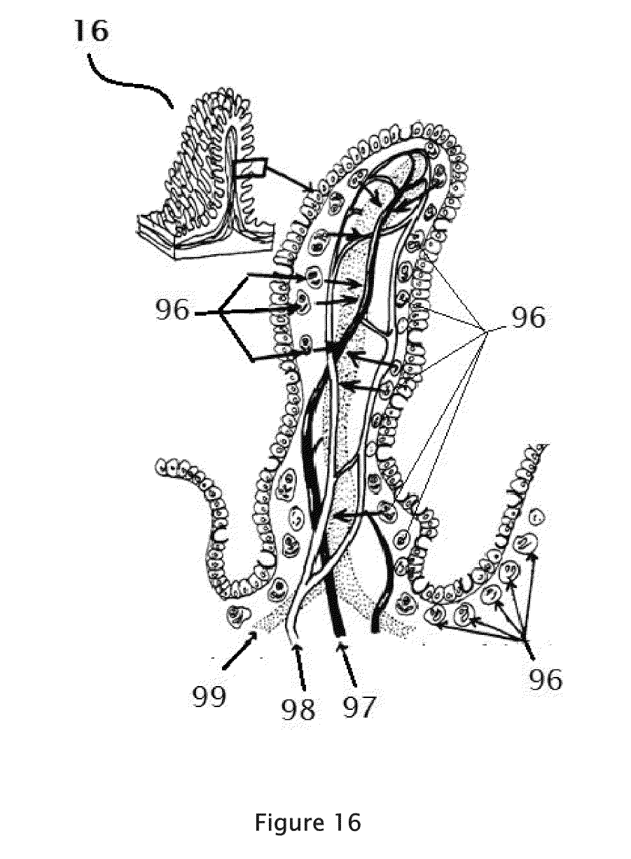

FIG. 16 is a section of a small intestine showing the villi 16 arrangement and magnified single villus with its epithelial cells lining. The contents of the villi 16, which absorb the nutrients and come in contact with infections from the lumen of the intestines. Note, immediately, below the lining of the villi is the lamina propia containing plasma cells 96 with close proximity to the artery 97, vein 98 and central lacteal (lymphatic duct) 99 which play an important role in production of immunity in rabies and other infections. They are located in the body tracts exposed to external environment like the respiratory, gastrointestinal, and genitourinary system (called the external secretory system). It is the first line of defense, where they pick up various antigens from food, bacteria, virus, and synthesize the immunoglobulins, which attack the invaders harmful to the body.

There are an estimated 180,000 plasma cells in the lamina propia per cubic millimeter of the intestines. When the RV enters lamina propia of the villi, these plasma cells pick up the RV protein which they synthesize antibodies against them. These ANA (immunoglobulins) are picked by the BV and lymphatic ducts 97, 98, 99, and circulate them all over the body. These immunoglobulins are too large to bypass the BBB of the CNS to enter the brain and to attack the RV. It is important to administer oral RV vaccine as soon as the diagnosis is made, if the virus hasn't spread to the salivary glands.

Once the RV is spread centrifugally, the anti rabies vaccine shouldn't be administered. Unless, the intradermal route is used as described in our invention. It is important to note that the antirabies neutralizing antibodies (ANA) are leaked through the circumventricular organs, choroid plexus of the ventricles, arachnoid villi of the sagittal sinus, optic nerve, dorsal root, ventral root arachnoid villi, Ependymal lining, cranial, dorsal, and ventral roots of the spinal cord. They circulate in the SAS. The spread of the ANA and the other therapeutic, pharmaceutical, biochemical and biological agents or compounds from SAS into neuropile to treat rabies is enhanced by the use our invention insulin as part of the therapy (diagram from Shantha, AIDS, A prescription for survival, International publishing house, 1991, page 119.)

There are millions and billions of preformed B lymphocytes and T lymphocytes in the lamina propia besides macrophages, and dendritic cells (FIG. 16). They are capable of forming highly specific antibodies (Humoral response by B cells). The T cells (cellular response) when activated by an appropriate antigen such as RV antigen are like molds, producing an exact replica capable of performing the particular function. Only the specific antigen that can react will activate them. For example, if a B lymphocyte is stimulated by a specific rabies virus antigen, the dormant clones of B cells will enlarge (lymphoblast). Each will divide rapidly forming about 400 mature plasma cells within four days. These plasma cells produce gamma globulin antibodies at a rate of 2000 molecules per second per cell.

These antibodies are secreted by the plasma cells in the lamina propria and lymphatic system which enter into the bloodstream. Some of them find their way to CSF. From blood, they are distributed all over the body where they seek antigens which have invaded the body. Unfortunately, the RV is sheltered by the BBB which these antibodies may not be able to reach the CNS in time with enough concentration contributing to death of these patients. This process of plasma cell activity continues for weeks until the plasma cells die. If the antigen continues to exist in the body, these dead plasma cells are replaced by new cells. Each B lymphocyte has about 100,000 antibody molecules. Each will react with only one specific type of antigen which is the rabies virus. When an appropriate antigen attaches to the antibody on the B cell membrane, it leads to the activation process.

The same happens in T lymphocytes. There are molecules similar to antibodies on T cells called surface receptor proteins (T cell markers). They become activated by the invasion of one specific activating antigen. By the time the immune system is activated which may take days, it is too late which the patient with rabies virus infection succumbs. To counter this delay in waiting for the immune response, which does arrive and not at the appropriate time, our invention of administering the RV human monoclonal antibodies (HMAB) directly into the CNS through the OM, SAS, IVB, IV, and IA with insulin, at the same time attempting to break the BBB that natural viral antibodies and HMAB reach the deep depths of the brain to clear the virus, preserve the brain from the onslaught of the virus and cure the disease.

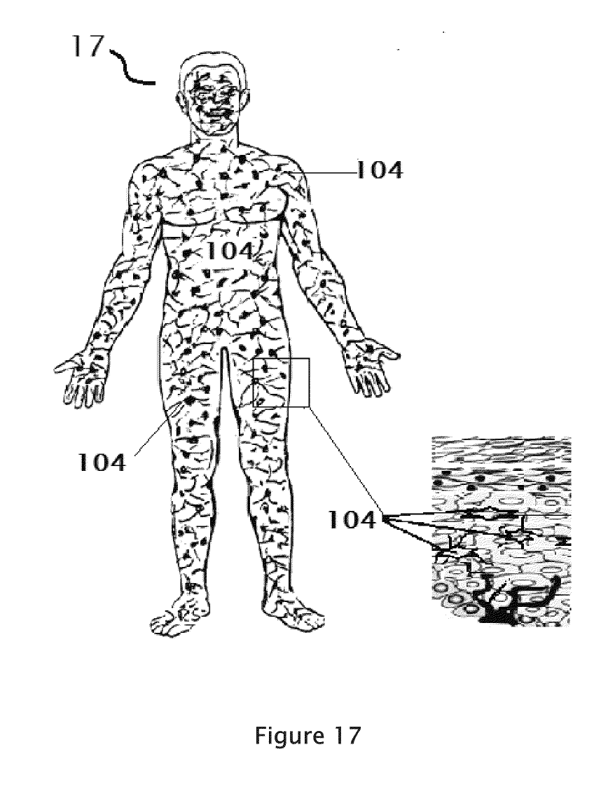

FIG. 17 diagram of the skin 17 being covered Langerhans immune defense cells 104 located in the epidermis. The Langerhans cells have processes like octopus spread in the skin epidermis like a fish net. These immune cells pick up antigens from skin due to infection or antigens introduced intra dermal as vaccine like antirabies virus vaccine. They pick up the antigen, process it, and transfer it to the neighboring dendritic immune system cells in the skin and to the adjacent lymph nodes. This processed antigen stimulates the T and B cells resulting in antibody production. The insert shows the location of Langerhans cells 104 in the keratinocyte layer (4^{th} layer of the skin), in between the basal layer in the bottom and granulocytes, stratum lucidum, and stratum corneum on the top. It is where we need to deposit PEP vaccines with insulin intradermally according to our invention (from Shantha, AIDS, A prescription for survival, International publishing house, 1991, page 184). Our inventive method describes the intradermal administration of PEP vaccine with insulin to develop rapid durable immunity against the rabies virus compared to the other methods of vaccinations.

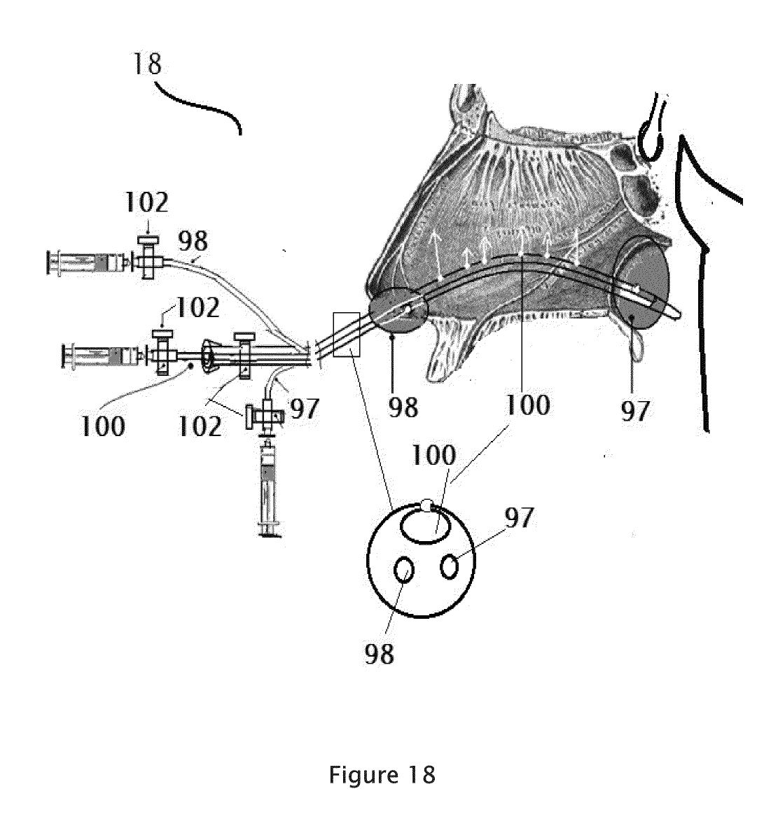

FIG. 18 is the diagrammatic presentation of the inventive device 18 to be used to deliver the therapeutic agents through the nasal cavity to the olfactory mucosal nerves, nasociliary nerve from the trigeminal ophthalmic division, nerve of the pterygoid canal, and nasoplatine nerve and the sphenopalatine ganglion. The device is made of three canulas 97, 98, and 100; connected to the proximal end by stop cocks 102 which attaches to the syringes. The canula 97 is connected to the distal balloon as shown in the diagram, and the 98 to proximal balloon, and the 100 to the drug delivery canula with multiple openings on the intranasal length which allows the therapeutic agents to be delivered to the proximity of the above mentioned nerve structures (olfactory nerves and other adjoining nerves). The therapeutic agents are absorbed by these neurological structures and transported to the CNS neuropile and to the CSF of the SAS without leaking back into nasopharynx and oropharynx.

First, place the patient in a supine position with head extended. Have suction catheter and equipment available if the patient needs to be suctioned in the nasal, oral, and pharyngeal areas. The catheter tip and the nostril are lubricated with KY jelly or other sterile Vaseline lubricants. If the patient is awake and agitated, local anesthetic spray such as Citanest spray or xylocalne jelly can be used to anesthetize the nasal surface sensory nerves. Then gently pass the catheter at the bottom of the nose touching the floor of the nose, directly at 90 degree angle to the external naris. As it passes towards the back, it comes in contact with the wall of the nasopharynx which can be felt as obstruction for further advancement. If one continues to push the catheter, it may appear the in the oropharynx. Then fill the distal balloon with saline or air. Then pull it gently forwards till it hits the posterior opening of the nose. Once it comes in contact, blow or fill the proximal balloon 98. Once you obtain a tight fit between these two balloons, the therapeutic agents can be administered gently through the syringe attached to the stopcock to the canula 100, and deliver the therapeutic agents to the nasal olfactory mucosa to cover the above mentioned nerve structures.

The capacity of each nasal cavity is estimated to be 7.5 ml. The practitioner may use be 3 to 3.5 ml to cover the olfactory mucosa in supine position with head extended. The balloons can be deflated and withdrawn after the therapeutic agents are absorbed from the olfactory area of the nose. This catheter prevents the drainage of the therapeutic agents back into the pharynx and prevents swallowing or entering the larynx. It helps to contain the therapeutic agents locally in the nasal cavity without the loss through the nasal choanal opening; i.e, that is the opening between the nasal cavity and the nasopharynx which prevents it from seeping into the pharyngeal opening of the pharyngo-tympanic tube. It is opening bound by: anteriorly and inferiorly by the horizontal plate of palatine bone, superiorly and posteriorly by the sphenoid bone and laterally by the medial pterygoid plates.

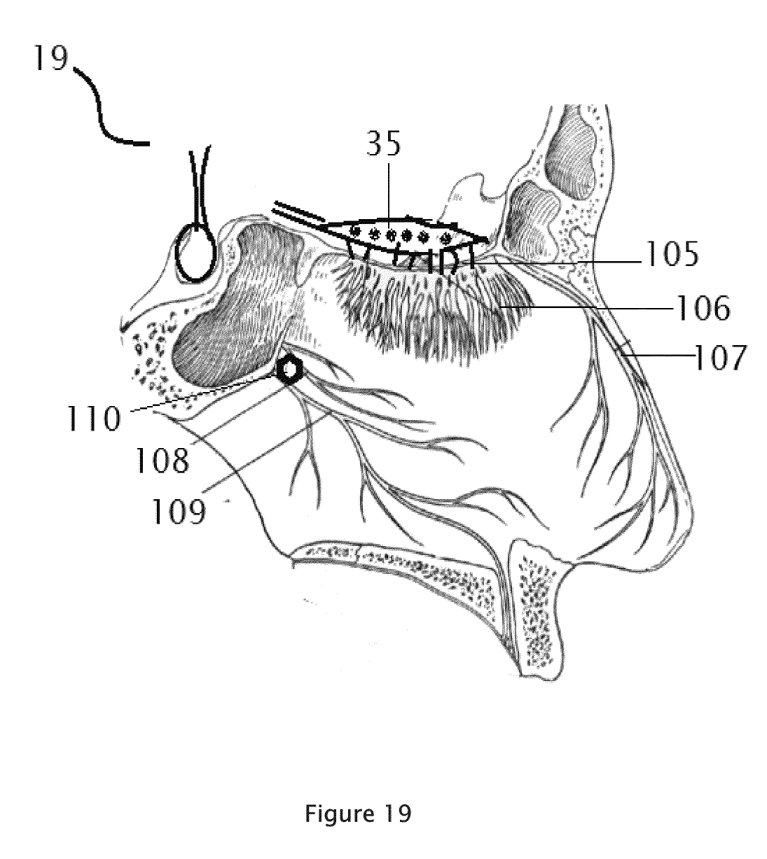

FIG. 19 is the diagram of the medial wall of the nasal cavity 19 and various nerve structures that the RV and therapeutic agents come in contact and transmitted to the CNS retrograde from the upper part of the nose from the olfactory area (OM). The RV and therapeutic agents with our invention insulin can pass through the olfactory bulb 35 conducted by the olfactory mucosa 106 and olfactory nerves 105. The RV and therapeutic agents are passed on to the CNS and the CSF through the trigeminal nerve branches that supply the nasal cavity. The RV and therapeutic agents come in contact with anterior ethmoidal nerve 107, nasoplatine nerve 109, medial, posterior and superior nasal branches 108 and the sphenopalatine ganglion 110. (Modified from Grays Anatomy.)

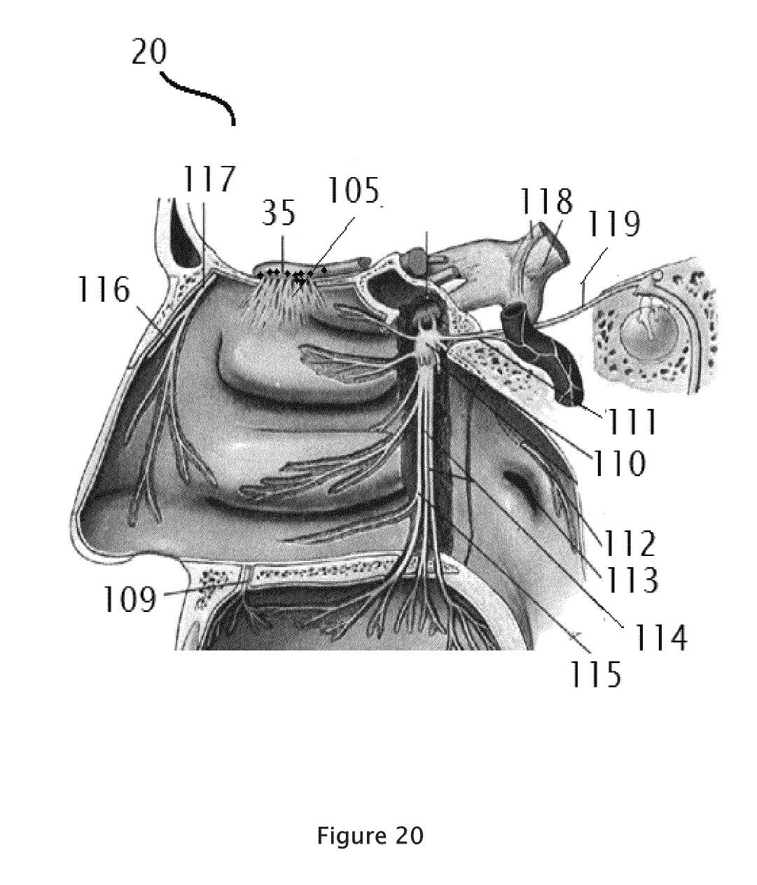

FIG. 20 is the diagram of the lateral wall of the nasal cavity 20 showing various nerve structures that the rabies virus (RV) and therapeutic agents with insulin come in contact and transmitted to the CNS retrograde. The RV and therapeutic agents can pass through the olfactory bulb 35 conducted by the olfactory mucosa and olfactory nerves 105. The RV and therapeutic agents are passed on to the CNS and the CSF through the trigeminal nerve 118, greater petrosal nerve 119, nerve of the pterygoid canal 111, pterygopalatine and pharyngeal nerve 112, lesser palatine nerve 114, greater palatine nerve 115, nasopalatine nerve 109, external nasal nerve 116, and the anterior ethmoidal nerve 117. The therapeutic agents can seep on to the middle ear through the pharyngeal opening of the pharyngo tympanic tube (Modified from Grays Anatomy.)

FIG. 21 is diagram of the brain capillary cross section 21 showing the components of the blood brain barrier (BBB) presentation of the capillary endothelial cells with tight junctions 215, investment of the outer layer of the capillary by astrocytes and the astrocytes end feet 216 and pericytes 217. In-between these cellular elements is the basement membrane 218 made up of amorphous non cellular elements, which prevent the leaking of the capillary contents, from inside to escape to the extracellular space 219. This basement membrane 218 binds the pericytes and the astroglial end feet from extracellular space cemented to the outer wall of the capillary endothelial cells of the BBB vessels of the CNS making a leak proof 400 miles barrier.

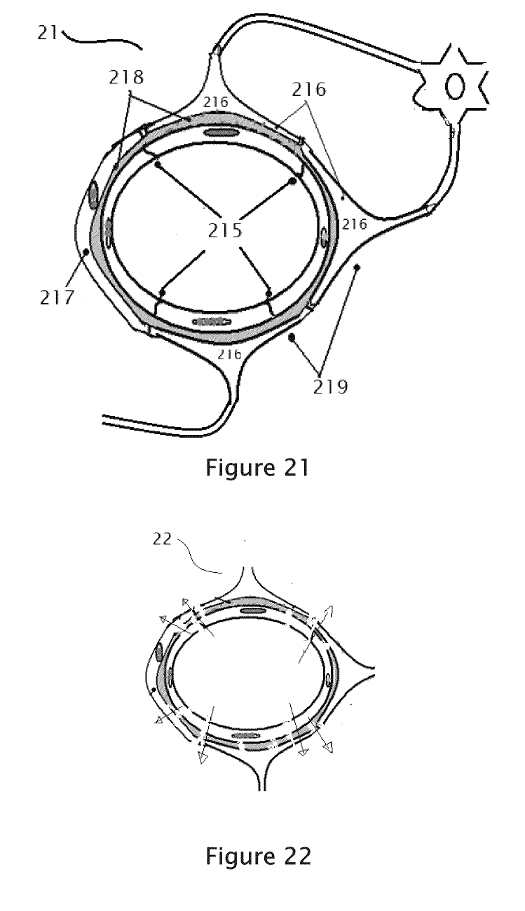

FIG. 22 is the diagram of the brain capillary cross section 22 similar to the FIG. 21 showing the leaking of the various cellular, solutes, rabies antibodies (ANS), and therapeutic agents from the circulating blood in these vessels to the extracellular space (multiple arrows) after breaking the BBB. The therapeutic, pharmaceutical, biochemical, and biological agents or compounds are administered parenteral, intra-arterial or intravenously to the neuropile from within the blood vessels of the blood brain barrier (BBB) after breaking the barrier using various methods described herein.

Besides passing directly to the brain through the olfactory system, the RV and antirabies virus therapies can spread from the pterygopalatine ganglion (sphenopalatine ganglion) through the sensory, motor, parasympathetic and sympathetic roots that it is connected. The possible roots involved in RV and therapeutic agents spread both centripetally and centrifugally are: Sympathetic root: Sympathetic efferent (postganglionic) fibers from the superior cervical ganglion travel through the carotid plexus, and through the deep petrosal nerve. The deep petrosal nerve joins with the greater petrosal nerve to form the nerve of the pterygoid canal, which enters the ganglion. Sensory root: Its sensory root is derived from two sphenopalatine branches of the maxillary nerve: their fibers pass directly into the palatine nerves and the Motor and the Parasympathetic root. Its motor root is derived from the nervus intermedius (a part of the facial nerve) through the greater petrosal nerve (parasympathetic). From this complex ganglion, the RV and its therapies can spread through the branches which supply the nose, soft palate, tonsils, uvula, roof of the mouth, upper lip and gums, and to the upper part of the pharynx. The lacrimal gland via the zygomatic nerve, a branch of the maxillary nerve (from the trigeminal nerve) connects with the lacrimal nerve (a branch of the ophthalmic nerve which is part of the trigeminal nerve) to arrive at the lacrimal gland.

Rabies Virus Receptors and Rabies Virus Reproduction

There are receptors needed for normal cell function and needed for the rabies virus to be attached to be transported far and wide. These receptors are hijacked by rabies virus to gain entry into cells by RV binding to these membrane-associated molecules which facilitate viral entry into the nerve cells and their processes. The rabies virus glycoprotein is to be of prime importance in this process. There is evidence of at least three rabies virus receptors and there may be additional ones. They are:

1. Nicotinic acetylcholine receptor of neuromuscular junction at peripheral sites needed for the RV to gain access to the CNS along peripheral nerves.

2. Neural cell adhesion molecule receptor found in neural cell which are susceptible cells for rabies infection.

3. Low-affinity p75 neurotropin receptor is a receptor for street rabies virus. There will be more receptors for the RV recognized besides the above named yet to be identified. They all play a role in inclusion and in transport of rabies virus.

After the rabies virus penetrates the axon, the virus sheds its membrane (covering), releases RNA and protein which travel to the cell body and their processes. The viral RNA generates messenger RNAs (transcription), which use the cell's machinery to produce the virus's five proteins (translation). The viral RNA creates copies of itself, which are assembled with the proteins into new microbes that emerge from the neuron's dendrites to attack the next nerve cell. The Ketamine inhibits the transcription phase of the rabies life cycle, which blocks the intracellular viral reproduction that does not kill the completely formed virus. The reproducible virus component is already in the cell body. This is one of the reasons that the ketamine hasn't worked to cure rabies. There are still the fully formed viruses which escape the ketamine, multiply, and spread.

Rabies Virus Spread from the CNS to the Perphery (Centrifugal Spread)

Centrifugal viral spread from the CNS to peripheral sites is a must for transmission of rabies virus to its natural hosts. Salivary gland RV infection is essential for the transfer of infectious oral fluids by rabid vectors. Salivary gland epithelial cells spread are a result RV spread along nerve fiber axons which are along the seeping CSF with the sub Perineural epithelial space and in-between the nerve axonal filaments within the nerve fasciculi. Ultra structural studies showed that the RVs were present in the basal region salivary gland acinal cells. The virus buds at the apical plasma membrane into the acinar lumen, intercellular canaliculi, and even to the membranes of secretory granules, and enters the saliva.

The RV from the CNS spreads to the parotid gland through the sympathetic, parasympathetic and through the cranial nerves. They pass through the sympathetic supply from the plexus on the external carotid artery, the parasympathetic secretomotor nerves through the tympanic branch of the glossopharyngeal nerve. They are relayed in the otic ganglion where they travel via the auriculotemporal nerve to the parotid gland. The RV can travel to the human parotid gland through the secretomotor fibers and the chorda tympani. RV travels to the submandibular gland through the submandibular ganglion, which it receives fibers from the chorda tympani of the facial nerve, the lingual branch of the mandibular nerve and the sympathetic trunk. The RV travel to the sublingual salivary glands through the lingual and chorda tympani nerves, and from the sympathetic nerves. These viruses take a circuitous route to reach the salivary glands. They are secreted with saliva to spread the RV to the victim. In the same fashion, the rabies virus spreads peripherally to the rest of the structures in the body described below.

RV infection involves neurons in a variety of extra neural organs, including the adrenal medulla, cardiac ganglia, plexuses in the luminal gastrointestinal tract, major salivary glands. Liver and exocrine pancreas, epithelium of the tongue, cardiac and skeletal muscle, hair follicles, and pancreatic islets causes myocarditis in human some cases of rabies.

Type of Clinical Rabies; Signs and Symptoms

There are two types of clinically different rabies cases reported. 1. Furious or encephalitic form of the disease (80% of the cases); 2. Paralytic or dumb form (20% of the cases). This form can be mistaken or mimics Guillain-Barre syndrome and other CNS afflictions.

Non-specific prodromal symptoms in rabies, including fever, chills, malaise, depression, fatigue, low energy, sleeplessness, anorexia, headache, anxiety and irritability may last up to 10 days prior to the onset of neurologic symptoms (Warrell, D. A. 1976. The clinical picture of rabies in man. Transactions of the Royal Society of Tropical Medicine and Hygiene 70, 188-195). About 30-70% of patients develop pain, paresthesias, and/or purities at or close to the site of the bite. The bite wound has often healed by the time these symptoms develop. Tremor has been described involving the bitten extremity. These local neurologic symptoms may be more common with bat rabies virus variants than with dog rabies virus variants. The initial neurologic symptoms may occasionally occur at a site distant from the bite, although, the pathogenic basis for this phenomenon is not clear. Two patients bitten on their toes developed rabies with early severe itching of their ears (Hemachudha, T. (1994). Human rabies: clinical aspects, pathogenesis and potential therapy. In: Lyssaviruses (C. E. Rupprecht, B. Dietzschold and H. Koprowski, eds). pp. 121-143. Berlin: Springer-Verlag).

Encephalitic or Furious Rabies:

This is the form that afflicts 80% of the cases associated with generalized arousal or hyper excitability, separated by lucid periods: Intermittent episodes confusion, hallucinations, agitation and aggressive behavior lasting almost 5 minutes; Biting behavior; fever; signs of autonomic dysfunction, includes excessive salivation, lacrimation, sweating, piloerection (goose skin) and dilated pupils due to the infection directly involving the autonomic nervous system centers or pathways in the hypothalamus, spinal cord and/or autonomic ganglia. Parasympathetic stimulation causes excessive production of saliva (more than 1 liter in 24 hours). Opened mouth, seizures, opisthotonus, and Priapism may occur. Cranial nerve signs such as opthalmoplegia, facial weakness, impaired swallowing and tongue weakness may be present.

There may also be nuchal rigidity, reflecting pia-arachnoid mater inflammation. About 50-80% of patients develop hydrophobia, “fear of water” which is a specific manifestation of rabies. On attempts to swallow, they experience contractions of the respiratory muscles, with epigastric pain. There may be retching, vomiting, coughing, aspiration, grimacing, convulsions, and hypoxia. Patients may die during severe spasms with the development of cardio-respiratory arrest if supportive care measures are not initiated (Warrell, D. A. and Warrell, M. J. (1991). Rabies. In: Infections of the Central Nervous System (H. P. Lamben, ed.). pp. 317-328. Philadelphia: B. C. Decker Inc. Warrell, D. A., Davidson, N. M., Pope, 1 I. M. et al. (1976). Pathophysiologic studies in human rabies. American Journal of Medicine 60, 180-190). The patients develop feeling of terror without being linked to pain with the mention of water or its sound can bring the horror. Patients avoid drinking for long periods of time, in the face of intense thirst, resulting in dehydration. A draft of air on the skin or the breath of an examiner may have the same effect (aerophobia). Patients ultimately, succumb to the CNS effects infection.

Paralytic or dumb rabies: Seen in 20% of the cases, flaccid muscle weakness is noticed early in the course of the disease. Patients may be literally dumb or present as mute due to laryngeal muscle weakness or paresis. The term dumb rabies refers to the quieter clinical features associated with prominent weakness though the Patients are alert. The Muscle fasciculation's and weakness usually begins in the lower extremity and spreads to the other extremities. It can be associated with bilateral deafness. There may be local pain, paresthesia, itching, or pruritus (due to C nerve fiber stimulation in the skin) at the site of the bite. This clinical picture can be easily confused with the Guillain-Barre syndrome or CNS and PNS afflictions. The urinary incontinence, and muscle swelling may be found. Many of the patients, who died of rabies, looked normal. Recent data indicates that the pathogenicity of a particular rabies virus strain is inversely proportional to its ability to induce apoptosis that the low-level apoptosis-inducing ability is associated with low anti-viral immune responses.

Natural rabies is in general characterized by severe neurologic signs and fatal outcome with relatively mild neuropathologic changes in the brain. A variety of experimental studies in rabies virus infection have been investigated for possible abnormalities in neurotransmission involving acetylcholine, serotonin and amino-butyric acid (GABA). Abnormalities of uncertain significance were found, but no fundamental defect was demonstrated that explains neuronal dysfunction in rabies that results in death.

Dysfunction of ion channels has been shown in rabies virus-infected cultures. The infection reduced the functional expression of voltage-dependent sodium channels and inward rectifier potassium channels with a decreased resting membrane potential reflecting membrane depolarization. There was no change in the expression of delayed rectifier potassium channels, indicating that nonselective dysfunction of ion channels had not occurred. The reduction in sodium channels and inward rectifier potassium channels could prevent infected neurons from firing action potentials and generating synaptic potentials, resulting in functional impairment. Nitric oxide neurotoxicity may mediate neuronal dysfunction in rabies. Induction of inducible nitric oxide synthase mRNA and increased brain levels of nitric oxide have been demonstrated in rabies virus-infected rodents. The significance of these findings is uncertain. The role of nitric oxide in rabies pathogenesis needs further study.

Neurotropic rabies viruses may cause cell death in the brain by either apoptosis or necrosis. Apoptosis is the result of synthesis of macromolecules and requires energy, whereas, necrosis is associated with energy failure. Each of these forms of cell death is associated with characteristic morphologic features. There are reports that there is down regulation of 90% of the genes in normal brain and only 1.4% of genes became up regulated including the genes involved in regulation of cell metabolism, protein synthesis, and growth, and differentiation in mice brain infected with fixed rabies virus. I am not certain that there is enough time in the human brain to initiate these genetic changes. If it is found, it is not that important to cause early death with almost intact brain. However, the neuronal cell death is not prominent in natural rabies, and, a greater understanding of the pathophysiology of the neurons and their dysfunction that occurs in natural rabies is needed.

Death typically follows within 2-4 weeks. Survival following rabies infection of the CNS has been reported in only ten human cases (Willoughby R E Jr, Tieves K S, Hoffman G M, Ghanayem N S, Amlie-Lefond C M, Schwabe M J, et al. Survival after treatment of rabies with induction of coma. N Engl J Med 2005; 352:2508-2514. Jackson A. C. Update on rabies diagnosis and treatment in Central nervous system, 2009, 296-3001, Jackson and Wunner Edited, RABIES, Second edition, Elsevier Academic press, 2007, 325-329). According to the latest CDC report, so far there are 10 recorded cases of rabies survival (Personal communication June 2009, Dr. Charles Rupprecht, Chief of Rabies, CDC of Atlanta).

The treatment of rabies involves

1. Post exposure Prophylaxis (PEP);

2. Treatment of the full blown disease and the;

3. Treatment of residual effects of the disease of the survivors (rehabilitation of recovering from the disease). The main steps in rabies affliction include deposition of the RV, replication, and/or spread along peripheral nerves to the spinal cord and the brain, dissemination within the CNS and finally centrifugal spreads especially to the salivary glands, and skin from the CNS along nerve routes to various organs, CSF, BV.

The prophylaxis of the infected animal bite and bat bite are discussed in detail by CDC of Atlanta on their web site. In this invention, we want to concentrate the RV in the CNS and eliminate the virus from the CNS and PNS, prevent the damage, and death of the afflicted. To understand our invention, knowledge of how RV spreads is important. Ultra structural studies in a skunk show that most the viral budding occurs on synaptic or adjacent plasma membranes of dendrites, with less prominent budding from the plasma membrane of the perikaryon. It is estimated that each cubic millimeter of cerebral cortex contains roughly one billion synapses (Alonso-Nanclares L, Gonzalez-Soriano J, Rodriguez J R, DeFelipe J (2008). “Gender differences in human cortical synaptic density”. Proc Nat Acad Sci U.S.A. 105 (38): 14615-9).

Most virions were found in part engulfed by an invaginated membrane of an adjacent axon terminal (synaptic) indicating transneuronal dendro-axonal transfer of virus. Virions were occasionally observed budding freely into the intercellular space (Charlton. K. M. and Casey. G. A. (1979). Experimental rabies in skunks: Immunofluorescence and electron microscopic studies. Laboratory Investigation 41. 36-44).

The axonal transport of rabies virus (CVS-challenge virus strain of fixed virus) has been studied in differentiated rat embryonic dorsal root ganglion cells. It was found that the attachment of rabies virus to neuronal extensions and virus production by infected neurons. Rabies virus demonstrated high binding affinity to unmyelinated neurites. This again supports our description that the RV is or may not be able to penetrate the thick myelin of large axons. It has to find the node of Ranvier to enter inside the axon.

How and why do Rabies Patients Die in Spite of all the Attempts to Save their Lives?

It is important to know how and the reasons that these patients die from rabies which we keep the patients alive till the clearing of the rabies virus from the CNS to save them from death and to decrease a residual disability. It is known that many of the patient's brain who died from rabies show no specific and non region orientated lesion in the brain. The brain almost looked normal without much damage to cause death. When all the patients die after several weeks in the intensive care, the virus can no longer be found in their bodies. Rabies virus, apparently, hijacks the brain into killing the body without much damage to the brain. Most of RV infection causes only restricted gross or histo-pathological lesions with presence of Negri bodies in the brains of human rabies patients despite the severe clinical neurological signs of rabies resulting in death. Negri bodies are eosinophilic, delineated cytoplasmic inclusions in certain nerve cells containing the virus of rabies. A pathognomonic inclusion bodies (2-10 μm in diameter) found in especially the Ammon's horn of the hippocampus which may, also, be found in the cerebellar cortex of the postmortem brain of rabies victims.

Once inside the cortex, the virus spreads rapidly in the highly interconnected brain through neuroskeleton network and neurotubules of the neuronal network. Rabies virus shows predilection to cerebellum concerned with body movements, the hippocampus involved in short term memory, and the limbic system which regulates emotions (where Negri bodies are found most of the time). With kindling of the immune system with the lapse of time, the virus is cleared from the brain by the immune system by ANA and the offending agent which is not found in the brain.

Even though considerable progress has been made in identifying elements of RV that play a role in the pathogenesis of rabies; it is still unclear, which host cell factors are not involved in the disease process nor are the mechanisms, that these factors determine the outcome of the disease. Even now, the immune recognition and the activation in response to RV infection aren't well understood. Its effect in the CNS is too late due to difficulty of immune mediated attack elements in crossing the BBB (FIGS. 21, 22) and paucity of lymphatic tissue in the CNS.

I believe that the rabies patients die mostly due to intense autonomic (both parasympathetic and sympathetic) nervous system stimulation of the CNS due to viral invasion of these autonomic nerve centers in the brain stem. The term “adrenergic” is derived from “adrenaline” which explains the hormones or drugs whose effects are similar to those of epinephrine produced from the adrenal glands. Adrenergic and parasympathetic stress is mediated by stimulation of adrenergic-parasympathetic receptors. The activation of post-receptor pathways from the CNS is due to rabies activated neuronal pathology.

Rabies illness is a potent stimulus of the autonomic nervous system and all the symptoms point in that direction. It is undisputable that the adrenergic-driven “fight-flight response” is a physiological reaction allowing humans to survive during evolution. However, in critical illness such as in rabies, sepsis, bleeding, severe trauma etc, results in an overshooting stimulation of the sympathetic and parasympathetic nervous system which may well exceed in time and scope in its beneficial effects.

Comparable to the overwhelming immune response during rabies, sepsis, etc; adrenergic stress in critical illness may get out of control and cause adverse effects on several end organs. The heart, lungs, blood vessels, salivary glands, and lacrimal glands seem to be most susceptible to sympathetic overstimulation in rabies. Detrimental effects like fever, impaired diastolic function of the ventricles, tachyarrhythmia, myocardial ischemia, striking apoptosis, and necrosis of the myocardium, respiratory and CNS plays a role at the end.

Adverse catecholamine effects have been observed in other organs such as the lungs (pulmonary edema, acute respiratory distress syndrome, elevated pulmonary arterial pressures), the coagulation (hypercoaguability, thrombus formation), gastrointestinal (hypo perfusion, inhibition of peristalsis), endocrinological (decreased prolactin, thyroid and growth hormone secretion), immune systems (immunomodulation, stimulation of bacterial growth), and metabolism (increase in cell energy expenditure, fever, hyperglycemia, catabolism, lipolysis, hyperlactatemia, electrolyte changes), bone marrow (anemia), and skeletal muscles (apoptosis). The intense stimulation of the parasympathetic stimulation results in the excessive salivation (more than a liter a day in some of the rabies patients), tears production, and profuse sweating with potential therapeutic options to reduce excessive adrenergic stress comprises temperature and heart rate control.

The adequate use of sedative/analgesic drugs (coma induction is not needed in rabies patients), aiming for reasonable cardiovascular targets, adequate fluid therapy, use of levosimendan, hydrocortisone or supplementary arginine vasopressin, air way management, and supplementary oxygen after intubation or through the nasal canula, and the use of nutritional support of the CNS using biopterin.

Our invention involves use of Insulin in combination with various anti rabies, antiviral, neuro protective, cardio-pulmonary protective, nutritional therapeutic agents, with hibernation, and brain cooling methods to eliminate this deadly infection, and to save the insult on the CNS, which the CNS mediates action on the rest of the organ systems.

Antirabies neutralizing antibodies (ANA) in the CSF: How do they reach the SUBARACHNOID SPACE (SAS) to get into CSF? Our invention to augment the delivery of ANA to the SAS and neuropile to Cure rabies

The presence of Antirabies Neutralizing Antibodies (ANA) in the CSF and the brain plays an important role in survival of the rabies patients. The ten cases that lived through rabies had high titers of ANA in their blood and in the CSF. Though the blood levels of ANA are high, their level in the CSF is much less. Analysis by Watson et al shows higher rabies antibody titers in survivors than non survivors. This observation is compelling which suggests a quantitatively higher Humoral immune response against the rabies virus in survivors (Watson et al., 2007 immune responses after rabies infection. Arch. Neurol. 64, 1355-1356).

In this invention we want to raise the ANA to eliminate the rabies virus from the CNS and the periphery by delivering HMAB and stimulating the natural ANA output. We can facilitate their entry by breaking the BBB (FIGS. 21, 22) and by use of OM, SAS, IVB, IV, carotid arterial route (IA).

Our study reported the mechanisms involved in the spread of epidural anesthetic spread to the subarachnoid space (SAS) can be applied in the delivery of ANA and HMAB from the periphery to the SAS, CSF, ultimately, to the neuropile. This study showed that there is a direct communication between dorsal and ventral roots through the Arachnoid villi in the epidural space where the sub Perineural epithelial space and the vascular system, and lymphatic's to the SAS which act as a route of transmission of the epidural administered therapeutic agents (Shantha T R and Evans J A: Arachnoid Villi in the Spinal Cord, and Their Relationship to Epidural Anesthesia. Anesthesiology 37:543-557, 1972). Hence, the antirabies neutralizing antibodies (ANA) produced by the immune system from the intestinal and lymphatic system (FIG. 6) reach the SAS and CSF in the above routes in the following manner:

Through the spinal and cranial nerve roots through the arachnoid villi in the cerebral sinus, spinal nerve roots and optic nerve of the eye leaks the antibodies to CSF

ANA can spread through the Batson venous plexus, or Batson veins to the subarachnoid space and CSF. Batson venous plexus are a rich network of valve-less veins in the human body that connect the deep pelvic veins, retroperitoneal veins, and the thoracic veins to the internal vertebral venous plexuses extending all the way into the cranial cavity. Their location and the lack of valves are believed to provide a route for the spread of cancer metastases to the vertebral column or brain. It has been shown that the Urinary tract infections like pyelonephritis spread to cause osteomyelitis of the vertebrae via this route. (Batson O V. (1940). “The function of the vertebral veins and their role in the spread of metastasis”. Annals of Surgery 112 (1): 138-49. Oneç B; Oksüzo\check{g}lu B; Hatipo\check{g}lu H G; Oneç K; Azak A; Zengin N (2007 July). “Cavernous sinus syndrome caused by metastatic colon carcinoma”. Clinical Colorectal Cancer 6 (8): 593-6). In the same fashion, these valves less Batson plexus of veins conduct the ANA to the CSF. The valve less vascular connection between the pelvic, abdominal, thoracic, cranial-vertebral venous plexuses (Batson s plexus of veins), the BV of the nerve roots and meninges, to the SAS and CSF are routes taken by the rabies virus antibodies to enter the CSF.

Ependymal lining of the ventricles and central canal CSF content leaking the ANA (FIG. 14).

ANA seep out of Choroid plexus of the ventricles (FIGS. 12, 13).

Seepage of ANA through the Circumventricular organs (FIG. 15) of the brain which do not have the type of BBB found inside the brain BV.

Transfer of ANA through the Virchow Robin space of the CNS and PNS (FIG. 4).

Transfer of the ANA through the complex lymphatic system from periphery all the way to SAS through the emerging nerve roots and blood vessels. Lymphatic channel connections from the gastrointestinal lamina propria, lymph channel connection from periphery of the CNS all the way to the SAS through the nerve roots and their blood supply and villi may carry the ANA to the subarachnoid space through the complex lymph channels and the vascular connections.

The ANA and other therapeutic agents have to pass through the BBB to reach inside the neuropile. The BBB is a formidable barrier but can be breached. And last of all ANA and therapeutic agents can pass through the leaking BBB due to rabies encephalitis, which can cause vasculities of the BBB capillaries making it incompetent. Administration of Insulin and other BBB breaking therapeutic agents described here allows more ANA and HMAB with other therapeutic agents inside the brain and leak into CSF.

In spite of all these routes of spread, it takes a long time for ANA to reach the SAS and the neuropile where there is paucity of ANA in these areas at the site of pathology compared to systemic levels. Hence, by using the inventive methods described herein, the ANA and other therapeutic agents such as HMAB can be delivered to the site of pathology including CSF to eliminate the virus and save the patients.

Use of Insulin as Therapeutic Agents Enhancer (Augmentation-Amplification Effect) in Our Invention for Treating Rabies

Before explanation and description of the disclosed embodiments of the present invention in detail, it is to be understood that the invention is not limited in its application to the details of the particular examples and arrangement shown. The invention is capable of other examples and embodiments in treating other oculopathies. The terminology used herein is for the purpose of description and not of limitation. As earlier enumerated above and recited below: This application has been filed in order to disclose. Insulin and Insulin-like Growth factor (IGF-1) have been found to have high therapeutic activity against rabies and many diseases including diabetes. Insulin and/or IGF-I not only restores the proper physiological functioning of the CNS. It enhances the effectiveness (augmentation-amplification effects) of other therapeutic, pharmaceutical, biochemical, and biological agents or compounds used in the treatment of rabies and other neurological diseases.

At present, the insulin is used to treat type I and some cases of type II diabetes. Our discoveries and inventions describes its use topically (locally) on the nasal olfactory mucosa (OM) by injecting into to the subarachnoid space (intrathecal) through a spinal needle, through a continuous subarachnoid delivery catheter (SAS), intravenously (IV) or intra arterially (IA), inter ventricular system of the brain using the Ommaya reservoir (IVB). The insulin can be used orally as liposomes or vitamin B12 or other methods (ORR) with various therapeutic, pharmaceutical, biochemical, and biological agents or compounds to treat rabies. Insulin has biological effects on healthy and disease afflicted cells. Its role in the uptake and augmentation-amplification effects of therapeutic, pharmaceutical, biochemical and biological agents or compounds on the rabies virus and rabies virus afflicted cells are described herein.

Our discoveries and inventions describes insulin use locally such as on olfactory mucosa, conjunctival sac, urethra, ears, oral cavity etc. to treat neurological diseases and other disease conditions besides diabetes including dry eye syndrome, glaucoma, AMD, Retinitis Pigmentosa, prostate diseases, middle and inner ear afflictions, CNS diseases, hair loss, enhancing eye lashes, activating vaccines, cytokines, Lymphokine, monoclonal antibodies, activating local immune system at lymph nodes, enhancing, the local effects of chemotherapeutic agents, in treatment of autoimmune diseases, age related changes of the facial skin, healing of wounds, gum diseases, local infections and multiple local and systemic therapeutic applications.

Our Invention of Using Insulin and its Biological Effects on Healthy and Disease Afflicted Cells in Rabies: its Role in Uptake, Augmentation-Amplification Effects of Therapeutic, Pharmaceutical, Biochemical and Biological Agents or Compounds Used Against the Rabies Virus in the CNS are Described Herein.

A variety of carriers, adjuvant agents, absorption enhancers and facilitators, assist to get entry into the cell, potentiators of therapeutic action (augmentation-amplification effects), cell metabolic activity enhancers, cell multiplication enhancers, and other methods have been used to enhance the absorption and/or to potentiate the effect of therapeutic, pharmaceutical, biochemical, and biological agents or compounds administered to the patients for improving the physiological function and the treatment of diseases. A biological agent of my invention is insulin which we want to use insulin to treat rabies which is 100% fatal.

So far, there are no reports of using the insulin as therapeutic agent to treat localized diseases in an organ or tissue such as rabies or parentarily to treat systemic diseases other than diabetes. The present inventor is the first person to experiment and to use insulin locally for almost a decade to treat many kinds of diseases of various tissues and organs in the body including cancers, and diseases of the ear, prostate, teeth, gums, CNS, eyes, hair growth, and other such conditions with many known therapeutic, pharmaceutical, biochemical and biological agents or compounds.

In 1965 Sodi-Pollares et al for the first time used glucose-insulin-potassium (GIK) solutions to treat patients with acute myocardial infarction and found that GIK limited the infarct size, reduced ventricular ectopy, and improved survival (Sodi-Pollares D, Testelli M D, Fisleder B L. Effects of an intravenous infusion of a potassium-glucose-insulin solution on the electrocardiographic signs of myocardial infarction. Am J Cardiol. 1965, 5:166-81). Insulin benefits the post ischemic myocardium by stimulating pyruvate dehydrogenase activity, which in turn stimulates aerobic metabolism on cardiac and other tissue reperfused.

Exogenous insulin helps to reverse insulin resistance during cardiopulmonary bypass, which contributes to increased serum concentrations of free fatty acids and decreased myocardial uptake of glucose and increased myocardial function. Intravenous infusions of insulin after coronary artery bypass graft surgery (CABG) have been shown to decrease the levels of free fatty acids and increase myocardial uptake of glucose. Insulin added to antegrade and retrograde tepid (29° C.) blood cardioplegia during coronary artery bypass surgery has been shown to stimulate aerobic metabolism during reperfusion, preventing lactate release and improving left ventricular stroke work index (Svensson S, Svedjeholm R, Ekroth R. Trauma metabolism of the heart: uptake of substrates and effects of insulin early after cardiac operations. J Thorac Cardiovasc Surg. 1990, 99:1063-73. Rao V, Mississauga C N, Merrante F. Insulin cardioplegia for coronary bypass surgery [abstract]. Circulation. 1998, 98 (Suppl):I-612).

Insulin increases the glutathione synthesis by activating gamma-glutamyl-cysteine synthetase. Its metabolic effects which reduces both polymorphonuclear neutrophils adhesion to ROS (reactive oxygen species—can be effective in post perfusion adhesion of white blood cells to ROS with resultant cellular damage) and stimulated tyrosine phosphorylation. Reactive oxygen species (ROS) are reactive molecules that contain the oxygen atom which include oxygen ions and peroxides and can be either inorganic or organic. They are highly reactive due to the presence of unpaired valence shell electrons. Cells are able to defend themselves against ROS damage through the use of superoxide dismutases, catalases, lactoperoxidases, glutathione peroxidases and peroxiredoxins. CNS damage by rabies can be attributed to the high production of ROS and the brain may not be able to defend against ROS.

Our use of insulin will certainly overcome this in the treatment of rabies. Small molecule antioxidants such as ascorbic acid (vitamin C), tocopherol (vitamin E), uric acid, polyphenol antioxidants, and glutathione, also, play important roles as cellular antioxidants. H₂O₂ induced lipid peroxidation was greatly inhibited by insulin pretreatment which can be a great protector of the CNS in rabies. Insulin increased redox status by increasing intracellular glutathione (GSH) content in oxidized cells. These results show that GSH can reverse the effect of oxidation (oxidative free radical damage) on tyrosine kinase activation and phosphorylation which plays an important role in cell signaling, that confirms the antioxidant activity to insulin.

This is an indication that insulin plays a profound role in maintaining homeostasis, reduces the production of ROS. Its related damage to the CNS and to improve cellular physiological function. In addition, insulin augments-amplifies the effects of therapeutic agents such as HMAB when locally used as described in this invention to save the CNS and to save the failing heart due to the rabies infection. Most of the rabies patients succumb to heart failure which the insulin with other therapeutic agents as enumerated above and below can save the CNS and the failing heart, and give life back to rabies patients.

U.S. Pat. No. 2,145,869 by Dr. Donato Perez Garcia disclosed a method for the treatment of syphilis in general and neurosyphilis in particular using subcutaneous insulin injections to induce hypoglycemic shock. Then administer intravenously arsenic, mercury, and bismuth, therapeutic agents with glucose and calcium chloride resulting in increased crossing of the blood brain barrier (BBB—FIGS. 21, 22) by therapeutic agents to act against the spirochete which causes the neurosyphilis. It was never used on rabies which now we have the opportunity to use insulin in the rabies cases OM, SAS, IVB, IV, and IA.

U.S. Pat. No. 4,971,951 and U.S. Pat. No. 5,155,096 discloses Insulin Potentiation Therapy (IPT) for the treatment of virally related diseases such as herpes, AIDS, as well as Gonorrhea, duodenal ulcer, gall stones, epilepsy, schizophrenia, asthma, arthritis, osteomyelitis, herpes, cancers and many other disease conditions using insulin to deliver the drugs inside the cell with less or non-toxic low doses therapeutic agents.

None of these inventions describe the use of insulin and/or IGF-I through OM, SAS, IVB, IV, and IA to be delivered to disease afflicted condition of the brain including rabies. None of these inventors and patents discloses or describes the regional tissue or organ specific use of insulin and/or IGF-I in a restricted area of the tissue or organ to treat disease states as described here in for treating rabies afflicted brain. Using insulin with normal dose of therapeutic, pharmaceutical, biochemical, and biological agents or compounds like HMAB, ketamine, colchicine, vinblastine, procrit, progesterone, and neurotrophic factors effects the rabies afflicted brain can be augmented-amplified and help to relive the viral burden the CNS, preserve the brain, and cure the disease.

Physiologically, the insulin activates and participates in all the metabolic pathways in the normal, disease afflicted cells systemically, and locally which can lead to increased DNA, RNA, and protein synthesis that results in increased growth by mitosis (Osborne C K, et al. Hormone responsive human breast cancer in long-term tissue culture: effect of insulin. Proc Natl Acad Sci USA. 1976; 73: 4536-4540).

Insulin enhances the permeability of cell membranes to many therapeutic agents besides glucose, and electrolytes, which helps and facilitates to move the drugs and therapeutic agent molecules from extra cellular fluid (ECF) to intracellular fluid (ICF), that means from the outside of the cells to the inside of the cells. This has been demonstrated in its use in coronary artery bypass graft (CABG) surgery and in our studies of local effects of insulin by the inventor. The use of insulin with antirabies therapeutic agents will move them inside the cellular elements of the CNS (neuropile) and act against the rabies virus.

Insulin is an anabolic trophic hormone needed for the maintaining health, growth, multiplication, of all cells in the body including the healthy vascular endothelium, neurons in the brain and retina, hair cells in the cochlea and vestibular apparatus, olfactory receptor cells and other cells in the body. Increased cellular metabolic activity induced by insulin enhances the uptake and enhances the action of all therapeutic, pharmaceutical, biochemical and biological agents or compounds including HMAB by the cells and inside the cell including the cells responsible for affliction by rabies virus. Once inside the cells, the insulin augments and amplifies the effects of any and all therapeutic agents including the agent proven and/or approved to treat rabies and other neurological diseases such as Alzheimer's, Parkinson's, depression, MS, ALS etc. by restoring the physiological function.

In our decade of studies and medical practice and experimentation, we found that there is not a single disease except hypoglycemia induced by insulin, insulinomas or otherwise, which cannot be treated using Insulin to enhance the effectiveness of the therapeutic, pharmaceutical, biochemical and biological agents or compounds including the treatment of rabies.

In an ingenious vitro studies, it has been methodically and conclusively demonstrated that the Insulin activates and modifies metabolic pathways in MCF-7 human breast cancer cells, and increases the cytotoxic effect of methotrexate up to 10,000 (ten thousand) fold (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemother Pharmacol (1985) 15: 272-277). Research studies in human breast cancer, my own studies on every kind of cancer, and infection in any part of the body have shown that the group treated with insulin plus low dose methotrexate and other anticancer agents (and/or antibiotics for infection, autoimmune diseases treatments, monoclonal antibody treatment etc.) responded better than the patients treated with insulin or chemotherapy alone. Many of the patients were cured of the disease (Eduardo Lasalvia-Prisco et al. Insulin-induced enhancement of antitumoral response to methotrexate in breast cancer patients. Cancer Chemother Pharmacol (2004) 53: 220-224. Ayre S G, Perez Garcia y Belton D, Perez Garcia D Jr (1990) Neoadjuvant low-dose chemotherapy with Insulin in breast carcinomas. Eur J Cancer 26:1262-1263; T. R. Shantha presented at Cancun IPT meeting 2nd meeting 2004 and unpublished studies). These observations supports the findings of Alabastor (IBID) that disease or healthy cell and disease causing microbes sensitivity to the therapeutic and biological agents such as those to be used to treat rabies including ketamine, HMAB and others could be increased (augmentation-amplification effects) many times by using the method described in this invention using insulin and/or IGF-I.

We have used insulin locally as a therapeutic agent in chronic non-healing wounds, burns, after draining the hydrocele of the tunica virginals sac in the scrotum, periodontal disease, post surgical wound healing, delayed healing of broken bones, prostate, and bladder afflictions, teeth and gum afflictions, eye, ear diseases, and many other diseases which will be reported later. Rabies is a neurological local disease afflicting the brain which insulin can be used as therapeutic agents and as an augmentation-amplification effector of therapeutic, pharmaceutical, biochemical and biological agents or compounds used against this deadly RV with HMAB, amantadine, biopterin, platelet growth factors, procrit, progesterone, ketamine and other anti rabies viral therapeutic agents.

In an important experiment, Zheng et al showed the role of insulin like growth factor-I (IGF-I) which have insulin like effects; induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I can independently stimulate cells growth and promote the health of the cells which make the CNS especially after the destructive effects of rabies. (Shantha T. R., Unknown Health Risks of Inhaled Insulin. Life Extension, September 2007 pages 74-79, Post publication comments in September 2008 issue of Life Extension, Pages 24. Shantha T. R and Jessica G. Shantha Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January, 2009, Issue # 306, pages-106-110).

Insulin exerts trophic effect on the cell physiology without discriminating whether it is normal, metaplasic, dysplasic, heteroplasic or carcinogenic (Philpott M P, Sanders D A, Kealey T. Effects of insulin and insulin-like growth factors on cultured human hair follicles: IGF-I at physiologic. J Invest Dermatol 1994; 102: 857-61, Shantha IBID). It is a known physiological phenomenon that the insulin does bind to the receptor sites of the IGF-I and insulin receptor sites. The multiple profound physiological, pharmacological, therapeutic effects, induces cell growth (besides glucose transport), enhances metabolism, enhances mitosis, enhances (augmentation-amplification effects) the therapeutic effect of other pharmacological agents against the disease which causes the agents including microbial agents as reported (Shantha T. R., Life extension September 2007:74-79,) on the cell, that it binds has been reported in above publications. Any dysfunction of the CNS due to rabies can be restored back to normal using the described inventive methods.

Insulin, potassium, and glucose are routinely administered to treat low potassium levels in the cells to this day. The inventor has used this method to lower the potassium levels in the blood for 3 decades. Insulin and glucose facilitates the entry of potassium inside the cell—a life saving measure. Similarly, the Insulin deposited in the OM, SAS, IVB, IV, and IA will enhance the uptake of therapeutic, pharmaceutical, biochemical, and biological agents or compounds by the dysfunctional neuronal cells due to rabies described in this inventive method. The present, inventor has used insulin which it potentiates uptake and enhances the therapeutic action of diverse therapeutic agents to cure and/or curtail curable acute, chronic, and incurable diseases like cancer, Lyme disease, scleroderma, lupus, psoriasis, antibiotic resistant staphylococcus infection, MRSA infection, chronic wounds, neurological diseases, inner, and middle ear affliction, autoimmune diseases, leprosy, prostate pathologies, skin diseases, herpes zoster of the eye with antiviral agents, tuberculosis, and many other diseases with good results.

Inventors have used insulin with other specific treatment modalities against depression, Alzheimer's, Autism, Parkinson's and many other neurological diseases successfully. It needs to be delivered to the brain through proper routes (Shantha, T. R. Site Of Entry Of Rabies Virus Form The Nose And Oral Cavity; And New Method Of Treatment Using Olfactory Mucosa And By Breaking BBB, presented at The 2^{nd} International Rabies In Asia Conference Held In Hanoi, 2009, Pp 70-73, and The Rabies in the North Americus (XX RITA), held in Quebec City, 2009, Pp 20-21).

Insulin increases metabolic activity and enhances therapeutic agents' actions in all organs and cells. It can play an important role in treatment of many diseases including rabies (Shantha T. R.; 1. discovery of insulin and IPT: amazing history, 2. high dose methotrexate therapy using Insulin; 3 local injections of tumors with insulin and cytotoxic drugs; 4. two and three cycle insulin potentiation therapy: Presented at 2^{nd} international conference on Insulin potentiation Therapy held at Cancun, Mexico, Jun. 28-Jul. 1, 2004).

A synergy between certain membrane and metabolic effects of insulin on cell molecular biology increases therapeutic efficacy of all anti rabies therapeutic, pharmaceutical, biochemical and biological agents or compounds, It does reduces doses of the drugs, enhances their uptake, with augmentation-amplification effects greater than before therapeutic efficacy. The insulin enters the cells and increases the safety of therapeutic agents. The present inventive method enhances the uptake of therapeutic agents which enhances their therapeutic effect inside the cells of the disease. This causes the disease afflicted cells of rabies to cure the disease.

Our preliminary studies have shown that the OM unlike normal skin or conjunctival sac doesn't act as a barrier for entry of the insulin. This is due to the paucity or to the non existence of the reduced glutathione. The insulin with the anti rabies therapeutic agents deposits on the OM, SAS, IVB, IV, and IA which are rapidly absorbed and reaches the brain. Its intricacies to act against the RV where the RV is multiplying and creating neuronal dysfunction and damage.

In accordance one aspect of the invention are the therapeutic agents that fight against rabies with insulin are administered through the OM, SAS, IVB, IV, and IA which are mixed with well-suited vehicle or carrier. The compositions of this invention may comprise aqueous solutions such as e.g., physiological saline, oil, gels, patches, solutions or ointments. The vehicle which will carry these biologically active therapeutic agents may contain OM compatible preservatives such as e.g., benzalkonium chloride, surfactants like e.g., polysorbate 80, liposomes, or polymers. For example, methyl cellulose, polyvinyl alcohol, polyvinyl pyrrolidone, and hyaluronic acid etc. We do use sterile water or normal saline in our preparation.

There are various forms of insulin used to treat diabetes which can be formulated to be used in our invention. They are grouped under rapid, short, intermediate, and long acting insulin. It is dispensed as premixed form containing rapid to long acting insulin. Insulin products are categorized according to their putative action profiles as:

1. Rapid-acting: insulin lispro, insulin aspart, and insulin glulisine

2. Short-acting: regular (soluble) insulin

3. Intermediate-acting: NPH (isophane) insulin

4. Long-acting: insulin glargine and insulin detemir

The following table summarizes the time of onset; peak action and duration of action are summarized in the following table.

| | | | | |
|---|---|---|---|---|
| | | Peak | Effective | Maximum |
| Insulin | Onset | Action | duration of | duration |
| Preparation | of Action (h) | (h) | action (h) | (h) |

RAPID-ACTING ANALOGUES AND PREPARATIONS

| | | | | |
|---|---|---|---|---|
| IInsulin lispro | ¼-½ | ½-1¼ | 3-4 | 4-6 |
| (Humalog), | | | | |
| Insulin aspart | | | | |
| (NovoLog), Insulin | | | | |
| glulisine (Apidra) | | | | |

SHORT-ACTING

| | | | | |
|---|---|---|---|---|
| Regular (soluble) | ½-1 | 2-3 | 3-6 | 6-8 |

INTERMEDIATE-ACTING

| | | | | |
|---|---|---|---|---|
| NPH (isophane) | 2-4 | 6-10 | 10-16 | 14-18 |

LONG-ACTING ANALOGUE

| | | | | |
|---|---|---|---|---|
| Insulin glargine | 3-4 | 8-16 | 18-20 | 20-24 |
| (Lantus) | | | | |
| Insulin detemir | 3-4 | 6-8 | 14 | ~20 |
| (Levemir) | | | | |

We prefer to use rapid and intermediate acting insulin at OM, SAS, IV, IA and IVB sites in rabies cases.

Glucose concentrations in the blood are expressed as milligrams per deciliter (mg/dL or mg/100 mL) in the United States, Japan, Spain, France, Belgium, Egypt, and Colombia. The millimoles per liter (mmol/L or mM) are the units used in the rest of the world. Glucose concentrations expressed as mg/dL can be converted to mmol/L by dividing by 18.0 g/dmol (the molar mass of glucose). For example, a glucose concentration of 90 mg/dL is 5.0 mmol/L or 5.0 mM. During a 24 hour period blood plasma glucose levels are typically between 4-8 mmol/L (72 and 144 mg/dL). Although, 3.3 or 3.9 mmol/L (60 or 70 mg/dL) is referred to as the lower limit of normal glucose. The symptoms of hypoglycemia typically do not occur until 2.8 to 3.0 mmol/L (50 to 54 mg/dl). The precise level of glucose considered low enough to define hypoglycemia is dependent on (1) the measurement method, (2) the age of the person, (3) presence or absence of effects (symptoms), and (4) the purpose of the definition. The debate continues to what degree of hypoglycemia warrants medical evaluation or treatment, or can cause harm.

One has to bear in mind the possibility of developing hypoglycemia when the insulin is being used. Our use of insulin through the olfactory mucosa, intrathecally (SAS-CSF), and intra arterial injected directly to the brain did not develop the hypoglycemic effects. The typical threshold for hypoglycemia is 70 mg/dL (blood sugar level of 3.9 mmol/L). It may be higher or lower depending on a patient's individual blood glucose target range.

Generally, the hypoglycemia is defined as a serum glucose level (the amount of sugar or glucose in a person's blood) below 70 mg/dL. Symptoms of hypoglycemia in general appear at levels below 60 mg/dL. Some people may feel symptoms above this level. Blood glucose levels below 50 mg/dL affects the brain function. Signs and symptoms of hypoglycemia which includes erratic or rapid heartbeat, sweating, dizziness, confusion, unexplained fatigue, shakiness, hunger, feeling hot, difficulty in thinking, confusion, headache, seizures, and potential loss of consciousness.

If severe hypoglycemia develops, it should be treated with oral ingestion of a fast-acting carbohydrate which are glucose tablets, fruit juice, fruit bowl, chocolate bar, or regular Coca-Cola, sugary drinks, or eat plain sugar followed with a drink of water or IV administration of 25% glucose. It is important to test blood sugar 15 minutes after administration of insulin if hypoglycemia develops. The blood sugar can be monitored with the finger sticks.

There are several mechanisms which glucose and insulin protect the rabies damaged nerve cells and restores normal function by our inventive method. Glucose is the preferred substrate during periods of cell damage and ischemia. Adenosine triphosphate derived from glycolysis is vital for stabilization of membrane ion transport, which is crucial to the integrity, endothelium, nerve cell integrity, vascular smooth muscle cells, nerve fibers and their terminals. Preservation of the function in the CNS decreases any further damages by the rabies virus and participates in their repair. Glucose with the help of insulin esterifies intracellular free fatty acids which decreases their toxic end-products and oxygen free radicals.

Glucose is a direct precursor of pyruvate which is carboxylated to the citric acid cycle substrates malate and oxaloacetate. It can replenish depleted substrates, thus, stimulating oxidative aerobic metabolism with the help of insulin and preserves the normal functioning of the rabies afflicted cells. Glucose with the help of insulin esterifies intracellular free fatty acid which decreases their toxic end-products and oxygen free radicals. Our invention of insulin with glucose can make the neurons and its associated glial cells function better. Insulin will counter act the excitatory effect of glutamate on NMDA receptors. The ROS damage generated by the rabies infection and will cut down the neuronal damage.

Our principle method of treatment of rabies is the administration of using determined units of insulin to selected routes such as OM, SAS, IVB, IV, and IA routes, wait 10-15 minutes to take effect. The administration of therapeutic agent through the desired route, followed with infusion of intravenous glucose to bringing back the blood glucose level to the desired normal level. This basic principle is to be followed in all our protocols in treatment of rabies unless otherwise specified.

The deficiency of biopterin in rabies is blamed for neurological symptoms and damage. This is a consequence after recovery in one case (Willoughby IBID). The use of insulin through OM, SAS, IVB, IV, and IA will be described in our invention. This will enhance the uptake of the biopterin administered orally or parentarily, and will reverse the adverse effects on the CNS that is due to the rabies infection which depletes the brain of this brain nutrient.

The therapeutic pharmaceutical insulin preparation to be used for OM, SAS, IVB, IV, and IA may contain buffering ingredients like sodium chloride, sodium acetate, gluconate buffers, phosphates, bicarbonate, citrate, borate, and likewise.

The OM, SAS, and IVB administered antirabies therapeutic agents preparation may contain surfactants like polysorbate surfactants, polyoxyethylene surfactants (BASF Cremaphor), phosphonates, saponins and polyethoxylated castor oils. Preferably, the polyethoxylated castor oils are commercially available.

The antirabies pharmaceutical preparation including insulin to be used on the OM may contain wetting agents like the carboxymethylcellulose, hydroxypropyl methylcellulose, glycerin, mannitol, polyvinyl alcohol or hydroxyethylcellulose and the diluting agent, which may be water, distilled water, sterile water, or artificial tears. The wetting agent is present in an amount of about 0.001% to about 10%.

The OM, SAS, and IVB antirabies formulation with the insulin invention may include acids and bases to adjust the pH tonicity imparting agents like the sorbitol, glycerin, and dextrose. The other viscosity imparting agents such as sodium carboxymethylcellulose, polyvinylpyrrdidone, polyvinyl alcohol and other gums, suitable absorption enhancers, such as surfactants, bile acids, stabilizing agents such as antioxidants, like bisulfites and ascorbates, metal chelating agents, such as sodium EDTA, and drug solubility enhancers, like polyethylene glycols are used. These additional ingredients help make commercial solutions with stability that they need not be compounded.

OM, SAS, IVB, IV, and IA antirabies medications compositions of this invention (insulin and other combinations) will be formulated to be compatible with the olfactory mucosa, pia meter, and Ependymal lining of the brain. The preparation used in OM, SAS, IVB, IV, and IA should be isotonic with blood. The insulin and other therapeutic agents' compositions intended for direct application to the OM, SAS, IVB, IV, and IA will be formulated to have a pH and tonicity which are compatible with the CSF. This will normally require a buffer to maintain the pH of the composition at or near physiologic pH (i.e., pH 7.4) and may require a tonicity agent to bring the osmolality of the composition to a level at or near 210-320 millimoles per kilogram.

In the following detailed description of the invention, reference is made to the drawings, microphotographs and tables where reference numerals refer to like elements which are intended to show by way of illustration specific embodiments. The invention that we describe using insulin and IGF-1 with or without other known anti rabies therapeutic, pharmaceutical, biochemical, and biological agents or compounds enumerated may be prescribed and practiced. It is understood that other embodiments may be utilized and that structural changes may be made without departing from the scope and spirit of the invention described herein.

The antirabies compositions are used on the OM, SAS, IVB, IV, and IA routes of the CNS. The composition should be sterile in the form of an isotonic solution. The constitution may contain non-toxic supplementary substances like the emulsifying agents, wetting agents, and bodying agents.

Treatment of Rabies with Human Anti-Rabies Human Monoclonal Antibody (HMAB) with Insulin as Described in Our Invention

Our invention incorporates the use of HMAB, after, the administration of insulin through OM, SAS, IVB, IV, and IA routes. The U.S. Patent Application Publication Number: US 2009/0041777 A1 (by William D. Thomas, JR., Somerville, Mass. (US); Donna M. Ambrosino, Jamaica Plain, Mark, (US); Robert Mandell, Collins, Iowa (US); Susan Sloan, Watertown, Mark (US); Gregory J. Babcock, Marlborough, Mass. (US); Charles Rupprecht, Lawrenceville, Ga. (US) on Human Antibodies Against Rabies And Uses). Insulin provides a recombinant human anti-rabies monoclonal antibody (HMAB) that specifically binds to a broad variety of rabies virus isolates and inhibits the ability of the virus to infect cells.

These inventors have demonstrated the antibodies ability to neutralize (i.e., inhibit or block) the rabies virus in vitro (e.g., in a RFFIT assay). In another embodiment, they demonstrated that the antibodies have the ability to inhibit rabies virus infectivity in vivo in the animal or a human. These human monoclonal antibodies or antigen binding portions of the invention specifically binds to rabies virus G glycoprotein acting against the rabies virus. Human monoclonal antibodies of this invention can be prepared virtually unlimited amounts in highly purified form.

Accordingly, the antibodies are suitable for prognosing, diagnosing, and/or treating an individual exposed (post exposure prophylaxis—PEP) or suspected of having been exposed to rabies. This HMAB invention totally eliminates the need for a donor source of human anti-rabies serum immunoglobulin. It's associated with immediate and late complications. This is an improved method of passive immunotherapy for treating a subject infected with rabies virus in combination with other antirabies therapies with insulin described in this invention using OM, SAS, IVB, IV, and IA routes.

Our invention describes the use of these newly developed safe human MAB of the above invention through OM, SAS, IVB, IV, and IA routes with insulin to be delivered CNS and PNS with ANA bodies to stop the reproduction of the rabies virus which will kill the remaining rabies virus. Along with the HMAB against rabies virus, our invention involves using MAB against various cytokines (anti TNF antibodies—Etanercept or other similar embodiments) that are produced by the microglia and other immune system of the brain, these are an innate (nonspecific) defense reaction. A first line of the immune defense is an innate reaction which results in production of non specific cytokines affecting the CNS and its function. The MAB, Etanercept, can drastically reduce the inflammatory effect of these cytokines in the early stages of the rabies virus infection of the brain and maintain homeostasis in the CNS. Our inventive method allows them to be used through OM, SAS, IVB, IV and IA routes after breaking the BBB. They should be administered after the administration of short acting insulin to augment-amplify the effects HMAB and MAB against the rabies virus and cytokines described above.

The second response to rabies infection is Adaptive (specific) defense which develops in response to non specific innate response to early infection leading to specific reaction against the specific infection. The antibody response and the leukocyte-mediated response called the Humoral cell mediated responses which takes weeks and dispenses them to the site of rabies virus infection.

These naturally produced ANA which have difficulty getting inside the CNS. It is too late when they enter the CNS and the patient succumbs. This is important to use the recently developed HMAB through our inventive method of administering them with insulin, which is delivered through OM, SAS, and IVB routes, IV and IA after breaking the BBB to augment-amplify the effect of these HMAB, that are naturally produced ANA and save the lives of rabies patients.

Preparation of the Rabies Patients for Therapy Using Our Inventive Method Using Insulin and OM, SAS, IVB, IV, and IA

Before using described inventive methods and examples, a thorough examination of the affected patients' condition needs to be evaluated. Diagnosis established according to the guide lines set by CDC rabies center of Atlanta. One need serum, cerebrospinal fluid (CSF), saliva, and biopsy of hairy skin (nape of the neck) to establish the diagnosis. For information, call CDC rabies branch to request assistance. 404-639-1050. www.cdc.gov/ncidod/dvrd/rabies/professional/Prof.forms/antem.htm

Physician contact: All the intensive care monitoring is in place. The care givers are vaccinated against rabies and protected using strict isolation of the patient and the care giver. There isn't a place to describe the extensive monitoring of the patients during the treatment. The physicians who treat rabies cases are referred to read and adopt the “Rabies Treatment Protocol (Checklist) File Format: PDF/Adobe Acrobat—Quick View Call CDC rabies branch to request assistance. 404-639-1050, 404-639-1050 or Call Dr. Willoughby at Medical College of Wisconsin to request BH4. (414) 266-2000). The physicians' who treat rabies can contact T. R. Shantha, MD, PhD, F.A.C.A; 115 Bayberry Hills, McDonough, Ga. 30253, Phone/Fax: 770-507-6564, Cell: 678-640-7705, shantha35@aol.com to the use of the present inventive method.

When treating the Rabies patients, the patient has to be cared in the ICU with strict isolation. Provisions of continued care are needed to have a team consisting of an anesthesiologist, neurologist, neurosurgeon, cardiologist, pulmonologist, critical care physician, physical therapist, and trained nursing staff. When the patient is diagnosed with rabies, please undertake the following measures.

Admit the patient to ICU; Patient is sedated (ketamine, Midazolam, barbiturates, propofol) and paralyze with non depolarizing muscle relaxants. Patients should be intubated or undergo tracheotomy with cuffed tube in order to protect the airway from aspiration secondary to copious salivation, bulbar paresis, or rabies-associated sedation.

Insert a continuous sub arachnoid intrathecal catheter at cervical or thoracic or lumbar region or insert catheter into cisterna magna (FIG. 12). Continuous epidural catheter can be inserted to SAS if the subarachnoid catheter is not available.

Need to place an Ommaya reservoir positioned in the lateral ventricle with the help of the neurosurgeon (FIG. 13).

Insert nasal olfactory mucosal delivery inventive catheter as shown and described in the FIG. 18. If it is not available, a long plastic catheter measuring 1.5 to 2 inches can be used in its place. If that is the case, run the catheter after lubrication, directed backwards abutting the anterior edge of the nose.

Central venous pressure and/or Swan Ganz catheter is used to measure the central venous pressure (CVP), temperature, and the cardiac output.

Place a nasojejunal feeding tube to maintain nutrition and fluid balance.

Otic temperature monitoring device be can be used to monitor temperature as needed. Any other temperature monitoring will do (through SGcathter, transesophageal, endotracheal, transveiscal or rectal).

Use support hose or inflatable stockings as prophylaxis against deep vein thrombosis. Physical therapy should be regularly scheduled during the period of therapeutic sedation and rabies associated paresis to avoid contractures, and DVT. Heparin 10 U/kg/hour is administered as prophylaxis. The patient should be frequently repositioned to avoid pressure ulcers and use an air floating mattress if available.

Place a Foley catheter to monitor the urinary output and have a glucose meter to measure the blood glucose levels as needed which needs to be measured 15 minutes after administration of insulin through the above described routes.

The patient needs to be monitored using EKG for any cardiac arrhythmias. Measure the cardiac output as needed.

Brain function can be monitored using Bisepctral index brain monitoring system (BIS) of the EEG. Complex signal processing algorithms are used to produce the BIS which are graded from 100 (awake) to 0 (electrical silence). The scale has been demonstrated with the hypnotic end-points of anesthesia. For the first time, we have a “window into the brain”. It allows us and anesthesiologists to dose hypnotic drugs more accurately, reduce the risk of awareness during anesthesia-sedation and/or artificially induced coma (general anesthesia) and to improve patient recovery. Complex signal processing algorithms are used to produce the BIS which is graded from 100 (awake) to 0 (electrical silence) which indicate the hypnotic end-points of sedation-anesthesia.

This is available to be used in ICU units, and the Aspect Medical Systems, Inc, Norwood, Mass. 02062 (Business Phone: 1-617-559-7000) markets it. It is a valuable piece of equipment to use in rabies patients where the care givers can look inside the rabies virus infected brain electrical activity and monitor the level of sedation needed for the patients' care. Suppress EEG recorded burst by administration of Phenobarbital, valium, versed or propofol through OM, SAS, IVB, IV, and IA routes with insulin or intravenously.

Blood should be analyzed for WBC, Platelet, HB, HMCT, ESR, CRP, electrolytes, liver function tests, another tests as needed, and correct any deficiency.

Advantages of Intranasal TRANS Olfactory Mucosal Delivery of Antirabies Therapeutic Agents with Insulin of our Invention.

Without going in to detail, we want to briefly discuss the advantage of olfactory mucosal delivery of therapeutic agents to treat Rabies. Review article by Talegaonkar and Mishra (S Talegaonkar, P R Mishra. Intranasal delivery: An approach to bypass the blood brain barrier: 200: 36: 3: 140-147) examine this subject of intranasal delivery in detail. From our research, the intranasal delivery consists of:

1. Delivering therapeutic agents through the respiratory mucosa which is absorbed through the BV of the systemic circulation;

2. Delivering of therapeutic agents through the olfactory epithelium and other nerve structures located in the roof, upper medial and lateral walls of the nose covering almost ⅓ of the nasal cavity at this region. Most of the absorbed therapeutic agents through this OM are directly delivered to CNS neuropile and the CSF in the SAS (FIGS. 7-10).

There are numerous advantages for this mode of delivery to the CNS directly. Advantages are: It is Painless, Ease of use, needed, avoids first pass metabolism which improves bioavailability over oral and rectal doses, and Nose-brain pathway allows direct delivery to the cerebral spinal fluid and to the neuropile. Compliance is not an issue which there is easy and fast delivery to any patient.

We have used ketamine, insulin, and other therapeutic agents with great success using this route. Besides the list of therapeutic agents delivered through the OM are endless. We can deliver insulin-like growth factor-I, Deferoxamine, and erythropoietin (protect the brain against stroke in animals), neuroprotective peptide to the brain to treat neuro degeneration, fibroblast growth factor-2, and epidermal growth factor which have been shown to stimulate neurogenesis in adult animals and HMAB. Our study indicates that the olfactory mucosal insulin improves memory, attention, and functioning in patients with Alzheimer's disease or cognitive impairment, senile dementia, improves memory, and mood in normal adult humans, aged, depressed, Alzheimer's, and Lyme disease. There is hardly a CNS disease that cannot be treated by using Insulin delivered through the OM with other therapeutic agents. This new method of delivery with insulin can revolutionize the treatment of Alzheimer's disease, stroke, depression, other CNS diseases and including RABIES.

General Principles of Treatment Rabies Using Insulin in Our Invention

We do not induce coma in our invention.

Our inventive treatment involves the therapy without inducing a coma. The ketamine, versed, barbiturate, propofol, and other sedatives are used to induce sedation and induce hypnotic state short of coma.

Use of Propofol to Achieve Desired Sedation and Hypnotic State with Insulin in Our Invention:

Propofol, an anesthetic, may help reduce inflammatory cytokine induced by rabies virus besides sedating and/or anesthetizing. Experimental studies show that the propofol significantly reduced the levels of LPS-enhanced TNF-alpha, IL-1 beta, and IL-6 proteins. Data from RT-PCR showed that LPS induced TNF-alpha, IL-1 beta, and IL-6 mRNA. Propofol inhibited these effects. LPS increased NO production and induced nitric oxide synthase (iNOS) expression in macrophages. Exposure of macrophages to propofol significantly inhibited the LPS-induced NO biosynthesis. The studies shows that propofol, at a therapeutic concentration, has anti-inflammatory and antioxidative effects on the biosyntheses of TNF-alpha, IL-1 beta, IL-6, and NO in LPS-activated macrophages.

The suppressive effects are exerted at the pretranslational level. (Chen R M, Chen T G, Chen T L, Lin L L, Chang C C, Chang H C, Wu C H. Ann N Y Acad Sci. Anti-inflammatory and antioxidative effects of propofol on lipopolysaccharide-activated macrophages 2005 May, 1042:262-71).

Due to above explained therapeutic advantages, its sedating-anesthetizing effects of our invention describes the use of Propofol (marketed as Diprivan) intravenously with small doses of insulin to enhance its action. The propofol is to be used to intubate the rabies patient, to insert Ommaya reservoir and to insert a Swan Ganz catheter. There are indications when the patient is anxious and is difficult. Sedation is needed which the IV propofol drip is started. Chemically, propofol is unrelated to barbiturates which it has largely replaced sodium thiopental (Pentothal) for induction of anesthesia. The recovery from propofol is more rapid and “clear” when compared with thiopental and has amnesic effects.

Propofol isn't considered an analgesic, so opioids like fentanyl may be combined with propofol to alleviate pain. It acts by potentiation of GABA_{A} receptor activity which slows the channel-closing time and acts as a sodium channel blocker. EEG research on those undergoing general anesthesia with propofol finds that it causes a prominent reduction in the brain's information integration capacity at gamma wave band frequencies. It needs trained anesthesiologist and anesthetist assistance in using these sedating therapeutic agents in rabies virus infection of the brain. For sedation use 25-100 mcg/kg/min; for Induction of unconsciousness use 1-2.5 mg/kg and the Maintenance dose is 50-200 mcg/kg/min. The drug needs to be administered, continuously; otherwise, the patient wakes up within 5-10 minutes. Use of insulin with propofol can reduce the dose and the effect may last longer at the same time reduce the cytokine load of the CNS by inhibiting the microglia and aiding the recovery of the rabies afflicted brain.

Our Invention Administers Ketamine as Anesthetic and Antirabies Therapeutic Agent with Insulin

Our inventive method uses Ketamine with insulin as sedative, antiviral, and GABA receptors antagonist. Our invention is to administer the non-competitive NMDA antagonist Ketamine directly to OM, SAS, and IVB as well as IV. The dose to be calculated based on the sedation and response. 2.2 mg doses are used in divided doses per day administered OM, SAS, IV and IVB. When used in these routes, it is used with insulin, thus, reducing the dose of the drug close to 0.5 mg per Kg. The dose as high as 48 mg per KG/per day has been used in rabies patients (Willoughby et al IBID). The anesthetic dose that I used was 2.2 mg per Kg given intravenously. The effects last many hours.

The dose given through the OM, SAS, and IVB need to be divided from the total dose and administered following the administration of insulin. The same dose can be used and can be repeated every 8-12 hours once in rabies patients. The advantage of using ketamine is the dissociative anesthetic, excellent sedative, it is an anti arrhythmic, reduces the pain perception due to its local anesthetic like effects, maintains bronchial dilatation, doesn't decrease the BP, and causes tachypnoea, with the inhibition of rabies virus multiplication and blocks the NMDA receptors.

Ketamine does cause tachycardia and elevated blood pressure initially in our use over a period of 40 years. The rabies patients have the sympathetic hyperactivity where one may have to watch for excessive discharge because it already exists due to rabies. If it is uncontrollable, you must use beta blockers given intravenously. We have used Esmolol, a short acting beta-blocker, to block the sympathetic effects of Ketamine. Other long acting beta blockers, Inderal, can be used. It is important to note and to get the desired effects of Ketamine, that it was administered in smaller doses with insulin through OM, SAS, IV and IVB routes, which reduces the sympathetic hyperactivity due to large doses of ketamine.

The effect of the rabies virus, recent, work with cats indicates that small doses of ketamine stimulate the neocortex, hippocampus, and other subcortical centers, which eventually induces the seizure activity (Kayama Y, Iwana K: The EEG, evoked potentials, and single-unit activity during ketamine anesthesia in cats. Anesthesiology 36: 316-328, 1972). This challenges the view of previous workers that ketamine induces depression of the thalamoneocortical system with activation of the limbic system, causing functional dissociation (Corssen G, Miyasaka M Domino E F: Changing concepts in pain control during surgery: dissociative anesthesia with CI-581. A progress report. Anesth & Analg 47: 746-759, 1968, Miyasaka M, Domino E F: Neuronal mechanisms of ketamine-induced anesthesia. Int J Neuropharmacol 7:557-573, 1968)

Ketamine has been known to act as a local anesthetic. According to the “gate theory of pain” of Melzack and Wall, gate theory, increased central efferent impulses can act on the gate (located in the spinal cord) and close the gate system (no feeling of pain) for all input from any site on the body (Melzack R, Wall P D: Pain mechanisms: a new theory. Science 150:971-979, 1965). Because of the central stimulating effect of ketamine, the increased efferent impulses from the center may not only close the pain perception gate, thus, causing analgesia, which blocks the hiccup centers in the cervical spinal cord and brain stem. It has been used for hiccup in the dose of 40 mg IV effectively. Its local anesthetic action of ketamine may to some extent block peripheral nerve endings that lowers the number of afferent stimuli reaching the hiccup centers (Shantha, T. R. Ketamine For the Treatment of Hiccups During and Following Anesthesia: A Preliminary Report Anesthesia And Analgesia. Current Researches VOL. 52, No. 5, September-October, 1973. Dowdy E G, Kaya K, Gocho Y: Some pharmacologic similarities of ketamine and local anesthetics. Abstracts of Scientific Papers, 1971 ASA Annual Meeting, p 165).

The report by Willoughby et al. of treating a rabies teenager successfully using ketamine and induction of coma raised hope that the treatment of others to cure this dreaded disease is at hand (Willoughby, R. E. Jr. Tieves. K. S. Hoffman. G. M. et al. (2005). Survival after treatment of rabies with induction of coma. New England Journal of Medicine 35Z. 2508-2514). Follow up studies using Ketamine to treat 4 cases of rabies similar to Willoughby et al protocol (using ketamine and inducing coma) has failed to save the patient's life (Hemachudha, T., Sunsaneewitayakul. B., Desudchit, T. et al. (2006). Failure of therapeutic coma and ketamine for therapy of human rabies. Journal of Neurovirology 12, 407-409). Latest experimental studies in primary neuron cultures and in a mouse model indicate that ketamine therapy is poor and disappointing to treat rabies.

There is no supportive evidence that excitotoxicity plays an important role in rabies virus infection (Weli, S. C., Scott, C A. Ward, C. A. and Jackson, A. C (2006). Rabies virus infection of primary neuronal cultures and adult mice: failure to demonstrate evidence of excitotoxicity. Journal of Virology 80, 10270-10273). The treatment to save lives from rabies is still elusive. The trials and experimental studies on the effect of Ketamine with the rabies virus do add to the advancement of understanding the rabies virus to some extent. This may have to be combined with other therapeutic agents as described in our invention such as use of HMAB with insulin.

Glutamate, an excitatory amino acids, have been said to participate in a role of neuronal injury in a variety of neurological diseases, including stroke, epilepsy, and various neurodegenerative disorders. There is evidence that neurotrophic viruses, including human immunodeficiency and rabies virus induces neuronal injury through N-methyl D-aspartate NMDA excitotoxicity mechanisms that the (NMDA) receptor may be one of the rabies virus receptors.

Willoughby use of ketamine for rabies was based on Tsiang et al studies which showed that the non-competitive NMDA antagonist ketamine and/or MK-801 inhibited rabies virus infection in primary neuron cultures, inhibited rabies virus genome transcription, and restricted viral spread in an experimental model of rabies in rats (Lockhart, B. P., H. Tsiang, P. E. Ceccaldi, and S. Guillemer. 1991. Ketamine-mediated inhibition of rabies virus infection in vitro and in rat brain. Antiviral Chem. Chemother. 2:9-15. Tsiang, H., P.-E. Ceccaldi, A. Ermine, B. Lockhart, and S. Guillemer. 1991. Inhibition of rabies virus infection in cultured rat cortical neurons by an N-methyl-D-aspartate noncompetitive antagonist, MK-801. Antimicrob. Agents Chemother. 35:572-574. Tsiang, H., A. Koulakoff, B. Bizzini, and Y. Berwald-Netter. 1983. Neurotropism of rabies virus; an in vitro study. J. Neuropathol. Exp. Neurol. 42:439-452). Immunofluorescence and [³⁵S] methionine labeling of infected neurons showed that 1 to 1.5 mM of ketamine inhibited viral nucleoprotein and glycoprotein syntheses and 5- to 11-fold reduction in the levels of rabies virus mRNAs, relative to those in untreated neurons. The antiviral effect was not complete.

A time-dependent recovery of viral transcription and rabies virus protein synthesis was observed without the infectious virus being released into the culture supernatant. Studies showed that the dissociative anesthetic ketamine, MK-801 and phencyclidine derivatives—noncompetitive antagonist of the N-methyl-D-aspartate (NMDA) receptor, inhibited the production (100- to 1,000-fold) of rabies virus in a dose-dependent manner. They are neuroprotective (Lockhart, B. P., H. Tsiang, P. E. Ceccaldi, and S. Guillemer IBID, Tsiang, et al 1991. 35:572-574. Tsiang, H., A. et al. 1983 J. Neuropathol. Exp. Neurol. 42:439-452. 32, 34). Attempts are being made to determine which step of the rabies virus replicative cycle in neuronal target cells is inhibited by ketamine that future possible anti-rabies virus strategies may be developed (Lockhart, B. P., H. Tsiang, P. E. Ceccaldi, and S. Guillemer. 1991, IBID.). Ketamine doesn't affect replication of herpes simplex virus, vesicular stomatitis virus, poliovirus type 1, and HIV virus.

The effects of ketamine is not a virucidal or cytopathic one, which is unlikely that ketamine had any effect on the early events of viral infection of neurons, such as binding, penetration, or uncoating. In addition, the antiviral effect wasn't related to an inhibition of cellular transcription or translation that (i) the expression of a “housekeeping” gene (p-actin) was unaffected by the drug treatment, and (ii) the inhibitory effects on viral mRNA and protein syntheses occurred to a similar extent which indicates the viral mRNA was fully translated. Studies show that peripheral treatment of rabies virus-infected rats with ketamine reduced rabies virus infection in the thalamus, cortex, and hippocampal formation in particular, the pyramidal layer of the CA I region (Lockhart, et al IBID 1991). The mechanism of ketamine on the rabies virus isn't understood clearly. Thus, the antiviral activities of ketamine and MK-801, with their neuroprotective action could represent an important potential for these drugs in the treatment of clinical rabies with other therapeutic agents as described in our invention.

Administration of Human Anti-Rabies Monoclonal Antibody (HMAB) with Insulin in Our Invention

U.S. Patent Application Publication Number: US 2009/0041777 (by Al by William D. Thomas, et al) on Human Antibodies Against Rabies And Uses; Provides a recombinant fully human anti-rabies monoclonal antibody (HMAB) that specifically binds a broad variety of rabies virus which isolates and inhibits the ability of the virus to infect cells. This is an improved method of passive immunotherapy for treating a subject infected with rabies virus in combination with other antirabies therapies described in this invention.

Our invention describes the use of these newly developed safe human MAB of the above invention through OM, SAS, IVB, IV, and IA routes and floods the CNS and PNS with, HMAB, ANA antibodies to stop the reproduction of the rabies virus and to kill the remaining rabies virus. Along with the human MAB against rabies virus, our invention involves using MAB (e.g. Etanercept) against various cytokines that are produced by the microglia and other immune system of the brain due to Innate (nonspecific) defense reaction as a first line of immune defense, responds to any infection, and recognizes characteristics common to microbial invaders. This innate reaction results in massive production of non specific cytokines affecting the CNS and its function.

Our Invention of Administration of Biopterin with Insulin During the Course of the Rabies Brain Infection and after Recovery From the Disease to Prevent Neurological Disease

Our invention uses Biopterin from day one. This use with insulin to enhance uptake by the neuropile. Biopterin is chemically similar to folic acid, one of the B type vitamins essential to cell growth of the CNS. The compound is present in limited amounts in the brain, where it is critical for manufacturing neurotransmitters such as dopamine, epinephrine, nor epinephrine, serotonin and melatonin. Biopterin controls the brain enzyme, neuronal nitric oxide synthase, maintains the tone of blood vessels which feed the brain. Oral preparations are available which should be used after intranasal administration of insulin. If injectable form is available, it should be used OM, SAS, IV, and IVB routes after appropriate dose of insulin. Paradoxically, except rabies, most of brain infections increase the level of biopterin. Willoughby et al (IBID) discovered that there is biopterin deficiency and many of the post recovery of paresis symptoms were relieved by administration of this folic acid like supplement biopterin.

Administration of Amantadine in Our Invention with Insulin

Amantadine, like ketamine, is both antiviral and blocks the NMDA receptors. The mechanism of Amantadine antiviral activity involves interference with a viral protein, M2 (an ion channel), which is required for the viral particle to become “uncoated” when taken inside a cell by endocytosis. This has been widely used against influenza B strains. It appears to be a weak NMDA receptor antagonist as well as an anticholinergic. It has antiparkinsonian effect due to increased release of dopamine and nor epinephrine from nerve endings which may result in nervousness, anxiety, agitation, insomnia, difficulty in concentrating, and exacerbations of pre-existing seizure disorders and psychiatric symptoms in patients with schizophrenia or Parkinson's disease. So, this drug needs to be used with caution or avoided these in rabies patients who already have these signs and symptoms. It can be administered if the patient is sedated and paralyzed with muscle relaxants. It is given 200-400 mg a day with insulin and if parenteral doses are available, use OM, SAS, IVB, and IV routes.

Administration of Antiviral Ribavirin Therapeutic Agent in Our Invention with Insulin

According to Dr. A. Jackson (Book on “Rabies”, page 324), the antiviral therapy with intravenous ribavirin (16 patients given doses of 16-400 mg) was unsuccessful in China. An open trial of therapy with combined intravenous and intrathecal administration of either ribavirin (one patient) or interferon-Alfa (three patients) were unsuccessful. Ribavirin is a prodrug, which when metabolized resembles purine RNA nucleotides. In this form it interferes with RNA metabolism required for viral replication. Its affects on rabies viral replication is unknown. It has effect on both DNA and RNA viruses. Ribavirin is known to enhance host T-cell-mediated immunity against viral infection through helping to switch the host T-cell phenotype from type 2 to type 1. This may explain ribavirin's antiviral activity against some viruses such as hepatitis C. The doses don't clearly interfere with replication of the virus when used without interferon. In the later stages of the rabies infection, ribavirin can be used to enhance the T cell activity in rabies patients using insulin administered through OM, SAS, IV, and IVB routes.

Blocking the Virus Spread in the CNS by Disrupting the Neurotubules Using Colchine and Vinblastine with Insulin in Our Invention

The speed of axoplasmic transport of the rabies virus is estimated to be 12 to 24 mm/day, including the time required for internalization of the virus into neurites. The virus transport can be blocked by cytochalasin, vinblastine, and colchicine, which negatively affect the production of virus in cells when the infection was established (E. Lycke and H Tsiang. Rabies virus infection of cultured rat sensory neuron. J Virol. 1987 September; 61(9): 2733-2741). They act on the neuroskeleton which includes the neurotubules of the nerve cells, their axons, and dendrites slowing down or preventing the retrograde, centripetal, inter-neuronal, centrifugal, intraneuronal and synaptic spread of the rabies virus.

Now it is understood that neurotubules play an important role in the spread of the RV. But it is still a mystery how the RV jumps billions of synaptic junctions to infect the adjoining cells and their processes. It is known that there are heavy concentrations of RV at synaptic junction, indicating there is some delay in their spread to next neuron, their process due to difficulty in jumping the synapses. It is a kind of bottle neck phenomenon.

The effect of colchicine, an inhibitor, of axonal transport due to disruption of neurotubules, on the spread of rabies virus in the central nervous system (CNS) has been investigated using Wistar rats by injecting Colchicine into the striatum at various times before and after inoculation of rabies virus into the same site. The most effective inhibitory effect was obtained by colchicine treatment applied two days before virus inoculation. Under these conditions, no fluorescent foci could be detected until day 3 post-infection, whereas, control rats exhibited infected cells within two days of post-infection. This inhibitory effect is reversible. The general consequence seems to be a delay in the rate of viral spread. However, five days after the virus challenge, some major brain areas were still partially preserved from infection (striatum, frontal cortex, pyriform cortex) indicating its therapeutic value in treating rabies and its spread. It is known that the colchicine and vinblastine breaks down the neurotubules, which inhibit or prevent the easy spread of the rabies virus from cell to cell, synapses, and neurites.

Ten days after colchicine treatment, the microtubules have recovered their capacity to transport the virus. At the onset of paralysis, the general pattern of infection in brain sections from colchicine-treated rats weren't significantly different from that of control rats. This inhibitory effect on the transport of rabies virus can be prolonged by administration of additional colchicines (Cecca 1 dl P E, Gillet J P, Tsiang. H. Inhibition of the transport of rabies virus in the central nervous system J Neuropathol Exp Neurol 1989 November; 48(6):620-30). This is of great significance in our invention, we want to use colchine and vinblastine and other neurotubules breaking therapeutic agents through OM, SAS, IVB, IV, and IA routes to prevent the propagation of the rabies virus within the CNS centrifugal spread through the neural tubules.

Colchicine is used in gout. It is phenanthrene derivative, derived from species colchicum. Each vial contains 1 mg in 2 ml of solution. Colchicine is soluble in water, alcohol and chloroform. It isn't a diuretic and doesn't influence the renal excretion of uric acid or its level in the blood. It does not alter the solubility of urate in the plasma. Colchicine is not a uricosuric agent. An acute attack of gout occurs as a result of an inflammatory reaction to crystals of monosodium urate that is deposited in the joints tissue from hyperuric body fluids, which is relieved by colchicine. Colchine as antimitotic may play a role in prevention of synthesis and maintenance of neurotubules. Interestingly enough, it has been shown that the colchicine effect is not permanent which the neurons recover and build back the neurotubules in 10 days.

The colchine is diluted to 10 to 50 microgram per ml and used OM, SAS, IV, and IVB following the administration of insulin. It can be administration by IV route in very low doses. It can be used in very small doses every other day or once a week. Local irritation of these therapeutic agents needs to be kept in mind. Hence, they are diluted to extent that they are not irritative when administered through OM, SAS, and IVB with insulin pretreatment to augment-amplify its uptake and its effect on neurotubules breakdown.

Vinblastine is a Vinca alkaloid supplied in 10 mg dose package for intravenous use (IV). It is used in Hodgkin disease, non-Hodgkin lymphoma, and mycosis fungoides, advanced testicular carcinoma, Kaposi sarcoma, choriocarcinoma, breast cancer and other cancers. Vinblastine interferes with metabolic pathways of amino acids leading from glutamic acid to the citric acid cycle and urea. Vinblastine has an effect on cell energy production required for mitosis and interferes with nucleic acid synthesis. How vinblastine acts against the neurotubules formation is not known. It is possible that it interferes with nucleic acid synthesis needed for neurotubules formation and their maintenance. We will dilute it to 10 to 50 micrograms per ml in suitable dilutant and administered OM, SAS, IV, and IVB with insulin to enhance its uptake and therapeutic effects. Due to augmentation-amplification effects of insulin, the dose of these therapeutic agents can be drastically reduced to prevent any subsequent damage to the brain.

Our inventive method of using Antirabies vaccination before PEP) and during the rabies disease. If the saliva, hair samples, and CSF are negative for rabies virus, and the patient is suspected of rabid animal bite, the PEP vaccine should be started no matter how many days has lapsed since the bite. We advise giving oral vaccine if there are early neurological symptoms without out pouring of rabies virus in the saliva or in the hair root. This method may enhance the output of ANA and clear the virus from the CNS rabies virus infection which may save the life. The vaccination using PEP method in full blown diagnosed rabies is not established that may not be needed due to centrifugal spread in the saliva, which were swallowed and stimulated in the immune system cells in the lamina propria.

Our invention relates to use of intra dermal human diploid rabies vaccine or chick embryo vaccine mixed with 2 units of insulin for faster response to the vaccination. The insulin will enhance the uptake of the rabies antigen by the Langerhans cells (FIGS. 16, 17) and augment its effect in enhancing the immune system response in the lymph nodes and other parts of the immune system. They can be administered every day till the patients recover or till we find the high ANA has developed in the blood and are in the CSF. My invention of intradermal rabies virus antigen injection with insulin can be done 2 to 4 areas of the skin—two deltoid regions and two in front of the thighs.

This method is worth trying even if the person has the full blown disease. The immunity development after intra dermal injection with insulin is different and more effective than giving subcutaneously which case the vaccine circulates all over the body to land in immune producing system. The intradermal method with insulin stimulates the cellular and Humoral component of the specific reaction at specific region compared to other methods. Our invention involves the administration of antirabies viral human diploid vaccine (HMAB) with insulin at the bite site.

Breaking of the BBB to Facilitate the Anti Rabies Therapies to be Delivered to the Neuropile with Insulin in Our Invention (FIGS. 21, 22)

There are many methods used to open up the 400 miles of BBB (FIG. 21) of the brain to allow the therapeutic agents especially HMAB to be delivered to the site of rabies virus inflammation in the neuropile (FIG. 22). The following are some of the methods available, and it is the choice of the care provider to select the method.

Hyperosmolitity Fluid administration: a high concentration of a hyperosmolar substance such as mannitol and urea in the blood can open the BBB. Osmotic shrinkage of CNS capillaries results in opening between the endothelial cells of the BBB blood vessels. This is the solid basement membrane that surrounds the sub endothelial space is cracked open (FIG. 22 arrows). This results in loosening of the end feet of the astroglia and pericytes attaches to the basement membrane. The BBB that prevented the leaking of the substance becomes leaky allowing the many therapeutic agents including the immune system components, ANA and HMAB near the vicinity of the neuropile where the rabies virus are multiplying and contributing the neuronal pathology.

The breaking of the BBB and shrinking of the endothelial lining cells is achieved by using Mannitol which is one of the commonly used in brain edema to shrink the brain during brain surgery. Diuretics added to the mannitol therapy will enhance breaking the BBB processes. I have used this method for 3 decades during neurosurgery. We have directly injected the mannitol-insulin-heparin solution into the internal carotid artery in the neck using 22 gauge catheter followed by the administration of anticancer agents to treat the brain malignant tumors. These patients responded better and lived longer with slow growth of the tumor. The same method can be use in the treatment of Rabies.

Vasoactive substances such as Bradykinin can be used to open the BBB. Insulin enhances the transfer of therapeutic agents across the BBB, and membranes covering the nervous system to neuropile. It does act as a breaker of BBB. It is physiologically broken followed by the administration of therapeutic agents in to the systemic circulation and/or OM, SAS, and IVB.

The latest studies show that IL-2 produce leak in BV and increases the delivery of the chemo therapeutic agents to the cancer site. This effect is due to permeability enhancing peptide in IL-2. The same principle can be used to break BBB by its permeability enhancing peptide (from PEP-Keck Sch. Med)) followed by the administration of antirabies therapeutic agents.

High intensity focused ultrasound (HIFU) opens BBB. It is special equipment which isn't practical in rabies cases treated in rural areas. Hypertension, Radiation, Infectious agents, Trauma, Ischemia, Inflammation, hyperbaric Pressure, Microwaves opens the BBB. We already have massive infection of the brain with the rabies viruses, hence, the BBB is broken to some extent.

Using insulin with mannitol and bradykinin with a diuretic is all that is needed to break the BBB to allow the therapeutic agents to enter the CNS to reach neuropile in enough concentrations to counter the rabies infection.

My choice is to use mannitol and insulin along with diuretics which we have used for decades in my practice which are used today. Once, the BBB is broken using any combination of above methods. Then administer therapeutic agents intravenously or through the internal carotid artery as well as through the OM, SAS, IVB, IV, and IA such as HMAH, vinblastine, colchicine, Etanercept, Progesterone with insulin.

Treatment of Vasospasm of the Rabies Infected Brain with Triple-H Therapy

The combination of induced hypertension, hypervolemia, and hemodilution known as triple-H therapy is utilized to prevent and to treat cerebral vasospasm and subarachnoid hemorrhage. We do know that the rabies is associated with vasospasm of the cerebral blood vessels. We examined the possibility of using triple H therapy to treat vasospasm in rabies brain in our invention. The triple H therapy has gained widespread acceptance over the past 20 years. The efficacy of triple-H therapy with its precise role in the management of the vasospasm in rabies afflicted brain remains uncertain due to complications that it creates in a sick patient.

Triple-H therapy carries a significant medical morbidity, including pulmonary edema, myocardial ischemia, hyponatremia, renal medullary washout, indwelling catheter-related complications, cerebral hemorrhage, and cerebral edema as well as lowering of the hemoglobin to excessively low levels. There is some emerging physiologic data suggesting that normovolemic hypertension may be the component most likely to increase cerebral blood flow in vasospasm of the afflicted brain. Due to these unexpected complications, our invention avoids triple H therapy to treat vasospasm of the brain, to treat the condition with vasodilators, and other therapeutic components of the triple H therapy when needed.

Our Invention Incorporates Intranasal Convection and Conduction Hematogenous Surface Cooling of the Rabies Afflicted Brain

Our invention of curing the rabies incorporates the cooling of the brain by using the latest and less cumbersome methods available. The Intra-Nasal Cooling System is designed to overcome limitations of other temperature reduction technologies. The limitations of using the old methods prevent the early and the rapid commencement of the patient cooling. Commercially available cooling methods induced hypothermia by invasive means such as:

1. Invasive method: which a catheter is placed in the inferior vena cava via the femoral vein, the blood is withdrawn, cooled and returned to the circulation. The invasive nature of these devices carries additional risk and complications such as bleeding, and intravascular clotting, and infection.

2. Non-invasive method: involves placing chilled water blankets in direct contact with the patient's skin.

Surface cooling devices such as water-filled blankets and pads, are easy to apply, are inefficient, cumbersome, need large refrigeration units and interfere with patient management. Intravascular cooling devices that cool the blood are more efficient than surface cooling devices. They are invasive, require large refrigeration units, and need to be placed by specially trained physicians under restricted conditions, and of course cannot be used in rabies patients due to nature of this disease. The latest method developed by Jenkins Comfort Systems (1453 Greene Street, Augusta, Ga. 30901, USA (706) 261-5404) uses Peltier Chip to cool the water jacket and keep it cool which encloses the body. We have used this simple inexpensive, effective device successfully in hyperthermia cases, where the fever was difficult to control. This device can be used with following device in addition or to cool the body to reduce the fever of the rabies patients.

The intranasal cooling (INC) device developed by Bene Chill of Santiago called the RhinoChill™ has greatly facilitated the therapeutic application of cooling the body and the organs to save the life of patients including the ones with rabies. It uses a non-invasive nasal catheter that sprays a rapidly evaporating coolant liquid into the nasal cavity and a large cavity that is a heat exchanger which lies right under the brain and the brain stem enabling safe administration of coolant therapeutic. RhinoChill™ Intra-Nasal Cooling proprietary system is portable, uses batteries and is compact for efficient use in the field which is very useful for using in rural areas for rabies cases. As a result, non-specialized medical personnel can begin the brain cooling in rabies cases and after cardiac arrest before the return of spontaneous circulation.

The RhinoChill™ Intra-Nasal Cooling System cools via two mechanisms—direct conductive heat transfer and indirect convective heat transfer. Advantage of this method in Rabies cooling is that it is not solely dependent on the presence of a circulation to cool like other methods. The rabies brain can be cooled via direct conductive heat transfer. This is very useful when the rabies is associated with massive vasospasm of the cerebral blood vessels. After the return of spontaneous circulation in the brain with vasodilatation, the presence of blood flow augments the conductive cooling with convective cooling to cool the entire brain and the body. The other advantage is that the cooling capability is sustainable throughout transportation of the patient to other facilities within or in-between hospitals.

The European clinical studies demonstrate that with the RhinoChill™ System, the brain is cooled 2-3 hours faster than by any other cooling methods started in the hospital. Rapid brain cooling during resuscitation, treatment of the rabies patients, and, other conditions will increase survival, improve neurological outcomes and potentially reduce patient time in the ICU and hospital stays. Our inventive method of cooling the brain in rabies using the RhinoChill or such devices along with other treatment using OM, SAS, IVB, IV, and IA routes of antirabies delivery of therapeutic agents can save thousands of rabies lives form certain deaths with intact neurological function and with the least disability. It is important to bear in mind that chilling through the nose can result in nasal mucosal damage with ulceration due to dryness of the coolant which can damage the sensitive olfactory receptor cells resulting in partial and complete loss of olfactory sensation (loss of smell).

The unexpected sudden cardiac arrest (SCA) is a leading cause of death in the United States, Canada, Europe, and other parts of the world. The annual incidence of SCA in North America is almost 300, 0000 and about 700,000 patients in Europe. More than 50% of patients die before leaving the hospital, and the majority of deaths are attributed to post resuscitation myocardial dysfunction. Furthermore, approximately 30% of survivors exhibit permanent brain damage. Systemic hypothermia initiated after resuscitation, during coma, during cardiac arrest has been shown to improve survival and better long-term neurologic outcome, improved successes in defibrillation after cardiac arrest (The Hypothermia after Cardiac Arrest Study Group: Mild therapeutic hypothermia to improve the neurologic outcome after cardiac arrest. N Engl J Med 2002; 346:549-556. Bernard S A, Gray T W, Buist M D et al: Treatment of comatose survivors of out-of hospital cardiac arrest with induced hypothermia. N Engl J Med 2002; 346:557-563. Boddicker K A, Zhang Y, Zimmerman M B, Davies L R, Kerber R E. Hypothermia improves defibrillation success and resuscitation outcomes from ventricular fibrillation. Circulation 2005; 111:3195-201. Abella B S, Zhao D, Alvarado, Hamann K, Vanden Hoek T L, Becker L B. Intra arrest cooling improves outcomes in a murine cardiac arrest model. Circulation. 2004; 109:2786-91)

Nasopharyngeal cooling (NPC) is a feasible method for inducing hypothermia in or out-of-hospital settings in rabies patients as well as in cardiac arrest, stroke, and head injury patients. Studies have shown that the NPC initiated during CPR significantly improves survival and neurologic outcome in porcine models of cardiac arrest (Tsai M S, Barbut D, Tang W, et al: Rapid head cooling initiated coincident with cardiopulmonary resuscitation improves success of defibrillation and post-resuscitation myocardial function in a porcine model of prolonged cardiac arrest. J Am Coll Cardiol 2008; 51. 1988-1990. Tao Yu, Denise Barbut, Giuseppe Ristagno, et al. Survival and neurological outcomes after nasopharyngeal cooling or peripheral vein cold saline infusion initiated during cardiopulmonary resuscitation in a porcine model of prolonged cardiac arrest. Crit Care Med 2010 Vol. 38, No. 3, 916-921).

Surface cooling methods can't be utilized during rabies because they are cumbersome and cooling rates are too slow which will have very little cooling effect on the brain within the relatively short resuscitation period in rabies patients. Intra-nasal cooling (INC) initiated at the start of rabies cases which I have named BRAIN CARDIO PULMONARY RESUSCITATION (BCPR), significantly improves the success of recovery of rabies patients with brain protection from the rabies damage. The BCPR term should be used for all CPR instead of plain CPR which gives importance to the brain that is the organ we are trying to prevent the damage by restoring the function of the heart and lungs during resuscitation.

I believe that the INC cooling results in more significant cooling in the deeper regions of the brain which are the inferior aspect of the frontal lobe, hypothalamus-pituitary regions, and the brain stem, emerging cranial nerves from the brain stem. All are closer to the source of INC method. INC may provide enough cooling in the regions of the brain responsible for the beneficial effect during no-flow and low-flow states in rabies afflicted brain. Intra-nasal cooling system is an improvement both in speed of cooling and ease of administration, when feasible must be adopted on rabies patients with our inventive methods.

Our Inventive Method of Subjecting the Rabies Afflicted Brain Under Hydrogen Sulfide Induced Hibernation (Suspended Animation)

Hibernation or suspended animation is a dormant state resembling sleep over the winter while living off reserves of body fat, with a decrease in body temperature, pulse rate and sluggish metabolism. Animals hibernate which includes the bears, bats, and many amphibians. Nature is plentiful in organisms that can and do reversibly arrest their essential life processes for weeks, months, and in some cases for several years at a time. Scientists describe these phenomena by a variety of terms—quiescence, torpor, hibernation, among others—but all represent different degrees of suspended animation, a dramatic reduction of both energy production (metabolism) and energy consumption (cellular activity).

Recently, the use of Hydrogen sulfide to induce artificial hibernation in the laboratory has created stir in the scientific community. The interesting observation is more organisms in this state enjoy unusual resistance to environmental stresses, such as temperature extremes, oxygen deprivation and even physical injury. If we can produce suspended animation for two weeks in rabies affected brain, we can save 55,000 lives a year. Hydrogen sulfide is the chemical compound with the formula H₂S. It is a colorless, poisonous, flammable gas with the characteristic of the foul odor of rotten eggs. The human body produces small amounts of H₂S which it uses as a signaling molecule. A signaling molecule is a chemical involved in transmitting information between cells. It is a heavier than air; a mixture of H2S and air is explosive. A solution of hydrogen sulfide in water is my choice to use in rabies till the development of a safe system using it in gaseous form.

Hydrogen sulfide is a poison exerting its adverse effect on different systems in the body which the nervous system is most affected. The toxicity of H₂S is due to formation of a complex bond with iron in the mitochondrial cytochrome enzymes which blocks oxygen from binding and stopping cellular respiration. At some threshold level, around 300-350 ppm, the oxidative enzymes become overwhelmed and stops functioning. Treatment of H2S poisoning involves immediate inhalation of amyl nitrite, injections of sodium nitrite, inhalation of pure oxygen, administration of bronchodilators to overcome eventual bronchospasms, and in some cases hyperbaric oxygen therapy (HBO). The HBO therapy remains controversial.

Less than 10 ppm has an exposure limit of 8 hours per day. 10-20 ppm is the borderline concentration for eye irritation. 50-100 ppm leads to eye damage, and at 100-150 ppm the olfactory nerve is paralyzed after a few inhalations, the sense of smell disappears, together and the awareness of danger. 320-530 ppm leads to pulmonary edema with the possibility of death. 530-1000 ppm causes strong stimulation of the central nervous system and rapid breathing, leading to loss of breathing. 800 ppm is the lethal concentration for 50% of humans for 5 minutes exposure (LC50). Concentrations over 1000 ppm cause immediate collapse with loss of breathing, even after inhalation of a single breath.

In 2005, it was shown that mice can be put into a state of suspended animation-like hypothermia by applying a low dosage of hydrogen sulfide (81 ppm H₂S) in the air. The breathing rate of the animals sank from 120 to 10 breaths per minute and their temperature fell from 37° C. to just 2° C. above ambient temperature (in effect, they had become cold-blooded). The mice survived this procedure for 6 hours and afterwards showed no negative health consequences. The blood pressure of mice treated in this fashion with hydrogen sulfide did not significantly decrease. If the H₂S-induced hibernation can be made to work in humans; this could be useful in the emergency management of severely injured patients, in the conservation of donated organs, comatose patients with head injury, stroke, and in rabies. Hypothermia induced by hydrogen sulfide for 48 hours was shown to reduce the extent of brain damage caused by experimental stroke in rats (Mark B. Roth and Todd Nystul. Buying Time in Suspended Animation. Scientific American, 1 Jun. 2005).

As mentioned above, the hydrogen sulfide binds to cytochrome oxidase which prevents the oxygen from binding, leading to the dramatic slowdown of metabolism. Animals and humans naturally produce some hydrogen sulfide in their bodies, which researchers have proposed, that the gas is used to regulate metabolic activity and the body temperature. This would explain the above findings (Roth IBID). In February 2010 TED conference, Mark Roth announced that hydrogen sulfide induced hypothermia had completed Phase I clinical trials. He estimated that further trials would take ‘a few years (http://www.ted.com/talks/lang/eng/mark_roth_suspended_animation.html).

How we Use the H2S in the Treatment of Rabies in Our Inventive Method

H₂S is highly toxic and an explosive in the form of a gas when mixed with air and/or oxygen. We can mix only 0.0005 parts per million and use with ventilators. All the electrical and static electricity should be avoided to prevent explosion. The gaseous state of H2S isn't practical due to its explosive nature. We propose using the water dissolved H₂S delivered OM, SAS, IA, and IVB as liquid to only hibernate or put the brain in a suspended animation for the prescribed amount of time. We propose to use enough H₂S liquid to reduce the brain temperature between 30 and 34 degree Fahrenheit. It should be used judiciously with continuous monitoring of vital functions. The dose can be repeated as desired. We are using this method only on the brain. We will be able to obtain desired protection by inducing hypothermia which reduces the cerebral metabolism and preserves the neurons from the onslaught of rabies virus.

WE CALL IT BRAIN HIBERNATION OR BRAIN SUSPENDED ANIMATION as opposed to whole body hibernation. The reduced brain temperature can reduce the pathogenicity and the spread of RV within the CNS which allows the immune system to respond. Once proper ANA levels are reached in the blood and CSF, the hibernation method is slowly terminated and allows the ANA to attack the virus in the brain and clear the virus. The rest of the body continues to function like normal. In such a state, we may have to maintain the vital functions like a decerebrate person. This form of hibernation method can be used in cardiac arrest, stoke, other types of brain injury, and treatment of brain tumors. H2S induced hibernation has immense value in the transporting the transplant organs for long distances and to preserve the organs for a long time until a suitable recipient is found. If this becomes feasible, it can have great impact in organ transplantation and saving the brain due to any destructive CNS pathology.

During this period of hibernation, it is important to continue vital function support of fluids, urine output, CV function, BP, respiration, nutrition, and proper acid base balance. We will administer Biopterin (BH4) as advocated by Dr. Willoughby protocol: BH4 (non-CNS) doses: 80 mg every 8 hours (pediatric 2 mg/kg/Q8h); Coenzyme Q10 1200-2400 mg daily (pediatric 30-60 mg/kg/day) and Consider zinc sulfate 50 mg PO every 8 hours (pediatric 1 mg/kg Q8h). According to Willoughby, BH4 deficiency should flatten the EEG and reduce cranial artery flow mimicking cerebral death (a kind of stoke). This is due to loss of EEG in rabies. It has been correlated with middle cerebral artery spasm by transcranial Doppler. EEG has returned with improvement in blood velocity by using Biopterin. Standard criteria for brain death don't apply in this deficiency. Diagnosis of brain death requires anatomic (biopsy or neuroimaging) evidence for irreversible brain damage or a brain flow scan showing zero flow intracranially. The evaluation of rabies patients with flat EEG isn't good enough to establish the diagnosis of brain death to stop providing anti rabies and resuscitation therapies.

Other Neuroprotective Therapeutic Agents Used Against The Rabies with Insulin in Our Invention

Microglial Inhibitors such as Estriol and progesterone, Minocycline, Ketamine, propofol, Vitamin A (Retinoic acid inhibits expression of TNF-alpha and iNOS in activated rat microglia), vitamin D3 (anti-inflammatory effect on microglia), and vitamin E; Hyperbaric therapy, Cortisol, Resveratol (inhibits nitric oxide and TNF-alpha production by lipopolysaccharide-activated microglia), lowers the brain temperature to inhibit the inflammatory cytokines (TNF-alpha, interleukin (IL)-12, IL-6, and nitric oxide-NO) production by activated microglia cell as well as Celebrex, aspirin and/or indomethacin for inhibition of nitric oxide synthase induction using any described suitable routes with insulin.

Estrogens and progesterone are well known to exert anti-inflammatory effects outside the central nervous system (CNS). They have been shown to exert neuroprotective effects in the CNS after several types of injury which includes the neurodegeneration, post traumatic stress disorder (PTSD). Estriol, and progesterone. The concentrations consistent with late pregnancy, inhibits NO and TNF-alpha production by activated microglia. This suggests that hormone inhibition of microglial cell activation may contribute to the decreased severity of multiple sclerosis symptoms commonly associated with pregnancy. Administration of these hormones with insulin of our insulin to Rabies patients can prevent the neurological damage by inhibiting the rabies virus activated cytokine production by microglia.

The use of cortisol is controversial in rabies. The current studies demonstrate that cortisol inhibits release of TNF-alpha from LPS-treated microglial cells. Collectively, the data suggests that, although, cortisol may be directly toxic to neurons, which the hormone may indirectly protect neurons by blocking the production of cytotoxic molecules by microglia (Drew P D, Chavis J A. Brain Res Bull. 2000 Jul. 15; 52(5):391-6).

Our inventive method includes Vitamin E with insulin is one of the supportive nutritional supplement. Vitamin E, in addition to the beneficial effects of providing direct antioxidant protection to neurons as reported by others. Vitamin E may provide neuroprotection in vivo through suppression of signaling events necessary for microglial activation.

Our inventive method of curing the rabies includes the use of curcumin with insulin for the treatment of both NO and microglial cell-mediated neurodegenerative disorders which rabies induced cytokine effect. The curcumin is a powerful inexpensive antioxidant. It suppresses both mRNA and protein levels of inducible nitric oxide synthase (iNOS), indicating that this natures preparation may affect iNOS gene expression process. The studies showed that the curcumin altered biochemical patterns induced by LPS such as phosphorylation of all mitogen-activated protein kinases (MAPKs), and DNA binding activities of nuclear factor-kappaB (NF-kappaB) and activator protein (AP)-1, which is assessed by reporter gene assay. By analysis of inhibitory features of specific MAPK inhibitors, a series of signaling cascades including c-Jun N-terminal kinase (JNK), p38 and NF-kappaB was found to play a critical role in curcumin-mediated NO inhibition in microglial cells (Jung K K, Lee H S, Cho J Y, Shin W C, Rhee M H, Kim T G, Kang J H, Kim S H, Hong S, Kang S Y. Inhibitory effect of curcumin on nitric oxide production from lipopolysaccharide-activated primary microglia Life Sci. 2006 Oct. 19; 79(21):2022-31).

We incorporate the use of minocycline to suppress the cytokine production by microglia during rabies in our invention. Minocycline, a second-generation tetracycline, has profound anti-inflammatory properties in the CNS mediated by inhibition of microglia (Nikodemova M, Duncan I D, Watters J J. J Neurochem. 2006 January, 96(2):314-23). Our inventive method uses the minocycline with insulin as part of the therapy. Hypoxia is one of the important physiological stimuli that are often associated with a variety of pathological states such as ischemia, respiratory diseases, and tumor genesis. In the central nervous system, hypoxia is accompanied by cerebral ischemia that causes neuronal cell injury and induces pathological microglial activation, which it happens in rabies.

It has been shown that the hypoxia induces inflammatory activation of cultured microglia, and the hypoxic induction of nitric oxide production in microglia is mediated through p38 mitogen-activated protein kinase pathway. Experimental data show that the minocycline, a tetracycline derivative, suppresses the hypoxic activation of cultured microglia by inhibiting p38 mitogen-activated protein kinase pathway. The drug markedly inhibited hypoxia-induced production of inflammatory mediators such as nitric oxide, TNF alpha, and IL-1 beta as well as iNOS protein expression. The signal transduction pathway that leads to the activation of p38 mitogen-activated protein kinase was the molecular target of minocycline. Thus, the known neuroprotective effects of minocycline in animal models of cerebral ischemia may be partly due to its direct actions on brain microglia. It is important to include the minocycline at least in the early stages of the rabies disease administered with insulin. (Suk K. Minocycline suppresses hypoxic activation of rodent microglia in culture. Neurosci Lett. 2004 Aug. 12; 366(2):167-71). If it is feasible, subjection of the Rabies patients to hyperbaric therapy (HBO) which may protect the brain, heart, lungs, and save the life. This form of therapy has never been advocated or tried before to treat this dreaded disease. If the facilities are found, we want to incorporate HBO as par rabies therapy.

Supportive Therapies Used with Our Invention

All rabies patients should be considered for the following supportive therapies: Consider fosphenytoin and muscle relaxants for seizures. High dose vitamin B1, IV, and oral, vitamin B complex, and Vitamin C1 to 5 grams IV, Vitamin A, and Calcium channel blocker Nimodipine-Improvement of neurologic deficits caused by vasospasm, IV Nitroglycerin for vasodilatation if needed. Cardiac supportive therapies: Zinc, Vitamin D3, and Melatonin are all considered. UbiquinolCoQ10 is a must to protect the heart. Naltrexone may be needed to wake the patient when the sedation is terminated and muscle relaxants are reversed. Consider a full “mitochondrial cocktail” preventively as described in Willoughby protocol:

1. L-carnitine 330 mg PO daily (pediatric 20 mg/kg every 8 h)

2. Coenzyme Q10—see above

3. B complex pills: 1 pill PO BID

4. Creatine (usually at health food store) 3 g daily

5. Mg Orotate (health food store) 200 mg daily

6. Vitamin E (tocopherol) 200 U daily

7. Vitamin C—1000 to 2000 mg iv or orally

8. Add alpha-lipoic acid 80 mg PO daily (pediatric: 2 mg/kg daily)

### EXAMPLES OF OUR INVENTIVE METHOD FOR TREATING RABIES PATIENTS ARE OUTLINED BELOW

### Examples 1

Patients are prepared as describe above with all the monitoring in place.

Then administer insulin through the OM, SAS, and IVB in the doses of 5, or 10 or 15 units at each administration route and monitor for blood sugar.

After 15 minutes, administer ketamine for sedation and as antiviral therapeutic agents against rabies virus.

Administer midazolam to augment the sedation

### Examples 2

Patients are prepared as describe above with all the monitoring in place.

Then administer insulin through the OM, SAS, IV and IVB in the doses of 5-10 units at each administration route and monitor for blood sugar.

After 15 minutes, administer ketamine for sedation and as antiviral therapeutic agents against rabies virus. Administer midazolam to augment the sedation.

Then administer the Human antirabies monoclonal antibodies (HMAB) through the olfactory mucosa, intrathecal into the verticals through OM, SAS, and IVB catheter system and IV or intra arterial (IA) through the carotid arteries.

### Examples 4

Patients are prepared as describe above with all the monitoring in place.

Use propofol to induce sedation with insulin

Then administer insulin through the OM, SAS, and IVB in the doses of 5-15 units at each administration route and monitor for blood sugar.

After 15 minutes, administer ketamine for sedation and as antiviral therapeutic agents against rabies virus. Administer midazolam to augment the sedation

Then administer the Etanercept though OM, SAS, and IVB routes to reduce the cytokine induced by rabies virus induced viral encephalopathy.

Follow this with administration of Human antirabies monoclonal antibodies (HMAB) through the olfactory mucosa, intrathecal into the verticals through OM, SAS, IV, and IVB catheter system

### Examples 5

Patients are prepared as describe above with all the monitoring in place.

Use propofol and/or ketamine with midazolam to induce sedation with insulin

Then administer insulin through the OM, SAS, IV, and IVB in the doses of 5-15 units or more at each administration route and monitor the blood sugar

After 15 minutes, administer ketamine for sedation and as antiviral therapeutic agents against rabies virus. Administer midazolam to augment the sedation

Then administer the Colchicine IV and/or OM, SAS, and IVB routes to reduce the cytokine induced by rabies virus induced viral encephalopathy and to disorganize the neuroskeleton net work including neurotubules to prevent the RV spread within the CNS.

Follow this with administration of Human antirabies monoclonal antibodies (HMAB) through the olfactory mucosa, intrathecal and into the verticals through OM, SAS, and IVB catheter system and intravenous and/or intra-arterial (IVA) routes.

### Examples 6

Patients are prepared as describe above with all the monitoring in place.

Use propofol and/or ketamine with midazolam to induce sedation with insulin

Then administer insulin through the OM, SAS, and IVB in the doses of 5-15 units at each administration route and monitor for blood sugar

After 15 minutes, administer ketamine for sedation and as antiviral therapeutic agents against rabies virus

Then administer the vinblastine IV and/or OM, SAS, IA, and IVB routes to reduce and to disrupt the neurotubules.

Follow this with administration of Human antirabies monoclonal antibodies (HMAB) through the olfactory mucosa, intrathecal into the verticals through OM, SAS, IVA, and IVB catheter system

### Examples 8

Patients are prepared as describe above with all the monitoring in place.

Use propofol and/or ketamine with midazolam to induce sedation with insulin

Then administer insulin through the OM, SAS, and IVB in the doses of 5-15 units at each administration route and monitor for blood sugar

Then administer the Etanercept IV and/or OM, SAS, and IVB routes to reduce the cytokine induced by rabies virus induced viral encephalopathy.

Follow this with administration of Human antirabies monoclonal antibodies (HMAB) through the olfactory mucosa, intrathecal into the verticals through OM, SAS, and IVB catheter system

Administer biopterin to preserve the brain deficiency of this folic acid like deficiency with insulin

### Examples 9

### Induction of Hypothermia Using RhinoChill Device in Rabies Patients is Part of Our Invention

Patients are prepared as describe above with all the monitoring in place.

Use propofol and/or ketamine with midazolam to induce sedation with insulin

Our present inventive method to cure rabies incorporates the method of inducing hypothermia of the brain. The rabies patients are placed in a supine position on air floated mattress if possible. The core temperature of the patients is obtained using Auricular temperature probe, esophageal, Foley catheter, and Swan Ganz catheter temperature probes, or though a temperature probe introduced along with or incorporated into the Ommaya reservoir pump. The head cooling is induced by using the Rhinochill (Benechill Inc, San Diego, Calif.) nasal catheter system of the device. The Rhinochill device sprays a liquid perfluorochemical into the nasal cavity.

The liquid is volatile and evaporates instantaneously, which removes heat from the nasal cavity and nasopharyngeal cavity. The cold is transmitted to the brain predominantly to the blood in the blood vessels of the carotid arterial and cranial-cerebral-pharyngeal-nasal venous system (hematogenous), through the sub mucosal nasal and pharyngeal blood vessel plexuses. The coolant temperature is transmitted to the hemispheres and brain stem through the wall of the nasal and oral pharyngeal walls by direct convection.

The intranasal catheters were positioned in the nostrils, and perfluorochemical was delivered at 1 mL/kg/min mixed with oxygen. Once, core temperature (The core temperature of the patients is obtained using Auricular temperature probe, esophageal, Foley catheter and Swan Ganz catheter temperature probes, or though a temperature probe introduced along with or incorporated into the Ommaya reservoir pump) reaches 33-34-35° C., or less, if all the vital signs are stable the cooling is continued. If the vital signs deteriorate, specially, the function of the heart and the lungs which is related to induction of hypothermia. The cooling is stopped and restarted based on the vital signs recovery and progress of the rabies treatment.

Once the cooling is stopped after reaching the desired levels, don't attempt to warm the body by artificial methods unless the life threatening situation is presented due to hypothermia. Let the body recover to the desired temperature levels by natural means. This method can be used continuously, on hourly basis or intermittently which the following method can be adopted. Start the INC and administer till the desired temperature is reached. Once the desired temperature is reached, maintains the temperature by turning INC Off and ON as the patient care needs which the desired temperature are achieved without jeopardizing the patent safety.

During cooling, the present inventive method of OM, SAS, IVA, and IVB delivery of antirabies therapeutic agents are continued as described in the above examples to facilitate the clearing of the virus from the CNS and to restore the normal physiological state to the CNS, and their effect on the rest of the body.

This form of hypothermia is continued for days. With the lapse of time, the immune system recovers and starts producing antirabies neutralizing antibodies (ANA) which will clear the remaining virus from the brain.

Heating and cooling of the brain can break the BBB; make it leakier which allows the RV antibodies inside the brain. Measurement of the titers of antirabies antibodies in the CSF gives us the clue that the immune system is responding, Its effects on the CNS attacking the remaining viruses and possibility saving the life of the patient.

### Examples 10

This inventive method involves Induction of hibernation-suspended animation state by using injection of hydrogen sulfide dissolved in water.

1. Patients are prepared as describe above with all the monitoring in place.

Use propofol and/or ketamine with midazolam to induce sedation with insulin

Our present inventive method to cure rabies incorporates the method of inducing hypothermia-induced hibernation using hydrogen sulfide in water soluble form. The rabies patients are placed in a supine position on air floated mattress if possible. The core temperature of the patients is obtained using Auricular temperature probe or though a temperature probe introduced along with or incorporated into the Ommaya reservoir pump measures the core temperature of the brain.

1. The H2S is introduced to the brain through OM, SAS, IVB, and carotid arterial system.

2. Monitor the temperature and continue cardiac, pulmonary, renal and nutritional supportive therapies till the patient recovers

3. Administer HMAB also as described above.

### Examples 11

This inventive method involves Breaking the BBB and administering HMAB and other antirabies therapeutic agents through OM, SAS, and IVB, IV, and IA routes

Patients are prepared as describe above with all the monitoring in place.

Use propofol and/or ketamine with midazolam to induce sedation with insulin.

There are many methods used to open BBB of the brain to allow the therapeutic agents, especially HMAB, to be delivered to the site of rabies virus infection in the CNS. We use the use high concentration of a hyperosmolar mannitol intravenously with a diuretic. By the end of the administration, the Osmotic shrinkage of CNS capillaries results in opening between the endothelial cells of the BBB blood vessels and opens the solid basement membrane that surrounds the sub endothelial space is broken open (FIGS. 21, 22). The BBB becomes leaky allowing many therapeutic agents including the immune system components, ANA and HMAB close to the vicinity of the neuropile where the rabies viruses are multiplying.

Administer the HMAB intravenously so that they can be transferred to the neuropile though the leaky BBB to clear the virus.

Using insulin with mannitol and bradykinin with diuretic can also be used, and they make the BBB even more leaky to allow the anti rabies virus therapeutic agents to reach the neuropile to clear the virus, maintain the integrity of CNS and its function.

Numerous modifications and alternative arrangements of steps explained herein may be devised by those skilled in the art without departing from the spirit and the scope of the present invention with the appended claims are intended to cover such modifications and arrangements. The present invention is described above with particularity and detail in connection with that deemed to be the most practical and preferred embodiments of the invention.

It will be apparent to those of ordinary skill in the art that numerous modifications, including, but not limited to, variations in size, materials, shape, form function and manner of procedure, assembly and use may be made. While the preferred embodiment of the present invention has been described, it should be understood that various changes, adaptations, and modifications may be made thereto. It should be understood, that the invention is not limited to details of the illustrated invention.

There are prophylactic vaccines available to prevent the development of rabies after the bite from a rabid rabies infected animal since 1885. If pre and post exposure, prophylaxis is ignored, full blown rabies develops. Once the illness develops, there is no cure and the 100% preventable disease becomes 100% fatal universally.

While the preferred embodiment of the present invention has been described, it should be understood that various changes, adaptations, and modifications may be made thereto. It should be understood, therefore, that the invention is not limited to details of the illustrated invention examples.

What is claimed is:

1. A method of treating rabies, comprising the step of administering a therapeutically effective amount of insulin through selected special routes.

2. The method of treating rabies according to claim 1, further comprising the step of administering antirabies therapeutic agents to the afflicted brain through olfactory mucosa (OM), intrathecally (SAS), intravenous (IV) and intra-arterial (IA), and interventricular (IVB) routes.

3. The method of treating rabies according to claim 2 further comprising the step of using a selected combination of antirabies therapeutic, pharmaceutical, biochemical, and biological agents or compounds delivered to the olfactory area of the nose to deliver these agents directly to the brain and cerebrospinal fluid.

4. The method of treating rabies according to claim 2, further, comprising the step of administering antirabies therapeutic, pharmaceutical, biochemical, and biological agents or compounds with insulin through an intrathecal catheter to the cerebrospinal fluid in the subarachnoid space and cisterna magna.

5. A method of treating rabies according to claim 2, further comprising the step of administering antirabies therapeutic, pharmaceutical, biochemical, and biological agents or compounds through the Ommaya reservoir into the ventricles of the brain.

6. A method of treating rabies according to claim 2, further comprising the step of administering antirabies therapeutic agents, human monoclonal antibodies (HMAB), anti TNF monoclonal antibodies Etanercept, with insulin through the olfactory mucosa to the brain, intrathecal through the subarachnoid space catheter and into the ventricles of the brain through the Ommaya reservoir.

7. A method of treating rabies according to claim 2, further, comprising the step of administering antirabies therapeutic agent ketamine, MK-801, and caspase with insulin through the olfactory mucosa to the brain, intrathecal through the subarachnoid space catheter and into the ventricles of the brain through the Ommaya reservoir.

8. A method of treating rabies according to claim 2, further, comprising the step of administering antirabies and neuroprotective therapeutic agent Epoetin Alfa (Epogen, Procrit), insulin like growth factor-1, fibroblast growth factor-2, epidermal growth factor, platelet derived growth factor, estrogen, progesterone with insulin through the olfactory mucosa to the brain, intrathecal through the subarachnoid space catheter and into the ventricles of the brain through the Ommaya reservoir.

9. A method of treating rabies according to claim 2, further comprising the step of administering antirabies therapeutic agent colchicine and vinblastine, with insulin through intravenously, intra arterially, through the olfactory mucosa to the brain, intrathecal through the subarachnoid space catheter into the ventricles of the brain through the Ommaya reservoir.

10. A method of treating rabies according to claim 2, further comprising the step of administering antirabies therapeutic agent after breaking the blood brain barrier, administering human monoclonal antibodies, Etanercept, biological neuroprotective nerve growth factors, ketamine with insulin intravenously, intra arterially, through the olfactory mucosa to the brain, intrathecal through the subarachnoid space catheter into the ventricles of the brain through the Ommaya reservoir.

11. A method of treating rabies according to claim 2, further comprising the step of inducing suspended animation of the brain by administering a therapeutic amount of hydrogen sulfide by administering liquid hydrogen sulfide intra carotid arteries, through the olfactory mucosa to the brain, intrathecal through the subarachnoid space catheter into the ventricles of the brain through the Ommaya reservoir to preserve the brain from further damage by the rabies virus.

12. A method of treating rabies according to claim 2, further comprising the cooling of the brain base, brain stem, undersurface of the optical surface of the frontal lobe, hypothalamus, thalamus, pituitary gland through the intranasal nasal canula by infusing surface coolant using Rhinochill device to preserve the brain from further damage by the rabies virus.

13. A method of treating rabies animal bite post and pre exposure prophylaxis (PEP), comprising the step of administering a therapeutically effective amount of human diploid antirabies vaccine (HDCV) or purified chicken embryo cell vaccine (PCECV); mixed with 2 units of insulin intradermal to enhance and stimulate the production of antirabies neutralizing antibodies rapidly.

14. A method of treating rabies according to claim 2, further comprising the step of administering antiviral therapeutic agents, ribavirin, amantatidine, interferon's, and antimalarial.

15. A method of treating rabies according to claim 2, further comprising the step of administering a supportive therapies by administering at least one agent selected from the group consisting of biopterin, high dose Vitamin B1, Vitamin B complex, Vitamin C, Vitamin A, vitamin E, Calcium channel blocker Nimodipine, IV Nitroglycerin, Cardiac supportive therapies, Zinc, Vitamin D3, Melatonin, UbiquinolCoQ10, L-carnitine, Creatine, Mg Orotate, alpha-lipoic acid, curcumin, estrogens, progesterone, minocycline, and biopterin.

16. A method of treating rabies according to claim 2, further comprising the step of administering at least one agent selected from the group consisting of propofol, ketamine, midazolam, anticonvulsant, barbiturates, hypnotics, analgesics, antipsychotic agents for sedation, hypnotic effects, to reduce brain metabolism, and anesthesia.
